(19) United States
(12) Patent Application Publication
(10) Pub. No.: US 2022/0262458 A1
(43) Pub. Date: Aug. 18, 2022

(54) DETECTING NEURALLY PROGRAMMED TUMORS USING EXPRESSION DATA

(71) Applicant: Genentech, Inc., South San Francisco, CA (US)

(72) Inventors: Yasin SENBABAOGLU, South San Francisco, CA (US); Christine Carine MOUSSION, South San Francisco, CA (US)

(21) Appl. No.: 17/629,327

(22) PCT Filed: Jul. 24, 2020
(86) PCT No.: PCT/US2020/043363

§ 371 Date: Jan. 21, 2022

Related U.S. Application Data
(60) Provisional application No. 62/949,025, filed on Dec. 17, 2019.
(60) Provisional application No. 62/878,095, filed on Jul. 24, 2019.

(51) Int. Cl. G16B 20/00; G16B 40/30

(52) U.S. Cl. CPC G16B 20/00; G16B 40/30

(57) ABSTRACT

Embodiments disclosed herein generally relate to classifying a tumor, based on gene expression data, as being neurally related or non-neurally related. The tumor may be classified using a machine-learning model, which may have been trained to differentiate gene-expression data associated with neuronal or neuroendocrine tumors from gene-expression data associated with non-neuronal and non-neuroendocrine tumors. Differential treatment and/or treatment recommendations may be provided based on the classification. First-line checkpoint blockade therapy may be used or recommended when a tumor is identified as being non-neurally related, and a combination therapy (e.g., initial chemotherapy and subsequent checkpoint blockade therapy) may be used or recommended when a tumor is identified as being neurally related.

### CROSS-REFERENCE TO RELATED APPLICATIONS

This application claims the benefit of and the priority to U.S. Provisional Application No. 62/878,095, filed on Jul. 24, 2019 and U.S. Provisional Application No. 62/949,025, filed on Dec. 17, 2019. Each of these applications is hereby incorporated by reference in its entirety for all purposes.

### FIELD

Methods and systems disclosed herein relate generally to detecting whether tumor data corresponds to a neurally programmed tumor. Specifically, a classifier can process gene expression data to detect whether a tumor is a neurally programmed tumor.

### BACKGROUND

Cancer is a heterogeneous disease and even individuals that present with the same type of tumor may experience very different disease courses and show different responses to therapies. The identification of groups of subjects that show different prognosis (patient stratification) represents a promising approach for the treatment of cancer. For example, multiple treatment options are available to treat a subject having tumors. One treatment option includes immune checkpoint blockade therapy. Immune checkpoints promote T-cell activation. Immune checkpoint blockade therapy aims to inhibit immune suppressor molecules and that otherwise suppress T-cell activity. In some instances, this can promote self-reactive cytotoxic T cell lymphocyte activity against tumors. However, immune checkpoint blockade therapy—like many treatment options—is not effective at treating all tumors. As another example, the efficacy of chemotherapy may differ dramatically across disease stages, cancer types, subject groups, and other known or unknown predictive characteristics. Thus, it would be advantageous to be able to better characterize individual tumors, so as to determine whether each of treatment options (e.g., immune checkpoint blockade therapy) is likely to be effective in treating a subject with the tumors or whether a personalized combination of treatments would be better suited for each of the subclasses of tumors identified.

### SUMMARY

In some embodiments, a computer-implemented method is provided for identifying a gene-panel specification. A set of training gene-expression data that corresponds to one or more subjects is accessed. Each training gene-expression data element of the set of training gene-expression data elements having been generated based on a sample collected from a corresponding subject of the one or more subjects having a tumor. Each training gene-expression data element of the set of training gene-expression data elements can indicate, for each gene of a set of genes, an expression metric corresponding to the gene. Each of the set of training gene-expression data elements is assigned to a tumor-type class. The assignment includes assigning each of a first subset of the set of training gene-expression data elements to a first tumor-type class. The first subset includes a training gene-expression data element for which the tumor was a neuronal tumor. The assignment further includes assigning each of a second subset of the set of training gene-expression data elements to a second tumor-type class. For each training gene-expression data element of the second subset, the tumor was a non-neuronal and non-neuroendocrine tumor. A machine-learning model is trained using the set of training gene-expression data elements and the tumor-type class assignments. Training the machine-learning model includes learning a set of parameters. Based on the learned set of parameters, an incomplete subset of the set of genes is identified for which expression metrics are informative as to tumor-type class assignments. A specification for a gene panel for checkpoint-blockade-therapy amenability is output. The specification identifies each of one or more genes represented in the incomplete subset.

In some instances, the first subset can include an additional gene-expression data element generated based on another sample collected from another subject having a neuroendocrine tumor. Training the machine-learning model can include, for each gene of the set of genes, identifying a first expression-metric statistic for the first tumor-type class and identifying a second expression-metric statistic for the second tumor-type class, and, for each gene of the incomplete subset, a difference between the first expression-metric statistic and the second expression-metric statistic can exceed a predefined threshold. Training the machine-learning model can include learning a set of weights, and wherein the incomplete subset is identified based on the set of weights. The machine-learning model can use a classification technique, and the learned parameters can correspond to a definition of a hyperplane. The machine-learning model can include a gradient boosting machine. The method can further include: receiving first gene-expression data corresponding to the gene panel; determining, based on the first gene-expression data, that a first tumor corresponds to the first tumor-type class; outputting a first output identifying a combination therapy as a therapy candidate, the combination therapy including an initial chemotherapy and subsequent checkpoint blockade therapy; receiving second gene-expression data corresponding to the gene panel; determining, based on the second gene-expression data, that a second tumor corresponds to the second tumor-type class (e.g., each of the first tumor and the second tumor having been identified as a non-neuronal and non-neuroendocrine tumor and as corresponding to a same type of organ); and outputting a second output identifying a first-line checkpoint blockade therapy as a therapy candidate.

In some instances, a computer-implemented method is provided for using a machine-learning model for determining that a first-line checkpoint blockade therapy is a therapy candidate for a given subject. A machine-learning model is accessed that has been trained by performing a set of operations. The set of operations includes accessing a set of training gene-expression data elements corresponding to one or more subjects. Each training gene-expression data element of the set of training gene-expression data elements had been generated based on a sample collected from a corresponding subject of the one or more subjects having a tumor. Each training gene-expression data element of the set of training gene-expression data elements indicates, for each gene of a set of genes, an expression metric corresponding to the gene. The set of operations also includes assigning each of the set of training gene-expression data elements to a tumor-type class. The assignment includes assigning each of a first subset of the set of training gene-expression data elements to a first tumor-type class. The first subset includes a training gene-expression data element for which the tumor was a neuronal tumor. The assignment also includes assigning each of a second subset of the set of training gene-expression data elements to a second tumor-type class. For each training gene-expression data element of the second subset, the tumor was a non-neuronal and non-neuroendocrine tumor. The set of operations further includes training a machine-learning model using the set of training gene-expression data elements and the tumor-type class assignments. Training the machine-learning model includes learning a set of parameters. A gene-expression data element is accessed. The gene-expression data element was generated based on another biopsy of another tumor. The other gene-expression data element indicates, for each gene of at least some of the set of genes, another expression metric corresponding to the gene. The trained machine-learning model is executed using the other gene-expression data element. The execution generates a result indicating that the other tumor is of the second tumor-class type. In response to the result, an output can be output. The output identifies a first-line checkpoint blockade therapy as a therapy candidate.

In some instances, the first subset can include an additional gene-expression data element generated based on another sample collected from another subject having a neuroendocrine tumor. The machine-learning model can use a classification technique, and the learned parameters can correspond to a definition of a hyperplane. The machine-learning model can include a gradient boosting machine. The other tumor can correspond to a melanoma tumor. The method can further include accessing an additional gene-expression data element having been generated based on an additional biopsy of an additional tumor (e.g., the additional tumor being of associated with a same anatomical location as the other tumor, the other tumor being associated with a first subject, and the additional tumor being associated with a second subject); executing the trained machine-learning model using the additional gene-expression data element (the execution generating an additional result indicating that the additional tumor is of the first tumor-class type); and in response to the additional result, outputting an additional output identifying another therapy as a therapy candidate for the second subject. The other therapy can a combination therapy that can include a first-line chemotherapy and a subsequent checkpoint blockade therapy. The additional tumor can be a non-neuronal and non-neuroendocrine tumor.

In some instances, a computer-implemented method is provided for estimating whether a subject is amenable to a particular therapy approach. A gene-expression data element is accessed. The gene-expression data element was generated based on a sample collected from a subject having a non-neuronal and non-neuroendocrine tumor. The gene-expression data element indicates, for each gene of multiple genes, an expression metric corresponding to the gene. It is determined that the gene-expression data element corresponds to a neuronal genetic signature. A therapy approach is identified that includes an initial chemotherapy treatment and a subsequent checkpoint blockade therapy. An indication is output that the subject is amenable to the therapy approach.

In some instances, the multiple genes can include at least one of SV2A, NCAM1, ITGB6, SH2D3A, TACSTD2, C29orf33, SFN, RND2, PHLDA3, OTX2, TBC1D2, C3orf52, ANXA11, MSI1, TET1, HSH2D, C6orf132, RCOR2, CFLAR, IL4R, SHISA7, DTX2, UNC93B1, and FLNB. The multiple genes can include at least five of SV2A, NCAM1, ITGB6, SH2D3A, TACSTD2, C29orf33, SFN, RND2, PHLDA3, OTX2, TBC1D2, C3orf52, ANXA11, MSI1, TET1, HSH2D, C6orf132, RCOR2, CFLAR, IL4R, SHISA7, DTX2, UNC93B1, and FLNB. The method can further include accessing another gene-expression data element having been generated based on another sample collected from another subject having another non-neuronal and non-neuroendocrine tumor (the non-neuronal and non-neuroendocrine tumor can be in a particular organ of the subject, the other non-neuronal and non-neuroendocrine tumor can be in another particular organ of the other subject, and the particular organ and the other particular organ can be of a same type of organ); determining that the other gene-expression data element does not correspond to the neuronal genetic signature; identifying another therapy approach that includes a first-line checkpoint blockade therapy; and outputting an indication that the other subject is amenable to the other therapy approach. The method can further include determining the neuronal genetic signature by training a classification algorithm using a training data set that includes a set of training gene-expression data elements (e.g., where training gene-expression data element of the set of training gene-expression data elements can indicate, for each gene of at least the multiple genes, an expression metric corresponding to the gene) and labeling data that associates a first subset of the set of training gene-expression data elements with a first label indicative of a tumor having a neuronal property and that associates a second subset of the set of training gene-expression data elements with a second label indicative of a tumor not having the neuronal property.

In some instances, a kit is provided for detecting gene expressions indicative of whether tumors are neurally related including a set of primers. Each primer of the set of primers can bind to a gene listed in Table 1, and the set of primers can include at least 5 primers.

In some instances, each of the set of primers can include an upstream primer, and the kit can further include a corresponding set of downstream primers. The set of primers includes at least 10 primers or at least 20 primers. For each of the set of primers, the gene to which the primer binds can be associated, in Table 1, with a weight above 5.0. For each of the set of primers, the gene to which the primer binds can be associated, in Table 1, with a weight above 1.0. For each of the set of primers, the gene to which the primer binds can be associated, in Table 1, with a weight above 0.5.

In some instances, a system is provided that includes one or more data processors and a non-transitory computer readable storage medium containing instructions which, when executed on the one or more data processors, cause the one or more data processors to perform part or all of one or more methods disclosed herein.

In some instances, a computer-program product is provided that is tangibly embodied in a non-transitory machine-readable storage medium. The computer-program product can include instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 shows effector T cell levels in samples from different types of tumors;

FIG. 2 shows an computing system for using a machine-learning model to identify results facilitating tumor categorization;

FIG. 3 shows exemplary mappings for data labeling and uses thereof;

FIG. 4 shows training-data and test-data results generated using a trained machine-learning model;

FIG. 5 illustrates a degree to which, for different tumor categories (rows), subsets corresponding to different ML-generated categories differ with respect to identified immune and stromal-infiltration signatures (columns);

FIGS. 6A-6F show clinical data, separated by categories generated by a trained machine-learning model;

FIG. 7 shows clinical data, separated by categories generated by a trained machine-learning model;

FIG. 8 shows exemplary Kaplan-Meier curves for different proliferation and neurally related classes;

FIGS. 9A-9C show data, separated by categories pertaining to being neurally related (or not), stemlike (or not) and/or proliferation (low or high);

FIG. 10 shows immune-cell signatures and mutation statistics for neuroendocrine and non-neuroendocrine data cohorts;

FIG. 11 shows expression levels for six neuronal/neuroendocrine marker genes across samples for different types of tumors;

FIG. 12 shows scores of various neuronal/neuroendocrine gene signatures across samples for different types of tumors;

FIG. 13A shows the first and second principal components across samples for different types of tumors when a PCT-based approach was used to process gene-expression data;

FIG. 13B shows the third, fourth, fifth and sixth principal components across samples for different types of tumors when a PCT-based approach was used to process gene-expression data;

FIG. 14 shows, for individual types of tumors, principal component values generated for neurally related samples and for non-neurally related samples;

FIG. 15 shows scores, generated by a classifier, corresponding to predictions as to whether various gene-expression data sets correspond to a neurally related class;

FIG. 16 shows a degree to which expression levels of various genes were important with regard to influencing neurally related classifications;

FIG. 17 shows representations as to how expression of various genes differed between neurally related tumors and non-neurally related tumors;

FIG. 18 shows which a breakdown of the types of tumors represented in tumors predicted to be neurally related by a classifier model;

FIG. 19 shows Uniform Manifold Approximation and Projection (UMAP) projections for various samples and tumor types;

FIG. 20 shows adjusted p-values when comparing UMAP values corresponding to tumors from the holdout set that were predicted to be neurally related with UMAP values corresponding to tumors from the training set that were predicted to be neurally related;

FIG. 21 shows, for each of two genes and each of two tumor types, classifier scores corresponding to predictions as to whether various samples are neurally related, separated based on whether the sample included a mutation of the gene;

FIG. 22 shows, for each of multiple melanoma subtypes, scores predicting neural relatedness and stemness scores;

FIG. 23 illustrates a process of using a machine-learning model to identify a panel specification;

FIG. 24 illustrates a process of using a machine-learning model to identify therapy-candidate data; and

FIG. 25 illustrates a process of identifying a therapy amenability based on a neural-signature analysis.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

### I. Overview

Cancer immunotherapy harnesses aspects of a subject's own immune system in order to slow, stop, or reverse tumor growth. Some immunotherapies are designed to adjust the activity of T-cells, which mediate cell death of diseased or damaged cells within the subject. For example, checkpoint proteins are native components of the human immune system, and some act to inhibit T-cell activity. In normal circumstances, this inhibition can prevent extended attacks on self that would lead to inflammatory tissue damage and/or autoimmune disease. However, some tumors also produce checkpoint proteins such that the tumor is protected from T-cells that would otherwise be effective in killing tumor cells. Checkpoint inhibitor therapy is a type of cancer immunotherapy designed to block checkpoint proteins, so that the body's own T-cells can better act to kill tumor cells.

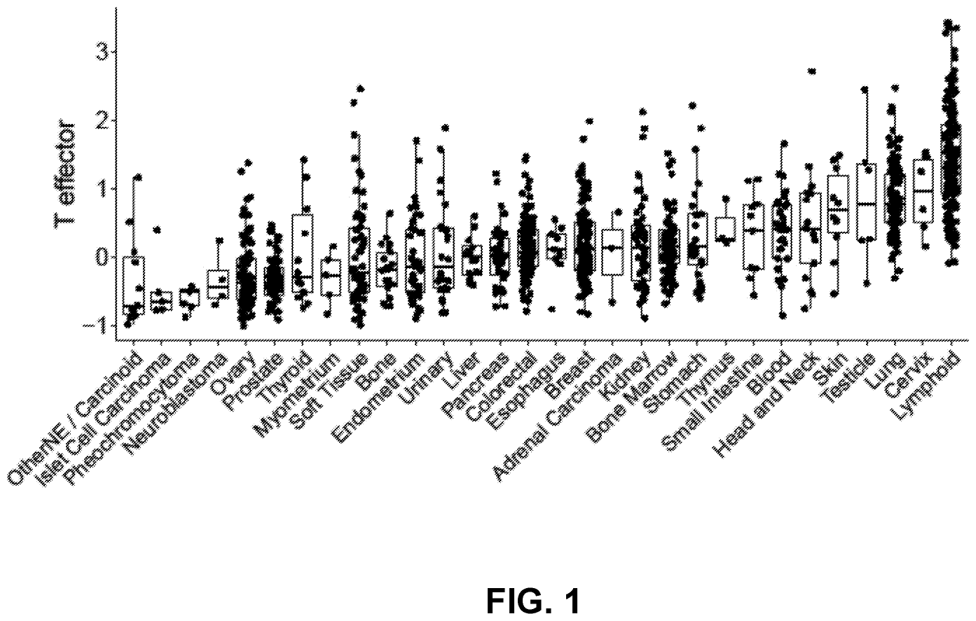

However, checkpoint inhibitor therapy will only increase T-cell activity within a tumor if the body's T-cells were already present in sufficient numbers to affect the tumor, which itself depends on whether the subject's immune system has responded to the presence of the tumor by creating T-cells to attack it. FIG. 1 shows how levels of effector T cells vary across tumor types and samples (with each point representing a sample). High levels of effector T cells are indicative of a large immune response. Notably, while marked differences in effector T cells are present across tumor types, the range of these levels is highly overlapping across tumor types. The wide range of effector T cell levels across samples in each given type of tumor and the high overlap of effector T cell levels across tumor types suggests that tumor type alone is insufficient to indicate whether a subject's immune system is activated and whether checkpoint blockade therapy is likely to be an effective treatment.

Whether or not the subject's immune system activates in this regard in response to the presence of the tumor can depend on the tumor's immune phenotype. Tumors can be categorized as being immunologically “hot” or immunologically “cold” in this regard. A cold tumor (or “immune desert” tumor) is one that is uninflamed and showing no immune cell infiltration. More specifically, a tumor may remain undetected, such that only a weak T-cell immune response or no T-cell immune response is elicited to attack the tumor. Meanwhile, a hot tumor (or “inflamed” tumor) is one that has pronounced T-cell infiltration in the tumor core. Thus, a tumor may be classified as either a hot tumor or cold tumor based on expression of T-cell markers (such that a tumor is designated as a hot tumor when the marker(s) is indicative of a T-cell-inflamed phenotype).

In some approaches, checkpoint blockade therapy may be selectively identified as a first-line therapy when tumor is hot. However, tumors can be characterized using other properties, and thus, it is possible that stratifying tumors in a different manner may be alternatively or further predictive as to whether checkpoint blockade therapy would be an effective treatment. One approach disclosed herein relates to characterizing a tumor as one of a neurally related (or neural) tumor or a non-neurally related (or non-neural) tumors. A neural characterization may (but need not) indicate that the tumor has a neural embryonic origin, such as the neural crest. Neurally related tumors can include brain tumors and neuroendocrine tumors, though this list is under-inclusive, in that at least some tumors of other types may be neurally related.

In some embodiments, a machine-learning model is provided that uses gene expression data to estimate whether a tumor is neurally related. More specifically, in some instances, a machine-learning model can be trained using a training data set that includes a set of positive data elements (corresponding to a first class) and a set of negative data elements (corresponding to a second class). Each of the sets of positive and negative elements can include data that indicate, for each of a set of genes, expression data. This expression data may be represented in the form of RNA transcript counts (or abundance estimates) as determined from next generation sequencing, a processed version thereof (e.g., by normalizing the transcript count across the entire set of measured genes, calculating a log of the transcript count, or determining a normalized log-transformed value of RNA-Seq data). In some instances, each of the set of positive data elements corresponds to a brain tumor or a neuroendocrine tumor. In some instances, each of the set of negative data elements corresponds to a tumor that is not a brain tumor and is not a neuroendocrine tumor.

Training the machine-learning model can include learning (for example) gene-associated weights, gene expression characteristics and/or signatures for each of the neurally related and non-neurally related data sets. The learned data can be used to identify a subset of genes for which expression data is informative and/or predictive of a class assignment for the tumor being neurally related or not. each of the subset of genes may have been associated with weights and/or significance values that exceed an absolute or predefined threshold (e.g., so as to identify a predefined number of genes associated with the highest weights across a gene set, so as to identify each gene from a gene set associated with a weight exceeding a predefined threshold, etc.).

A result may be generated and output (transmitted and/or presented) that indicates a specification for a gene panel may identify the subset of genes. A gene panel may then be designed and implemented accordingly, such that its results identify expression of and/or any mutations in each of the subset of genes. More specifically, a gene panel may be designed to use particular primers or probes to bind to sites near and/or within the subset of genes. Each primer and/or probe can include a label. In some instances, a prevalence of the label(s) relative to a prevalence of other markers associated with other genes can indicate an expression of the gene. In some instances, an order in which different labels are detected can identify an actual primary sequence of the gene, which can then be compared to a reference sequence to determine whether a subject has any mutations in relation to the gene.

A result produced by the machine-learning model may indicate whether, an extent to which and/or how expression of each of a set of genes is predictive of a category assignment (e.g., that associates a sample with a neurally related or non-neurally related category). For example, a binary indication may indicate that any expression or high expression of a given gene is associated with or correlated with assignment to a class of a given category (e.g., a neurally related class or a non-neurally related class). As another example, a numeric indication may indicate an extent to which expression of a given gene is associated with or correlated with assignment to a class, with negative numbers representing an association with one category and positive numbers representing an association with another category.

In some instances, expression data corresponding to a given subject is input into the trained machine-learning model. Execution of the trained machine-learning model can result in generating a category that corresponds to an estimate as to whether a tumor of the subject's is neurally related. The result may include or represent a degree of confidence of the estimation. It will be appreciated that identities of genes represented in the input expression data need not be the same as identities of genes represented in the training data. The trained machine-learning model may then generate a result based on at least some of the genes represented both in the training data and in the input expression data. In some instances, a result that is output may represent or include a category. In some instances, a result further or alternatively identifies a candidate treatment, which may be selected based on an assigned category. For example, a checkpoint blockade therapy may be identified as a candidate for a first-line therapy when an assigned category estimates that a tumor does not correspond to a neural signature and/or does not correspond to a neurally related class. Meanwhile, an alternative therapy approach (e.g., an initial chemotherapy treatment followed by a checkpoint blockade therapy) may be identified as a candidate when an assigned category estimates that a tumor corresponds to a neural signature and/or corresponds to a neurally related class. In some instances, a result that is output includes or represents a prediction (made based on a category assigned to a particular input data set corresponding to a subject) as to whether a particular treatment approach would be effective in treating a medical condition (e.g., at slowing, stopping and/or reversing progression of a cancer in the subject). In some instances, a result identifies or indicates a particular treatment approach (e.g., checkpoint blockade therapy as a first-line treatment approach when an input data set is assigned to a neurally related category).

In some instances, a kit is designed and provided. The kit may include primers and/or probes configured to facilitate detecting expression and/or mutations corresponding to neurally related genes. The kit can further include such primers and/or probes fixed to a substrate. The kit can further include a microarray.

### II. Definitions and Abbreviations

As used herein, the term “neurally related” tumor (or tumor cell) refers to a tumor (or tumor cell) having a molecular profile that is more similar to molecular profiles of tumor cells of a neural embryonic origin (e.g., cell lineages traceable back to the neural crest or the neural tube, including both central nervous system and neuroendocrine cell types) relative to molecular profiles of tumor cells not having a neural embryonic origin. Some embodiments of the invention relate to determining treatment recommendations, determining treatments and/or treating a subject based on whether one or more tumors of the subject are neurally related. Tumors cells with neural embryonic origin include cells from a brain tumor (e.g., glioblastoma and glioma), from some neuroendocrine tumors (e.g., pheochromocytoma, paraganglioma). Neurally related tumors also include neuroendocrine tumors, (including neuroendocrine tumors that develop from non-neural crest derived tissues, such as pancreatic neuroendocrine tumor, and lung adenocarcinoma—large cell neuroendocrine tumor) and from other neurally related tumors (e.g., muscle-invasive bladder cancer—expression based neuronal subtype). Tumor cells not having a neural embryonic origin can include non-neuroendocrine cells from a tumor that is not in the brain (e.g., cells from pancreatic ductal adenocarcinoma, non-neuroendocrine lung adenocarcinoma and non-neuroendocrine muscle-invasive bladder cancer). Non-neuroendocrine tumors that are not in the brain may include one or more neurally related tumor cells that have molecular profiles more similar to (e.g., as determined based on an output of a classifier) molecular profiles of tumor cells of a neural embryonic origin than molecular profiles of tumor cells not having a neural embryonic origin. For example, a classifier may output a prediction that particular molecular-profile data corresponds to a class associated with neural embryonic origin (e.g., a binary indicator, a confidence of such classification that exceeds a predefined threshold and/or a predicted probability of such classification that exceeds a predefined threshold). Neurally related tumors (or tumor cells) may arise in non-neuroendocrine tumors that are not in the brain as a result of particular microenvironments and/or biological experiences. For example, a neurally related tumor cell may arise due to drug resistance mechanisms and/or due to a tumor adapting to a microenvironment by including tumor cells having molecular profiles more similar to molecular profiles of tumor cells of a neural embryonic origin than of tumor cells not having a neural embryonic origin.

As used herein, the term “non-neurally related” tumor (or tumor cell) refers to a tumor (or tumor cell) having a molecular profile that is more similar to molecular profiles of tumor cells not having a neural embryonic origin relative to molecular profiles of tumor cells having a neural embryonic origin.

As used herein, the term “gene panel” refers to a group of one or more probes or primers used to identify the presence and/or amount of one or more selected nucleic acids of interest, for example, one or more DNA or RNA sequences of interest. The specific primers or probes can be selected for a specific function (e.g., for detection of nucleic acids associated with a specific type of neural disease or trait) or can be selected for whole genome sequencing. Oligonucleotide probes and primers can be about 20 to about 40 nucleotide residues in length. The primers or probes can be detectably labeled or the product thereof is detectably labelled. Detectable labels include radionuclides, chemical moieties, fluorescent moieties, and the like. The probe or primer can include a fluorescent label and a fluorescence-quenching moiety whereby the fluorescent signal is reduced when the two bind to a nucleic acid of interest in close proximity. Molecular beacon systems can be used. Multiple detectable labels can be used in multiplex assay systems. The gene panel can be a microarray. A gene panel can be designed to identify mutations or alleles by (for example) detecting positive (inclusion of the mutation or allele) or negative (exclusion of the mutation or allele) results. The gene panel can be “read” using nucleic acid sequencing using sequencing methods known to one of ordinary skill in the art. Exemplary sequencing methods and systems include, but are not limited to, Maxam-Gilbert sequencing, dye-terminator sequencing, Lynx Therapeutics' Massively Parallel Sequencing (MPSS) Polony sequencing, 454 Pyrosequencing, Illumina (Solexa) sequencing, SOLiD™ sequencing, Single Molecule SMART sequencing, Single Molecule real time (RNAP) sequencing, and Nanaopore DNA sequencing.

As used herein, the term “probe” refers to an oligonucleotide that hybridizes with a nucleic acid of interest, but the term also includes reagents used in new generation nucleic acid sequencing technologies. The probe need not hybridize to a location that includes the mutation or allelic site, but can upstream (5′) and/or downstream (3′) of the mutation or allele.

As used herein, the term “primer” refers to an oligonucleotide primer that initiates a sequencing reaction performed on a selected nucleic acid. A primer can include a forward sequencing primer and/or a reverse sequencing primer. Primers or probes in a gene panel can be bound to a substrate or unbound. Alternatively, one or more primers can be used to specifically amplify at least a portion of a nucleic acid of interest. mRNA transcripts can be reverse transcribed to generate a cDNA library prior to amplification. A detectably labeled polynucleotide capable of hybridizing to the amplified portion can be used to identify the presence and/or amount of one or more selected nucleic acids of interest.

As used herein, a “subject” encompasses one or more cells, tissue, or an organism. The subject may be a human or non-human, whether in vivo, ex vivo, or in vitro, male or female. A subject can be a mammal, such as a human.

As used herein, the term “gene-expression data element” refers to data indicating one or more genes are expressed in a sample or subject. A gene-expression data element may identify which genes are expressed in a sample or subject and/or a quantitative expression level of each of one or more genes. Gene expression may be determined by (for example) measuring mRNA levels (e.g., via next-generation sequencing, microarray analysis or reverse transcription polymerase chain reaction) or measuring protein levels (e.g., via a Western blot or immunohistochemistry)

As used herein, the term “checkpoint-blockade-therapy amenability” refers to a prediction as to whether checkpoint blockade therapy (e.g., when used as an initial therapy and/or without a preceding chemotherapeutic therapy) will slow progression of cancer and/or reduce the size of one or more tumors in a given subject.

As used herein, the term “neuronal genetic signature” (also referred to herein as “neural signature”) refers to data that identifies particular genes that are expressed in neurally related tumors and/or expression levels (e.g., expression-level statistics and/or expression-level ranges) of particular genes in neurally related tumors. A neuronal genetic signature may identify genes (and/or expression levels thereof) that are (e.g., typically, generally or always) expressed in neurally related tumors and not (e.g., typically, generally or always) expressed in non-neurally related tumors. A neuronal genetic signature may identify genes (and/or expression levels thereof) that are (e.g., typically, generally or always) more highly expressed in neurally related tumors as compared to non-neurally related tumors. A neuronal genetic signature may comprise a set of genes that have been identified as informative of assignment to one of a first class of tumors comprising one or more neuronal tumors and optionally one or more neuroendocrine tumors, and a second class of tumors comprising one or more tumors that are each non-neural and non-neuroendocrine, as described herein.

As used herein, the term “checkpoint blockade therapy” refers to an immunotherapy that includes immune checkpoint inhibitors. Each of the one or more immune checkpoint inhibitors targets immune checkpoints, which are proteins that regulate (e.g., inhibit) immune responses. Exemplary checkpoints include PD-1/PD-L1 and CTLA-4/B7-1/7-2. Select abbreviations pertinent to disclosures herein include:

| Abbreviation | Name |
|---|---|
| LAML | Acute Myeloid Leukemia |
| ACC | Adrenocortical carcinoma |
| BLCA | Bladder Urothelial Carcinoma |
| LGG | Brain Lower Grade Glioma |
| BRCA | Breast invasive carcinoma |
| CESC | Cervical squamous cell carcinoma and endocervical |
| | adenocarcinoma |
| CHOL | Cholangiocarcinoma |
| LCML | Chronic Myelogenous Leukemia |
| COAD | Colon adenocarcinoma |
| CNTL | Controls |
| ESCA | Esophageal carcinoma |
| FPPP | FFPE Pilot Phase II |
| GSM | Glioblastoma multiforme |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LIHC | Liver hepatocellular carcinoma |
| LUAD | Lung adenocarcinoma |
| LUSC | Lung squamous cell carcinoma |
| DLBC | Lymphoid Neoplasm Diffuse Large B-cell Lymphoma |
| MESO | Mesothelioma |
| MISO | Miscellaneous |
| OV | Ovarian serous cystadenocarcinoma |
| PAAD | Pancreatic adenocarcinoma |
| PCPG | Pheochromocytoma and Paraganglioma |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THYM | Thymoma |
| THCA | Thyroid carcinoma |
| UCS | Uterine Carcinosarcoma |
| UCEC | Uterine Corpus Endometrial Carcinoma |
| UVM | Uveal Melanoma |

### III. Computing Environment and Model Architecture

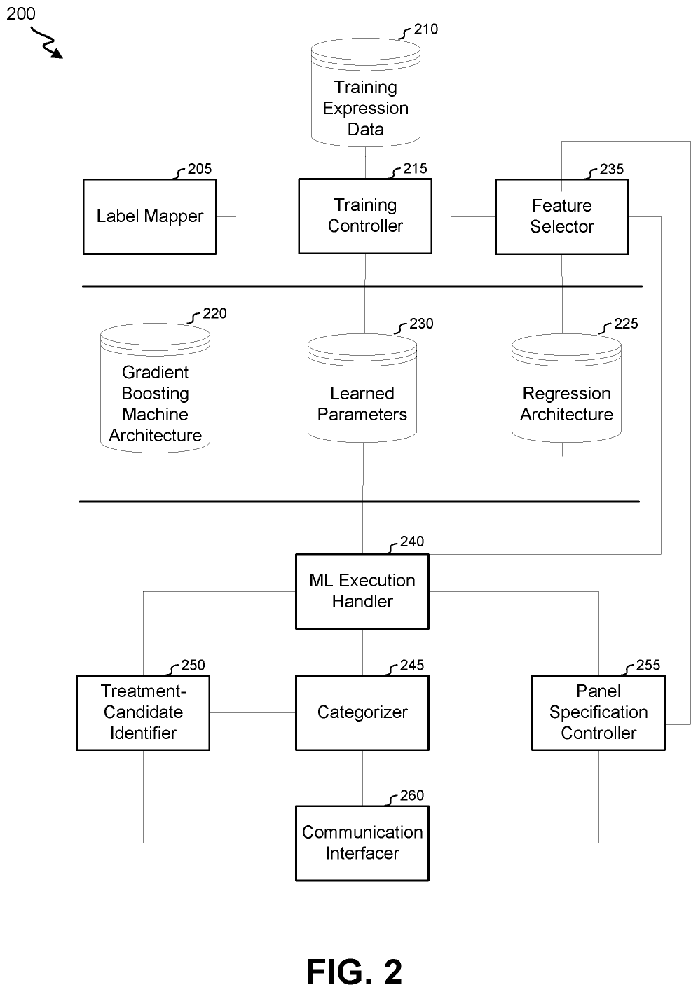

FIG. 2 shows an computing system 200 for training and using a machine-learning model to identify results facilitating tumor categorizations. Computing system 200 includes a label mapper 205 that maps particular sets of tumors to a “neurally related” label (e.g. assign a “neurally related” label to particular types of tumors) and that maps other particular sets of tumors to a “non-neurally related” label. The particular sets of tumors can include brain tumors and/or neuroendocrine tumors. In some instances, each of the other particular sets of tumors is not a brain tumor and not a neuroendocrine tumor. The mapping need not be exhaustive. For example, the mapping may be reserved to apply to sets of tumors for which there is high confidence and/or certainty as to whether the tumor is a brain tumor, is a neuroendocrine tumor and/or corresponds to a neural signature, such that other tumors may have no label at all.

Mapping data may be stored in a mapping data store (not shown). The mapping data may identify each tumor that is mapped to either of the neurally related label or the non-neurally related label. The mapping data may (but need not) further identify additional sets of tumors (e.g., that may be or have the potential to be associated with either label).

A training expression data store 210 can store training gene-expression data for each of one or more sets of tumors (including some or all of those mapped to the neurally related label and non-neurally related label). The training gene-expression data may include (for example) RNA-Seq data. The training gene-expression data stored in training expression data store 210 may have been collected (for example) from a public data store and/or from data received from (for example) a lab or physician's office.

To obtain RNA-Seq data, RNA can be isolated from tissue and combined with deoxyribonuclease (DNase) to decrease the quantity of genomic DNA and thus provide isolated RNA. The isolated RNA may be filtered (e.g., with poly(A) tails) to filter out rRNA and produce isolated mRNA, may be filtered for RNA that bind to particular sequences and/or left in its original isolated state. The RNA (or mRNA or filtered RNA) can be reverse transcribed to cDNA, which can then be sequenced typically using next generation sequencing technologies. Direct (or “bulk”) RNA sequencing or single-cell RNA sequencing can be performed to generate expression profiles. Transcription assembly can then be performed (e.g., using a de novo approach or alignment with a reference sequence), and expression data can be generated by counting a number of reads aligned to each locus and/or transcript, and/or by obtaining an estimate of the abundance of one or more gene expression products using such counts. The RNA-Seq data can be defined to include this expression data.

Training controller 215 can use the mappings and a training gene-expression data set to train a machine-learning model. More specifically, training controller 215 can access an architecture of a model, define (fixed) hyperparameters for the model (which are parameters that influence the learning process, such as e.g. the learning rate, size/complexity of the model, etc.), and train the model such that a set of parameters are learned. More specifically, the set of parameters may be learned by identifying parameter values that are associated with a low or lowest loss, cost or error generated by comparing predicted outputs (obtained using given parameter values) with actual outputs. In some instances, a machine-learning model includes a gradient boosting machine or regression model (e.g., linear regression model or logistic regression model, which may implement a penalty such as an L1 penalty). Thus, training controller 215 may retrieve a stored gradient boosting machine architecture 220 or a stored regression architecture 225. A gradient boosting machine can be configured to iteratively fit new models to improve estimation accuracy of an output (e.g., that includes a metric or identifier corresponding to an estimate or likelihood as to whether a tumor is neurally related or not). The new base-learners can be constructed to optimize correlation with the negative gradient of the loss function of the whole ensemble. Thus, gradient boosting machines may rely upon a set of base learners, each of which may have their own architecture (not shown). Gradient boosting machines may be advantageous to use, in that, in an external data set that does not include expression data for some genes, the model can still generate an output using only expression data for available genes. Another approach (for example, with respect to a logistic regression) is to impute missing expression data. A regression model may be more simplistic and faster, though it may then introduce biases.

Learned parameters can include (for example) weights. In some instances, each of at least one of the weights corresponds to an individual gene, such that the weight may indicate a degree to which expression of the individual gene is informative as to a label of a tumor. In some instances, each of at least one weight corresponds to multiple genes.

A feature selector 235 can use data collected throughout training and/or learned parameters to select a set of features that are informative of a result of interest. For example, an initial training may be conducted to concurrently or iteratively evaluate how expression data for hundreds or thousands of genes relates to a result (e.g., tumor categorization label). Feature selector 235 can then identify an incomplete subset of the hundreds or thousands of genes, such that each gene within the subset is associated with a metric (e.g., significance value and/or weight value) that exceeds a predefined absolute or relative threshold. For example, feature selector 235 may identify 5, 10, 15, 20, 25, 50, 100, or any other number of genes that are most informative of a label. In some instances, feature selector 235 and training controller 215 coordinate such that trainings are iteratively performed using different training expression data sets (corresponding to different genes) based on feature-selection results. For example, an initial set of genes may be iteratively and repeatedly filtered to arrive at a set that are informative as to a tumor's label.

A set of features selected by feature selector 235 can correspond to (for example) at least 1, at least 5, at least 10, at least 15, at least 20, at least 25 or at least 50 genes identified in Table 1. A set of features can include (for example) at least 1, at least 5, at least 10 or at least 20 genes associated with (in Table 1) a weight that is above 1.0, 0.75, 0.5 or 0.25. A set of features can include (for example) at least 25, at least 50 or at least 100 genes associated with (in Table 1) a weight that is above 0.25, 0.1, 0.1 or 0.05.

TABLE 1

| Weight | Entrez gene ID | Gene symbol |
|---|---|---|
| 360.445279772379 | 9900 | SV2A |
| 249.997221599892 | 4684 | NCAM1 |
| 205.827480315269 | 3694 | ITGB6 |
| 121.855306294844 | 10045 | SH2D3A |
| 101.857028317821 | 4070 | TACSTD2 |
| 101.476924156931 | 64073 | C19orf33 |
| 65.4941865211253 | 2810 | SFN |
| 13.5112481165347 | 8153 | RND2 |
| 6.49000529393473 | 23612 | PHLDA3 |
| 6.41967549031834 | 5015 | OTX2 |
| 5.47443789767072 | 55357 | TBC1D2 |
| 4.55850605530984 | 79669 | C3orf52 |
| 4.46658281616014 | 311 | ANXA11 |
| 3.60026148965694 | 4440 | MSI1 |
| 3.36488265134306 | 80312 | TET1 |
| 3.14541492638473 | 84941 | HSH2D |
| 2.69478248394918 | 647024 | C6orf132 |
| 2.39272745313085 | 283248 | RCOR2 |
| 2.04598900873111 | 8837 | CFLAR |
| 2.01802936086326 | 3566 | IL4R |
| 1.99517806987709 | 729956 | SHISA7 |
| 1.95489481668363 | 113878 | DTX2 |
| 1.78304989560797 | 81622 | UNC93B1 |
| 1.65734283541514 | 2317 | FLNB |
| 1.32934155209402 | 22844 | FRMPD1 |
| 1.30607849207192 | 387104 | C6orf174 |
| 1.2488652149452 | 55964 | SEPT3 |
| 1.23525941876557 | 79570 | NKAIN1 |
| 1.10861451834804 | 199731 | CADM4 |
| 1.10825044427341 | 51560 | RAB6B |
| 1.05254445353258 | 55028 | C17orf80 |
| 1.03917187229879 | 3383 | ICAM1 |
| 1.03112846814804 | 547 | KIF1A |
| 1.01293392881864 | 57501 | KIAA1257 |
| 0.990376069267529 | 56896 | DPYSL5 |
| 0.989982864114279 | 134111 | UBE2QL1 |
| 0.927751353776179 | 122060 | SLAIN1 |
| 0.895309609190046 | 66000 | TMEM108 |
| 0.883441782678008 | 5455 | POU3F3 |
| 0.87881900552203 | 119 | ADD2 |
| 0.840351939613452 | 9355 | LHX2 |
| 0.767682807589544 | 8577 | TMEFF1 |
| 0.755092744848901 | 837 | CASP4 |
| 0.752981108462977 | 401431 | LOC401431 |
| 0.74439531056218 | 9633 | MTL5 |
| 0.722496794270635 | 6328 | SCN3A |
| 0.697062549130178 | 118430 | MUCL1 |
| 0.693209621674173 | 80736 | SLC44A4 |
| 0.6879941888413 | 84206 | MEX3B |
| 0.686928449723438 | 116984 | ARAP2 |
| 0.674902133069499 | 8927 | BSN |
| 0.654902780288967 | 1496 | CTNNA2 |
| 0.644361475038555 | 1804 | DPP6 |
| 0.598646837145586 | 575 | BAI1 |
| 0.591136824052263 | 23532 | PRAME |
| 0.590165469739351 | 60370 | AVPI1 |
| 0.584407840964803 | 253724 | LOC253724 |
| 0.583330652854451 | 255189 | PLA2G4F |
| 0.583174159126591 | 1728 | NQO1 |
| 0.572952362767678 | 79658 | ARHGAP10 |
| 0.559849622420237 | 64855 | FAM129B |
| 0.541217866239475 | 10198 | MPHOSPH9 |
| 0.538046296113624 | 823 | CAPN1 |
| 0.5148463088355 | 113730 | KLHDC7B |
| 0.511784827948473 | 861 | RUNX1 |
| 0.49269556749652 | 51286 | CEND1 |
| 0.490276243815408 | 114043 | C21orf90 |
| 0.468293011829404 | 9121 | SLC16A5 |
| 0.460680110147172 | 50804 | MYEF2 |
| 0.448824277555329 | 81796 | SLCO5A1 |
| 0.445456957927441 | 8869 | ST3GAL5 |
| 0.425401917041082 | 4131 | MAP1B |
| 0.425104413547099 | 59272 | ACE2 |
| 0.407829168408829 | 1000 | CDH2 |
| 0.401335025753988 | 79012 | CAMKV |
| 0.400663306167484 | 140690 | CTCFL |
| 0.394532953636824 | 78986 | DUSP26 |
| 0.392052567815578 | 4109 | MAGEA10 |
| 0.391951710261555 | 956 | ENTPD3 |
| 0.384756769271621 | 4600 | MX2 |
| 0.373835701478419 | 89874 | SLC25A21 |
| 0.369955185400179 | 84733 | CBX2 |
| 0.353470856090267 | 116442 | RAB39B |
| 0.353445139431446 | 344167 | FOXI3 |
| 0.352564743418233 | 11281 | POU6F2 |
| 0.345739182663362 | 155066 | ATP6V0E2 |
| 0.341272646437358 | 9468 | PCYT1B |
| 0.340553515177491 | 9427 | ECEL1 |
| 0.32802601392343 | 6620 | SNCB |
| 0.318546996998454 | 51252 | FAM178B |
| 0.314473930098019 | 2863 | GPR39 |
| 0.312067499214539 | 55808 | ST6GALNAC1 |
| 0.302305399354751 | 79977 | GRHL2 |
| 0.289611254746009 | 4101 | MAGEA2 |
| 0.28821914806661 | 54549 | SDK2 |
| 0.287715407421836 | 1627 | DBN1 |
| 0.279328096263628 | 80830 | APOL6 |
| 0.273926659613489 | 796 | CALCA |
| 0.271463018997601 | 25806 | VAX2 |
| 0.266098159708447 | 3880 | KRT19 |
| 0.251930269809281 | 10550 | ARL6IP5 |
| 0.248899378149814 | 65267 | WNK3 |
| 0.240303622772706 | 57823 | SLAMF7 |
| 0.234040759160452 | 84457 | PHYHIPL |
| 0.23124026753697 | 26579 | MYEOV |
| 0.225280515882785 | 401491 | FLJ35024 |
| 0.220581177283387 | 56140 | PCDHA8 |
| 0.213867598233432 | 23025 | UNC13A |
| 0.209107554197927 | 3475 | IFRD1 |
| 0.203469933124702 | 2260 | FGFR1 |
| 0.200243995471846 | 54961 | SSH3 |
| 0.198456213906412 | 8936 | WASF1 |
| 0.192611493209613 | 79148 | MMP28 |
| 0.190869276405472 | 51702 | PADI3 |
| 0.190761090065185 | 81693 | AMN |
| 0.18954644789091 | 375323 | LHFPL4 |
| 0.187377930711425 | 244 | ANXA8L2 |
| 0.18125003420493 | 4909 | NTF4 |
| 0.179467661587543 | 50861 | STMN3 |
| 0.177979223449185 | 121506 | ERP27 |
| 0.173087844016674 | 7976 | FZD3 |
| 0.170567383446748 | 6041 | RNASEL |
| 0.161019644223342 | 64927 | TTC23 |
| 0.155017796640191 | 54328 | GPR173 |
| 0.153889868803509 | 7846 | TUBA1A |
| 0.149331845237162 | 202052 | DNAJC18 |
| 0.149287066437414 | 4857 | NOVA1 |
| 0.148469600011229 | 2867 | FFAR2 |
| 0.143782806008358 | 117854 | TRIM6 |
| 0.143293559267671 | 153769 | SH3RF2 |
| 0.140865229296159 | 440362 | HERC2P4 |
| 0.140013488107983 | 6707 | SPRR3 |
| 0.137512036976695 | 23208 | SYT11 |
| 0.135775839628469 | 8399 | PLA2G10 |
| 0.134917281063397 | 22837 | COBLL1 |
| 0.134850688736163 | 92840 | REEP6 |
| 0.127674318316024 | 254263 | CNIH2 |
| 0.125456734078219 | 6285 | S100B |
| 0.120785270541444 | 25999 | CLIP3 |
| 0.118914970750324 | 731789 | LOC731789 |
| 0.118733432704482 | 6928 | HNF1B |
| 0.117188409762111 | 3691 | ITGB4 |
| 0.115324574646174 | 130612 | TMEM198 |
| 0.112319043926867 | 874 | CBR3 |
| 0.111278754920851 | 83723 | FAM57B |
| 0.104613827372456 | 1995 | ELAVL3 |
| 0.103137491597325 | 2775 | GNAO1 |
| 0.10082645311559 | 57578 | KIAA1409 |
| 0.0991990401826142 | 100190940 | LOC100190940 |
| 0.0970932085266611 | 161253 | REM2 |
| 0.0944949048278536 | 10389 | SCML2 |
| 0.0943655448182787 | 54933 | RHBDL2 |
| 0.0875382624538638 | 159963 | SLC5A12 |
| 0.0853277411487715 | 29763 | PACSIN3 |
| 0.0848026751612597 | 4897 | NRCAM |
| 0.0845272857378767 | 5629 | PROX1 |
| 0.0831722579244069 | 8717 | TRADD |
| 0.0787036548511575 | 100131726 | LOC100131726 |
| 0.0786764315052387 | 84069 | PLEKHN1 |
| 0.0767193523212458 | 94234 | FOXQ1 |
| 0.0746921780224602 | 55034 | MOCOS |
| 0.0744206120159156 | 7098 | TLR3 |
| 0.0734962292507258 | 7498 | XDH |
| 0.0729368688510757 | 4133 | MAP2 |
| 0.071928038427034 | 80821 | DDHD1 |
| 0.0665765921464937 | 8942 | KYNU |
| 0.065738247463551 | 777 | CACNA1E |
| 0.0650083876000417 | 4907 | NT5E |
| 0.0634360783419668 | 6277 | S100A6 |
| 0.0629607494753856 | 60680 | CELF5 |
| 0.0616852676878791 | 22843 | PPM1E |
| 0.0603040161793494 | 730101 | LOC730101 |
| 0.0593408199485206 | 347735 | SERINC2 |
| 0.0530484807893393 | 54677 | CROT |
| 0.0510163288689371 | 64781 | CERK |
| 0.0507021377867923 | 10297 | APC2 |
| 0.0500683519960521 | 84951 | TNS4 |
| 0.0489733634193337 | 26074 | C20orf26 |
| 0.0479095771705111 | 10509 | SEMA4B |
| 0.0468631650532265 | 1641 | DCX |
| 0.0466996600282329 | 64129 | TINAGL1 |
| 0.0465809830028973 | 55107 | ANO1 |
| 0.0461186944818273 | 5274 | SERPINI1 |
| 0.045679348302271 | 3248 | HPGD |
| 0.0453309244903127 | 9180 | OSMR |
| 0.0445406882200167 | 4137 | MAPT |
| 0.0439205412277818 | 5979 | RET |
| 0.043531647482433 | 5029 | P2RY2 |
| 0.0417938653792122 | 2525 | FUT3 |
| 0.0410329320165784 | 9911 | TMCC2 |
| 0.0409393152325187 | 25833 | POU2F3 |
| 0.0398377287186295 | 333 | APLP1 |
| 0.0382943929971389 | 8531 | CSDA |
| 0.0381408217771491 | 1510 | CTSE |
| 0.0377517641077318 | 64859 | OBFC2A |
| 0.0358582377210745 | 10512 | SEMA3C |
| 0.0347201943844781 | 2897 | GRIK1 |
| 0.0332412444735184 | 9108 | MTMR7 |
| 0.0314887941673368 | 7476 | WNT7A |
| 0.0314168608456708 | 81027 | TUBB1 |
| 0.0307394770353346 | 10610 | ST6GALNAC2 |
| 0.030566204214459 | 84894 | LINGO1 |
| 0.0302794498739221 | 4583 | MUC2 |
| 0.0299891166619757 | 80318 | GKAP1 |
| 0.0294623243414128 | 5918 | RARRES1 |
| 0.0279092225087342 | 162461 | TMEM92 |
| 0.0271509173124491 | 80352 | RNF39 |
| 0.0270873379258684 | 10500 | SEMA6C |
| 0.0270440552992568 | 145407 | C14orf37 |
| 0.0261513909384969 | 79887 | PLBD1 |
| 0.025867818407851 | 1969 | EPHA2 |
| 0.0249543206888824 | 3628 | INPP1 |
| 0.0246203667327912 | 1265 | CNN2 |
| 0.0244910080680791 | 3747 | KCNC2 |
| 0.024353339447048 | 192683 | SCAMP5 |
| 0.0241704060312738 | 79838 | TMC5 |
| 0.0240797728959675 | 843 | CASP10 |
| 0.0239382623015852 | 2901 | GRIK5 |
| 0.0236470070485605 | 65055 | REEP1 |
| 0.023558165555813 | 91149 | LOC91149 |
| 0.0229635378937681 | 54734 | RAB39 |
| 0.0225941846722822 | 7134 | TNNC1 |
| 0.021927963531071 | 460 | ASTN1 |
| 0.0214713293690434 | 84789 | MGC2889 |
| 0.0206013413633592 | 79632 | FAM184A |
| 0.0203016991673799 | 342527 | SMTNL2 |
| 0.0202530483810737 | 90019 | SYT8 |
| 0.0193506449538991 | 57026 | PDXP |
| 0.0191368142682077 | 83639 | TEX101 |
| 0.0171908960217093 | 120103 | SLC36A4 |
| 0.0166470619905928 | 5271 | SERPINB8 |
| 0.0156228945998449 | 55024 | BANK1 |
| 0.0149092906354022 | 54739 | XAF1 |
| 0.0144960308001917 | 11035 | RIPK3 |
| 0.013625705032631 | 9515 | STXBP5L |
| 0.013187602390103 | 29902 | C12orf24 |
| 0.0121749783871321 | 11013 | TMSB15A |
| 0.0117056733346026 | 4953 | ODC1 |
| 0.0112960196594732 | 80326 | WNT10A |
| 0.00957126385091118 | 131096 | KCNH8 |
| 0.00945021897072048 | 80023 | NRSN2 |
| 0.00931124777337205 | 26271 | FBXO5 |
| 0.00900565283703979 | 26154 | ABCA12 |
| 0.0087164388821683 | 10076 | PTPRU |
| 0.00790051653520074 | 26509 | MYOF |
| 0.00762762065259873 | 23624 | CBLC |
| 0.00756824135580562 | 55001 | TTC22 |
| 0.00682473788463022 | 116966 | WDR17 |
| 0.00610138526802581 | 27071 | DAPP1 |
| 0.00535654478487434 | 256472 | TMEM151A |
| 0.00522586565996572 | 57332 | CBX8 |
| 0.00518879049198783 | 201176 | ARHGAP27 |
| 0.00509188477438417 | 51087 | YBX2 |
| 0.0047030291919001 | 9201 | DCLK1 |
| 0.00457991687987879 | 51555 | PEX5L |
| 0.00413020170150375 | 255057 | C19orf26 |
| 0.00381321894352029 | 647946 | LOC647946 |
| 0.00355214999571835 | 9066 | SYT7 |
| 0.00326265537235233 | 84953 | MICALCL |
| 0.00319857008830961 | 285489 | DOK7 |
| 0.00304445077080572 | 84808 | C1orf170 |
| 0.00297830516197555 | 5122 | PCSK1 |
| 0.00286554264656813 | 57111 | RAB25 |
| 0.00271637217214627 | 140883 | ZNF280B |
| 0.00247855932757147 | 6672 | SP100 |
| 0.0018278984920645 | 2064 | ERBB2 |
| 0.00161835758767749 | 84740 | LOC84740 |
| 0.00063911009966386 | 255488 | RNF144B |

In some instances, one or both of training controller 215 and feature selector 235 determines or learns preprocessing parameters and/or approaches. For example, a preprocessing can include filtering expression data based on features selected by feature selector 235 (e.g., to include expression data corresponding to each selected gene, to exclude expression data corresponding to each non-selected gene, and/or to identify a subset of a set of selected genes for which expression data is to be assessed). Other exemplary preprocessing can include normalizing or standardizing data.

A machine learning (ML) execution handler 240 can use the architecture and learned parameters to process non-training data and generate a result. For example, ML execution handler 240 may receive expression data that corresponds to genes and to a subject not represented in the training expression data set. The expression data may (but need not) be preprocessed in accordance with a learned or identified preprocessing technique. The (preprocessed or original) expression data may be fed into a machine-learning model having an architecture (e.g., gradient boosting machine architecture 220 or regression architecture 225) used (or identified) during training and configured with learned parameters.

In some instances, a categorizer 245 identifies a category for the expression data set based on the execution of the machine-learning model. The execution may itself produce a result that includes the label, or the execution may include results that categorizer 245 can use to determine a category. For example, a result may include a probability that the expression data corresponds to a given category and/or a confidence of the probability. Categorizer 245 may then apply rules and/or transformations to map the probability and/or confidence to a category. In some instances, possible categories include a “neurally related” label, a “non-neurally related” category and an “unknown” category. As an illustration, a first category may be assigned if a result includes a probability greater than 50% that a tumor corresponds to a given class, and a second category may be otherwise assigned.

A treatment-candidate identifier 250 may use the category to identify one or more recommended treatments and/or one or more unrecommended treatments. For example, a result may include a degree to which a binary indication as to whether a checkpoint blockade therapy is predicted to be suitable for a given subject as a treatment candidate for a first-line treatment based on the category. For example, a checkpoint blockade therapy may be identified as a treatment candidate or candidate for a first-line treatment and/or sole treatment (e.g., indicating that it is not combined with another tumor-fighting treatment, such as chemotherapy or biotherapy) when a non-neurally related category is assigned. As another example, a treatment other than a checkpoint blockade therapy (e.g., chemotherapy, targeted therapy or biotherapy) can be identified as a treatment candidate or candidate for a first-line treatment when a neurally related category is assigned. As yet another (additional or alternative) example, a combination therapy that includes a checkpoint blockade therapy and another treatment can be identified as a treatment candidate or candidate for a first-line treatment when a neurally related category is assigned.

A panel specification controller 255 may use outputs from the machine-learning model and/or selected features (selected by feature selector 235) to identify specifications for a panel (e.g., a gene panel). The specifications may include an identifier of each of one, more or all genes to include in the panel. The specifications may include a list of genes amenable to be included in the panel (and for which expression data is informative of a category assignment). In some instances, panel specification controller 255 may identify each gene that is associated with a weight that is above a predefined absolute or relative threshold and/or a significance value that exceeds another predefined absolute or relative threshold (e.g., a p-value that is below another predefined threshold).

A communication interface 260 can collect results and communicate the result(s) (or a processed version thereof) to a user device or other system. For example, communication interface 260 may generate an output that identifies a subject, at least some of the expression data corresponding to the subject, an assigned category and an identified treatment candidate. The output may then presented and/or transmitted, which may facilitate a display of the output data, for example on a display of a computing device. As another example, communication interface 260 may generate an output that includes a list of genes for potential inclusion in a panel (potentially with weights and/or significance values associated with the genes), and the output may be displayed at a user device to facilitate design of a gene panel.

In some instances, expression levels in a subject of one or more, two or more, three or more, five or more, ten or more, twenty or more or fifty or more of the genes listed in Table 2 are analyzed. It will be appreciated that each or some of: one or more, two or more, three or more, five or more, ten or more, twenty or more or fifty or more of the genes listed in Table 2 can enhance activity of immune cells. In some instances, expression levels in a subject of one or more, two or more, three or more, five or more, ten or more, twenty or more or fifty or more of the genes listed in Table 3 are analyzed. In some instances, expression levels in a subject of one or more, two or more, three or more, five or more, ten or more, twenty or more or fifty or more of the genes listed in Table 4 are analyzed. The analysis can include generating a result that predicts whether one or more tumors of the subject are non-neurally related (versus neurally related), whether a disease (e.g., cancer) would respond to a treatment (e.g., as evidenced by slowed or stopped progression and/or survival for a period of time) that enhances activity of immune cells in the subject, and/or whether one or more tumors of the subject would respond (e.g., shrink in count, shrink in cumulative size, shrink in median tumor size, or shrink in average tumor size) to a treatment that enhances activity of immune cells in the subject, whether a disease (e.g., cancer) of the subject would respond to an immune checkpoint blockade treatment (e.g., as evidenced by slowed or stopped progression and/or survival for a period of time), and/or whether one or more tumors of the subject would respond (e.g., shrink in count, shrink in cumulative size, shrink in median tumor size, or shrink in average tumor size) to a checkpoint blockade therapy treatment.

TABLE 2

| Entrez Gene ID | Gene Symbol |
|---|---|
| 9900 | SV2A |
| 4684 | NCAM1 |
| 3694 | ITGB6 |
| 10045 | SH2D3A |
| 4070 | TACSTD2 |
| 64073 | C19orf33 |
| 2810 | SFN |
| 8153 | RND2 |
| 23612 | PHLDA3 |
| 5015 | OTX2 |
| 55357 | TBC1D2 |
| 79669 | C3orf52 |
| 311 | ANXA11 |
| 4440 | MSI1 |
| 80312 | TET1 |
| 84941 | HSH2D |
| 647024 | C6orf132 |
| 283248 | RCOR2 |
| 8837 | CFLAR |
| 3566 | IL4R |
| 729956 | SHISA7 |
| 113878 | DTX2 |
| 81622 | UNC93B1 |
| 2317 | FLNB |
| 22844 | FRMPD1 |
| 387104 | C6orf174 |
| 55964 | 3-Sep |
| 79570 | NKAIN1 |
| 199731 | CADM4 |
| 51560 | RAB6B |
| 55028 | C17orf80 |
| 3383 | ICAM1 |
| 547 | KIF1A |
| 57501 | KIAA1257 |

TABLE 3

| Entrez Gene ID | Gene Symbol |
|---|---|
| 9900 | SV2A |
| 4684 | NCAM1 |
| 3694 | ITGB6 |
| 10045 | SH2D3A |
| 4070 | TACSTD2 |
| 64073 | C19orf33 |
| 2810 | SFN |
| 8153 | RND2 |
| 23612 | PHLDA3 |
| 5015 | OTX2 |
| 55357 | TBC1D2 |
| 79669 | C3orf52 |
| 311 | ANXA11 |
| 4440 | MSI1 |
| 80312 | TET1 |
| 84941 | HSH2D |
| 647024 | C6orf132 |
| 283248 | RCOR2 |
| 8837 | CFLAR |
| 3566 | IL4R |

TABLE 4

| Entrez Gene ID | Gene Symbol |
|---|---|
| 9900 | SV2A |
| 4684 | NCAM1 |
| 3694 | ITGB6 |
| 10045 | SH2D3A |
| 4070 | TACSTD2 |
| 64073 | C19orf33 |
| 2810 | SFN |
| 8153 | RND2 |
| 23612 | PHLDA3 |
| 5015 | OTX2 |
| 55357 | TBC1D2 |

### IV. Example Model Training and Characterization

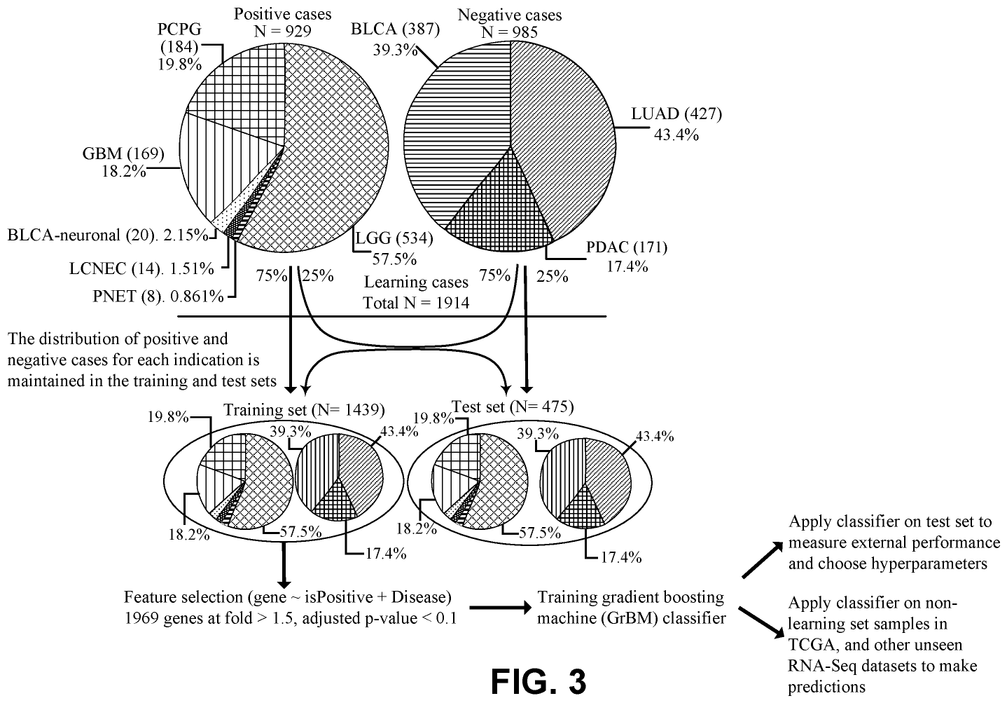

FIG. 3 shows an exemplary mappings for data labeling and uses thereof. In some instances, some or all of the depicted label mappings correspond to mappings identified by label mapper 205 and/or used (e.g., by training controller) to train a machine-learning model. In the depicted instance, a first set of tumor types are mapped to a neurally related label (“positive cases”), and a second set of tumor types are mapped to a non-neurally related category (“negative cases”). The first set includes brain tumors (glioblastoma (GBM) and low-grade glioma (LGG)), neuroendocrine tumors (pheochromocytoma—paraganglioma (PCPG), pancreatic neuroendocrine tumors (PNET) and lung adenocarcinoma—large cell neuroendocrine (LCNEC)) and other neurally related tumors (muscle-invasive bladder cancer —expression based neuronal subtype (BLCA-neuronal)). The second set may be defined so as to lack any brain or neuroendocrine tumors. For example, with respect to each of lung adenocarcinomas and muscle-invasive bladder cancer, tumors may be neuroendocrine tumors or may be non-neuroendocrine tumors. Thus, whether data from a particular subject having a lung adenocarcinomas and muscle-invasive bladder cancer is assigned to the first set versus the second set can depend on whether it is of a neuroendocrine type. In the illustrated instance, the second set includes pancreatic ductal adenocarcinoma (PDAC), non-neuroendocrine and non-brain lung adenocarinoma tumors (LUAD) and non-neuroendocrine and non-brain muscle-invasive bladder cancer (BLAC). Determining whether a tumor is of a neuroendocrine type can include applying a technique disclosed in (for example) Robertson A G et al., “Comprehensive molecular characterization of muscle-invasive bladder cancer”. Cell 17(3), 546-566 (October 2017) or Chen F et al., “Multiplatform-based molecular subtypes of non-small cell lung cancer” Oncogene 36, 1384-1393 (March 2017), each of which is hereby incorporated by reference in its entirety for all purposes.

The depicted illustration is representative of how data from a repository (e.g., The Cancer Genome Atlas) can be used to train a machine-learning model. In this example, each of 929 data elements corresponds to one of the listed types of tumors associated with the neurally related class, and each of 985 data elements corresponds to one of the listed types of tumors associated with the non-neurally related class. Each data element can include expression data for each of a plurality of genes. The data elements can be divided into a training set and a test set (e.g., such that a distribution of the data elements across the classes is approximately equal for the training set and the test set).

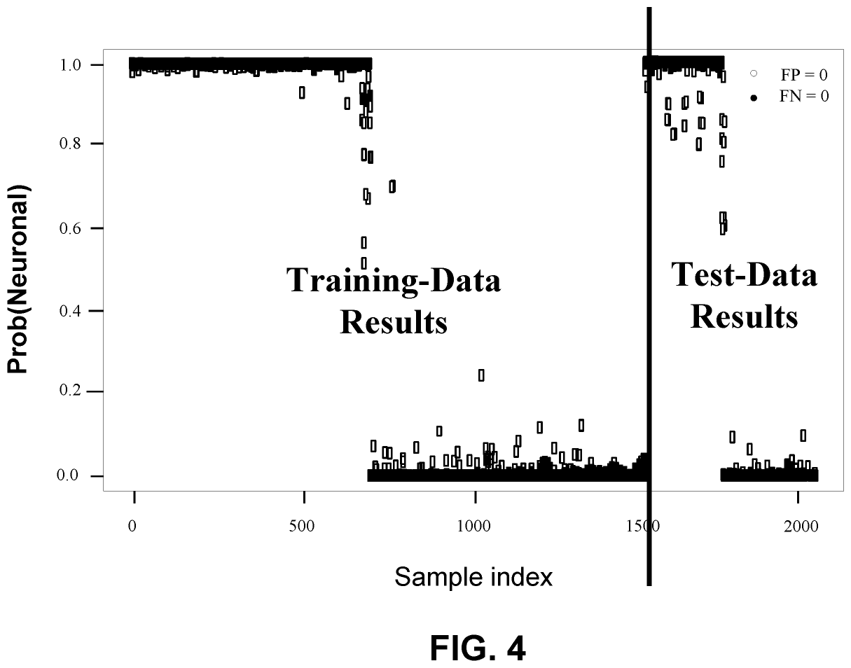

FIG. 4 shows training-data and test-data results generated using a trained machine-learning model. Specifically, the results correspond to data elements from The Cancer Genome Atlas that are divided into classes and test and training data sets as described with respect to FIG. 3. Feature selection was performed to remove data corresponding to genes having expression levels below a threshold in both classes. Of the remaining genes, a “discriminant” set of genes were identified as those having at least an above-threshold difference between the classes and also having an above-threshold significance. More specifically, in order for a gene to be characterized as a discriminant gene, its expression was required to be at least 1.5-fold different between the two classes. The difference was further required to be associated with an adjusted p-value of less than 0.1 in limma, when the limma model controls for disease indication. The adjusted p-value was calculated using the treat method, which used empirical Bayes moderated t-statistics with a minimum log-FC requirement. The discriminant set included 1969 genes.

With respect to the data depicted in FIG. 4, the example machine-learning model is configured to output a probability that the data corresponds to a neurally related tumor. A neurally related category is assigned if the probability exceeds 50% and a non-neurally related category is assigned otherwise. Instances in which the categories correctly corresponded to the actual class (as determined based on the mappings shown in FIG. 3) are represented by black rectangles. Instances in which a category was identified as neurally related, though the actual class was non-neurally related (false positive) are represented by filled circles. Instances in which a category was identified as non-neurally related, though the actual class was neurally related (false negative) are represented by open circles. As shown, there were no false negatives, and there were no false negatives. Thus, the machine-learning model was able to accurately learn to distinguish between these two classes of tumor.

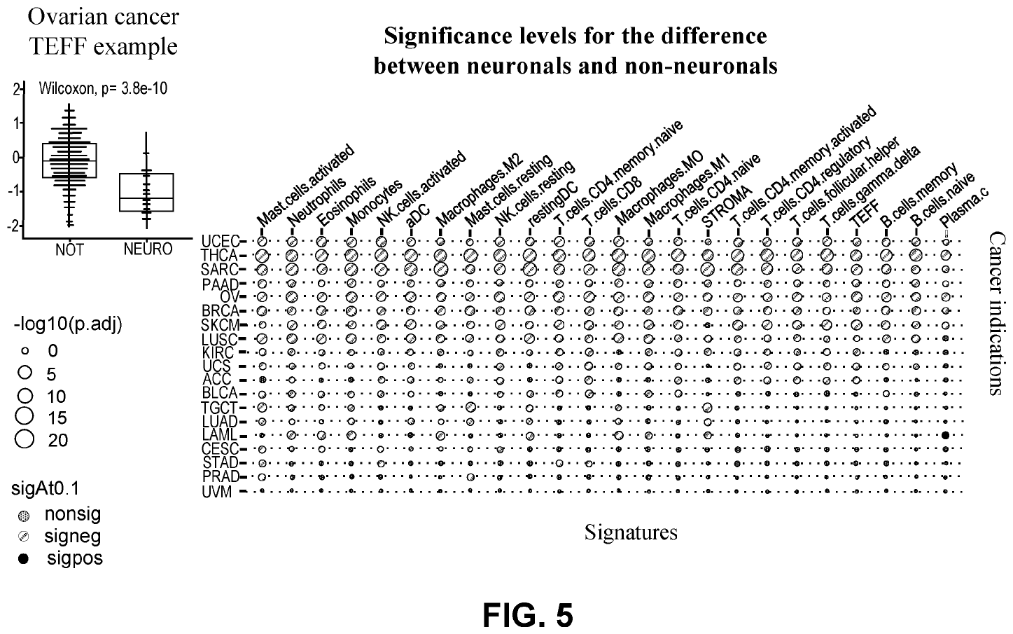

FIG. 5 illustrates a degree to which, for different tumor categories (rows), subsets corresponding to different ML-generated categories differ with respect to identified immune and stromal-infiltration signatures (columns). Each column in the dot-matrix represents a measure of immune response or stromal infiltration. Each row represents a tumor type. Each dot's size is scaled based on a significance level corresponding to differentiating tumors associated with a neurally related class (based on outputs of a machine-learning model trained and configured as described with respect to FIG. 4) and a non-neurally related class.

More specifically, with respect to each tumor type, a data set was collected that represented a set of tumors. Each data element in the set (corresponding to a single tumor) included gene-expression data. For each data element, the machine-learning model was used to classify the tumor as being neurally related or non-neurally related. With respect to each tumor, immune-response and stromal-infiltration metrics were also accessed. With respect to each tumor type and each immune-response or stromal-infiltration metric, a significance value was calculated that represented a significance of a difference of the metric across the two classes. The dot size correlates with the significance metric. The results indicate that, for some tumors, there are consistent and substantial differences across many immune-response and stromal-infiltration metrics between neurally related tumors and non-neurally related tumors. For other tumors, these differences are less pronounced. Potentially, for the other tumors one or more other tumor attributes dominate influence of these metrics, such that any difference caused by the neurally related/non-neurally related categorization is of reduced influence.

In some embodiments, an output from a machine-learning model, a category and/or a class can be used to identify a treatment approach and/or can be predictive of an efficacy of a treatment. For example, a neurally-related class designation may indicate that it is unlikely that checkpoint blockade therapy would be effective at treating a corresponding tumor (e.g., generally and/or without a prior conditioning treatment or a prior first-line treatment).

### V. Example Model Results

### V.A. Example 1

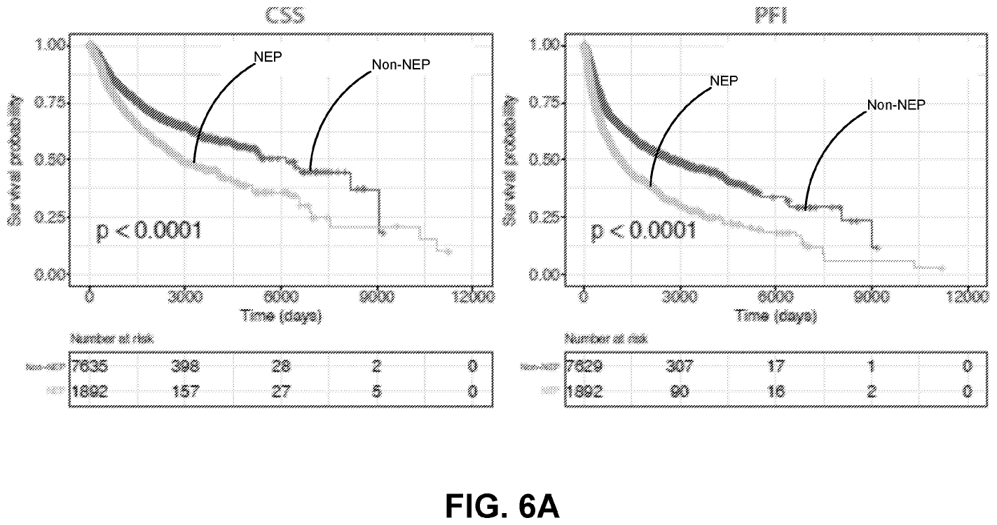

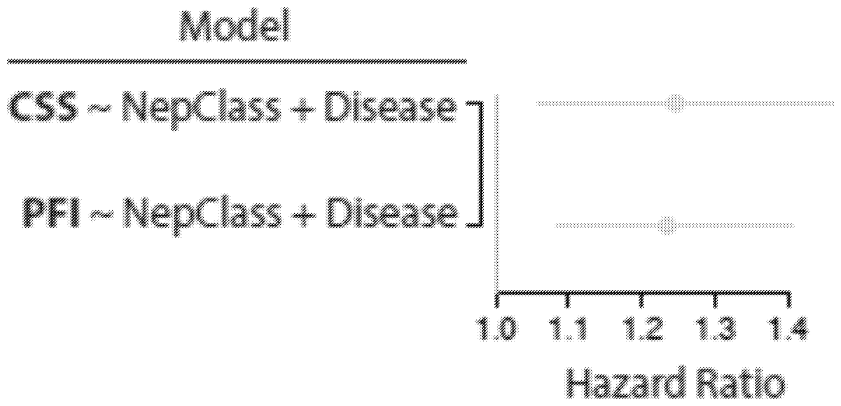

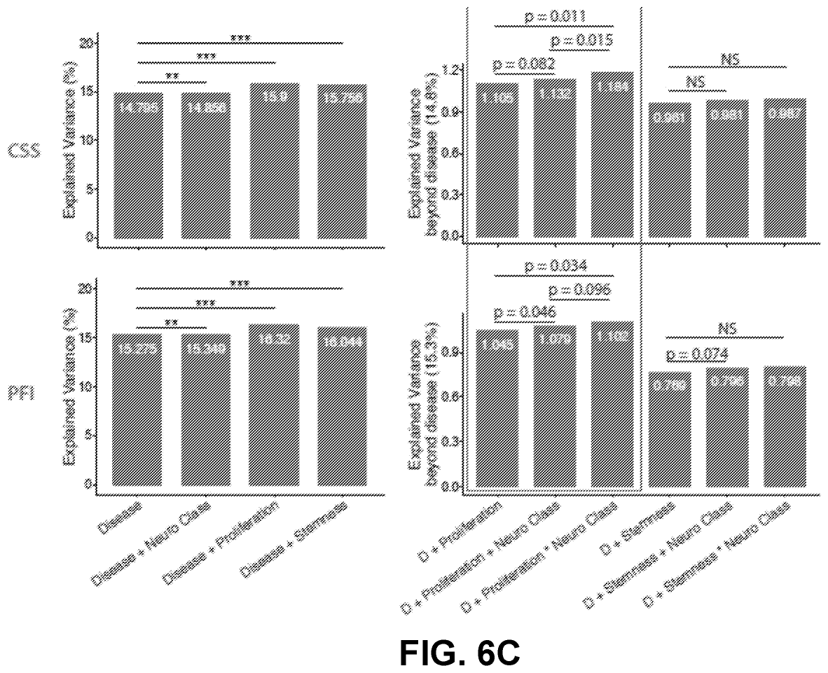

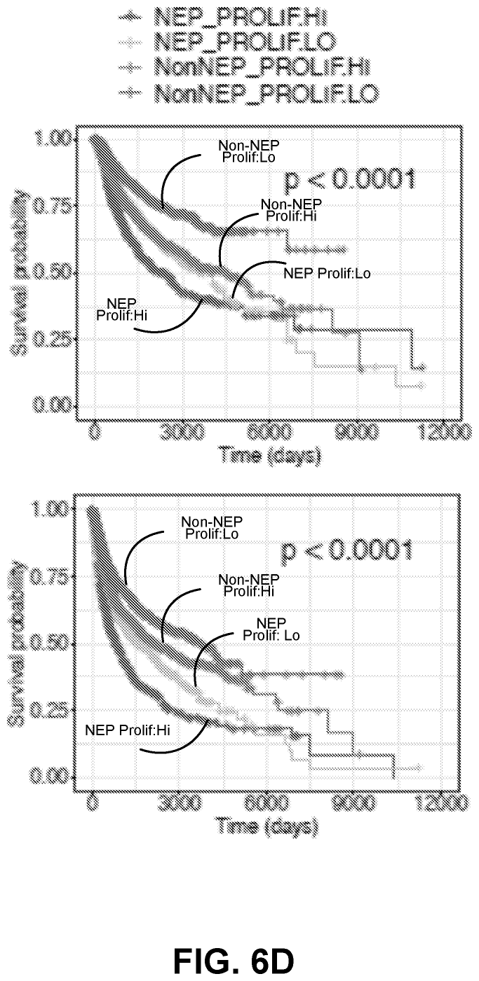

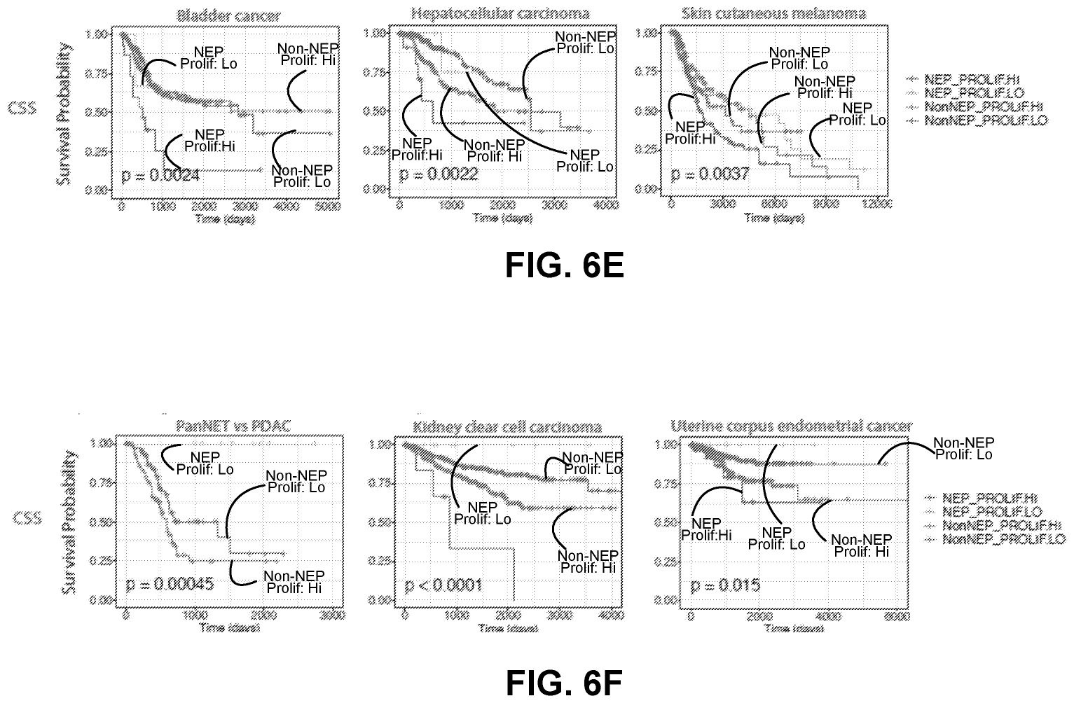

FIGS. 6A-6D show clinical data from treatment-naive samples from the Cancer Genome Atlas, separated by categories generated by a trained machine-learning model. Data in the Cancer Genome Atlas represents biospecimens from multiple hospitals (e.g., 5 or more) assumed to be providing standard-of-care treatment. More specifically, a machine-learning model, more fully discussed in Section V.E. below and referred to as NEPTUNE, was built based on a gradient-boosting-machine architecture was trained as described above with respect to FIG. 4. A separate test dataset including additional elements was then processed by the trained machine-learning model. The additional elements of the test dataset included expression data (determined using RNA-Seq) for each of a set of genes. The tumors evaluated in this Example were treatment-naive, so predictions were not confounded with different treatments (as lineage plasticity and neuroendocrine transformation are generally not observed across treatment-naive tumors, whereas such lineage plasticity and/or neuroendocrine transformation can occur in response to developed resistance to a treatment or as a result of a relapse). An output of the machine-learning model included a probability that the data element corresponded to a neurally related class. If the probability exceeded 50%, the data element was assigned to the neurally related class. Otherwise, it was assigned to a non-neurally related class.

Each data element corresponded to a subject, and outcome data of each subject was further tracked. Thus, survival and progression-free survival metrics could further be calculated. More specifically, time-series metrics were generated that identified, for a set of time points (relative to an initial pathologic diagnosis) and for each class (thicker line: neurally related class; thinner line: non-neurally related class), a percentage of the subjects corresponding to the class remained alive (left graph) and further a percentage of the subjects that remained alive and for which the tumor/cancer had not progressed (right graph). While tumor specimens were treatment-naive, subjects subsequently receive standard of care treatment (e.g., surgery or non-surgical treatments).

TCGA. Neurally related tumors in TCGA were observed to correspond with significantly poor cancer-specific survival (CSS) and progression-free interval (PFI) compared to non-neurally related tumors (FIG. 6A). To address the question whether individual cancer types may be driving this association, cancer type was controlled for in Cox proportional hazards regression models. Classification of neurally-related remained a significant risk factor for CSS and PFI (FIG. 6B). Due to the existence of two variants having a neural signature (1-low proliferating, well differentiated; 2-high proliferating, poorly differentiated), it was next investigated whether the neural programming phenotype had different survival associations based on levels of proliferation and sternness, i.e. whether the interaction term between the neurally related class and one of proliferation or sternness was significant. The explanatory power of a model that only included Disease (cancer type) was significantly increased by adding any one of neurally related category, proliferation and sternness (FIG. 6C, left panel). Proliferation was the most significant variable among the three, but the model had even greater power when proliferation was allowed to have different effect sizes for different neurally related categories. (For both CSS and PFI, proliferation had greater hazard ratio for neurally related tumors compared to non-neurally related tumors; suggesting proliferative tumors may be more aggressive in a neurally programmed state) (FIG. 6C, right panel). As indicated in Kaplan-Meier plots, subjects with high-proliferating neurally related tumors had the poorest outcome whereas those with low-proliferating non-neurally related tumors had the best clinical outcome (FIG. 6D). The aggressiveness of high-proliferating neurally related tumors was confirmed in multiple individual indications (e.g. melanoma, bladder and liver cancer) (FIG. 6E). Interestingly, low-proliferating neurally related tumors were indolent in some indications (FIG. 6F).

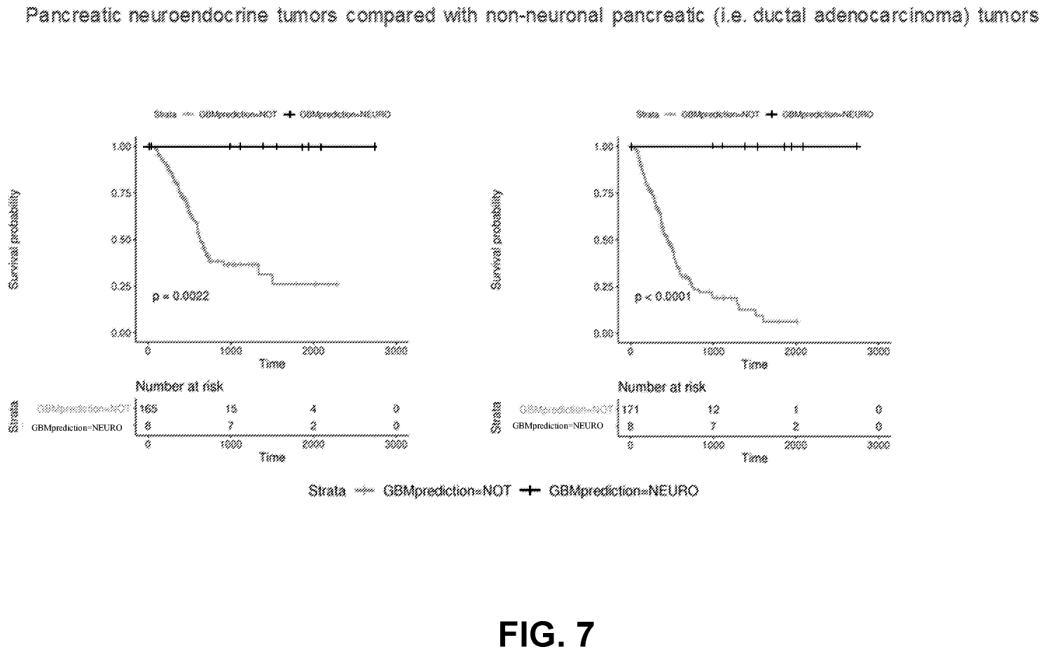

FIG. 7 shows the similar data but for pancreatic tumors. More specifically, the neurally related class corresponded to pancreatic neuroendocrine tumors, while the non-neurally related tumors corresponded to pancreatic ductal adenocarcinioma tumors. In this instance, survival metrics for the neurally related class exceeded those for the non-neurally related class. This data illustrates that low-proliferating neurally related tumors can be indolent.

### V.B. Example 2

Data sets were collected and analyzed as described in Example 1 and using the classifier described in Example 1, except that the data was further sub-divided based on a speed of proliferation (in addition to whether genetic-expression data for a given sample was assigned to a neurally related class or a non-neurally related class). Survival modeling was then performed to determine whether the neurally relating phenotype provided any additional informative as to survival data points beyond that provided based on the proliferation speed. To determine speed of proliferation, gene-expression data was processed to identify an estimated proliferation speed using the Hallmark G2M checkpoint gene set from MSigDB (as characterized at https://www.gsea-msigdb.org/gsea/msigdb/collections.jsp). Specifically, gene-expression data (RSEM values) were first log-transformed, and then standardized across samples for each gene in the proliferation signature. Standardized values (i.e., z-scores) were then averaged across genes to arrive at a proliferation score for each sample. For each of the high and low proliferation speed classifications, a median value was calculated across samples.

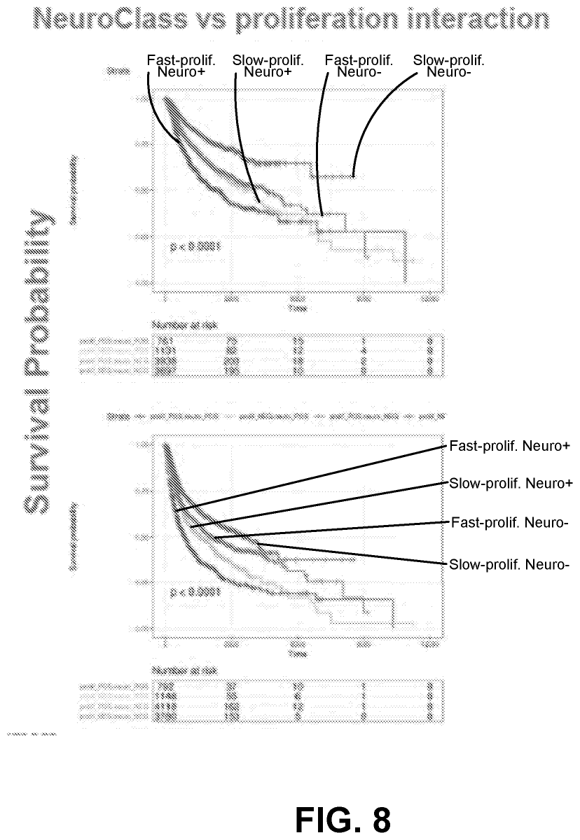

FIG. 8 shows Kaplan-Meier curves for cancer-specific survival (top) and progression-free survival (bottom). Subject outcomes were separated into four groups differentiated based on whether the gene-expression data was assigned to a neurally related class (versus non-neurally related class) and based on whether the gene-expression data was assigned to a high-proliferation class (versus a low-proliferation class). As illustrated in FIG. 8, accuracy varied across all four cohorts, and each of the two classifications (neurally v. non-neurally related and high v. low proliferation) appeared to influence prediction survival. The cohort associated with neurally related and high-proliferation classifications were associated with the lowest survival prospects, and the cohort associated with the non-neurally related and low-proliferation classifications were associated with the highest survival prospects. Notably, cohorts associated with (1) neurally related and high-proliferation classifications; and (2)non-neurally related and low-proliferation classifications were between the two extreme cohorts. Thus, it appears as though both the proliferation and neurally related classifications are informative as to survival prospects.

The survival-prospect distinction between the cohorts illustrates a difference in prognosis and disease activity between the cohorts, which may indicate a differential in treatment efficacy and/or suitability between subjects with a predicted neurally related classification (versus a non-neurally related classification) and/or on a prediction of proliferation speed. These results are consistent with understandings that pre-existing levels of intra-tumoral CD8 T cells (i.e., existing before therapy) are predictive of response to immune checkpoint blockade therapy. Since neurally related tumors are low in CD8 T-cell levels, these tumors are not likely to respond to immune checkpoint blockade therapy. The results indicate that, for proliferative neurally related tumors, a combination of chemotherapy and immune checkpoint blockade therapy may be effective at treating the tumors, while immune checkpoint blockade therapy alone or without chemotherapy may be a less effective treatment strategy for these tumors.

### V.C. Example 3

Genetic expression data derived from human breast cancer specimens (as described in Rueda et al., “Dynamics of Breast-Cancer Relapse Reveal Late-Curring ER-Positive Genomic Subgroups” Nature. 2019 March; 567(7748):399-404. doi: 10.1038/s41586-019-1007-8. Epub 2019 Mar. 13.) were collected from the METABRIC database. The Gene Ontology is as described in The Gene Ontology Consortium, “The Gene Ontology Resource: 20 years and still Going strong” Nucleic Acids Res. 2019 Jan. 8; 47(Database issue): D330-D338. The Gene Ontology (GO) neuron signature (also referred to below as ‘GO neuron’) (which lists genes that the GO identified as relating to neurons) was used to assign each specimen to a NEURO class: neurally related (NEP) or non-neurally related. More specifically, normalized gene expression data (microarray values) were standardized across samples for each gene in the GO Neuron signature, and standardized values (i.e., z-scores) were then averaged across genes to arrive at a neuronal score for each sample. Each specimen was further classified as being stem-like or well-differentiated (STEMNESS class) using the genetic expression data and the stemness signature from Miranda et al, “Cancer stemness, intratumoral heterogeneity, and immune response across cancers” Proc Natl Acad Sci USA. 2019 Apr. 30; 116(18):9020-9029. More specifically, a stemness characterization was further performed by standardizing across samples for each gene in the stemness signature, and standardized values (i.e., z-scores) were then averaged across genes to arrive at a stemness score for each sample. Table 5 identifies genes associated with the NEURO class and genes associated with the STEMNESS class. (Genes from the Hallmark G2M checkpoint gene set used to estimate proliferation speed are represented at rows 372-571 of Table 5. Genes associated with the sternness signature from Miranda et al., are represented at rows 263-371 of Table 5.)

TABLE 5

| ROW# | COLLECTION | SIGNATURE | GENE SYMBOL | ENTREZ ID |
|---|---|---|---|---|
| 1 | NEURON | GO Neuron | ACTL6B | 51412 |
| 2 | NEURON | GO Neuron | ADD2 | 119 |
| 3 | NEURON | GO Neuron | ATAT1 | 79969 |
| 4 | NEURON | GO Neuron | BAIAP2 | 10458 |
| 5 | NEURON | GO Neuron | BSN | 8927 |
| 6 | NEURON | GO Neuron | CA2 | 760 |
| 7 | NEURON | GO Neuron | CALB2 | 794 |
| 8 | NEURON | GO Neuron | CAMK2B | 816 |
| 9 | NEURON | GO Neuron | CAMK2G | 818 |
| 10 | NEURON | GO Neuron | CLIP2 | 7461 |
| 11 | NEURON | GO Neuron | CNIH2 | 254263 |
| 12 | NEURON | GO Neuron | DNER | 92737 |
| 13 | NEURON | GO Neuron | EPHA4 | 2043 |
| 14 | NEURON | GO Neuron | HTT | 3064 |
| 15 | NEURON | GO Neuron | LYNX1 | 66004 |
| 16 | NEURON | GO Neuron | MAPK8IP3 | 23162 |
| 17 | NEURON | GO Neuron | NEFM | 4741 |
| 18 | NEURON | GO Neuron | NFASC | 23114 |
| 19 | NEURON | GO Neuron | NRGN | 4900 |
| 20 | NEURON | GO Neuron | PIP5K1C | 23396 |
| 21 | NEURON | GO Neuron | PREX1 | 57580 |
| 22 | NEURON | GO Neuron | PTBP2 | 58155 |
| 23 | NEURON | GO Neuron | QDPR | 5860 |
| 24 | NEURON | GO Neuron | RAB3A | 5864 |
| 25 | NEURON | GO Neuron | SCAMP1 | 9522 |
| 26 | NEURON | GO Neuron | SDC3 | 9672 |
| 27 | NEURON | GO Neuron | SEZ6 | 124925 |
| 28 | NEURON | GO Neuron | SLC6A1 | 6529 |
| 29 | NEURON | GO Neuron | SNPH | 9751 |
| 30 | NEURON | GO Neuron | STX1A | 6804 |
| 31 | NEURON | GO Neuron | SV2A | 9900 |
| 32 | NEURON | GO Neuron | SYN1 | 6853 |
| 33 | NEURON | GO Neuron | SYNGR1 | 9145 |
| 34 | NEURON | GO Neuron | SYP | 6855 |
| 35 | NEURON | GO Neuron | SYT11 | 23208 |
| 36 | NEURON | GO Neuron | THY1 | 7070 |
| 37 | NEURON | GO Neuron | TMOD2 | 29767 |
| 38 | NEURON | GO Neuron | TPRG1L | 127262 |
| 39 | NEURON | GO Neuron | TUBB3 | 10381 |
| 40 | NEURON | GO Neuron | TWF2 | 11344 |
| 41 | NEURON | NEPC_Tsai2017 | AP3B2 | 8120 |
| 42 | NEURON | NEPC_Tsai2017 | APLP1 | 333 |
| 43 | NEURON | NEPC_Tsai2017 | ASCL1 | 429 |
| 44 | NEURON | NEPC_Tsai2017 | CCDC88A | 55704 |
| 45 | NEURON | NEPC_Tsai2017 | CDC25B | 994 |
| 46 | NEURON | NEPC_Tsai2017 | CRMP1 | 1400 |
| 47 | NEURON | NEPC_Tsai2017 | DLL3 | 10683 |
| 48 | NEURON | NEPC_Tsai2017 | DNMT1 | 1786 |
| 49 | NEURON | NEPC_Tsai2017 | ELAVL3 | 1995 |
| 50 | NEURON | NEPC_Tsai2017 | ELAVL4 | 1996 |
| 51 | NEURON | NEPC_Tsai2017 | ENO2 | 2026 |
| 52 | NEURON | NEPC_Tsai2017 | FAM161A | 84140 |
| 53 | NEURON | NEPC_Tsai2017 | FANCL | 55120 |
| 54 | NEURON | NEPC_Tsai2017 | FGF9 | 2254 |
| 55 | NEURON | NEPC_Tsai2017 | IGFBPL1 | 347252 |
| 56 | NEURON | NEPC_Tsai2017 | INA | 9118 |
| 57 | NEURON | NEPC_Tsai2017 | INSMI | 3642 |
| 58 | NEURON | NEPC_Tsai2017 | KCNC1 | 3746 |
| 59 | NEURON | NEPC_Tsai2017 | MIAT | 440823 |
| 60 | NEURON | NEPC_Tsai2017 | NKX2-1 | 7080 |
| 61 | NEURON | NEPC_Tsai2017 | NPPA | 4878 |
| 62 | NEURON | NEPC_Tsai2017 | NPTX1 | 4884 |
| 63 | NEURON | NEPC_Tsai2017 | PCSK1 | 5122 |
| 64 | NEURON | NEPC_Tsai2017 | PCSK2 | 5126 |
| 65 | NEURON | NEPC_Tsai2017 | PHF19 | 26147 |
| 66 | NEURON | NEPC_Tsai2017 | RNF183 | 138065 |
| 67 | NEURON | NEPC_Tsai2017 | RUNDC3A | 10900 |
| 68 | NEURON | NEPC_Tsai2017 | SEZ6 | 124925 |
| 69 | NEURON | NEPC_Tsai2017 | SH3GL2 | 6456 |
| 70 | NEURON | NEPC_Tsai2017 | SNAP25 | 6616 |
| 71 | NEURON | NEPC_Tsai2017 | SOX2 | 6657 |
| 72 | NEURON | NEPC_Tsai2017 | SRRM4 | 84530 |
| 73 | NEURON | NEPC_Tsai2017 | STMN1 | 3925 |
| 74 | NEURON | NEPC_Tsai2017 | TMEM145 | 284339 |
| 75 | NEURON | NEPC_Tsai2017 | TOX | 9760 |
| 76 | NEURON | NEPC_Tsai2017 | TUBB2B | 347733 |
| 77 | NEURON | NEPC_Tsai2017 | UNC13A | 23025 |
| 78 | NEURON | PanNE_Xu2016 | CARTPT | 9607 |
| 79 | NEURON | PanNE_Xu2016 | CHGA | 1113 |
| 80 | NEURON | PanNE_Xu2016 | CHGB | 1114 |
| 81 | NEURON | PanNE_Xu2016 | CHRNA3 | 1136 |
| 82 | NEURON | PanNE_Xu2016 | DBH | 1621 |
| 83 | NEURON | PanNE_Xu2016 | DLK1 | 8788 |
| 84 | NEURON | PanNE_Xu2016 | INSM1 | 3642 |
| 85 | NEURON | PanNE_Xu2016 | ISL1 | 3670 |
| 86 | NEURON | PanNE_Xu2016 | PHOX2B | 8929 |
| 87 | NEURON | Reactome Neuronal Sys | ACTN2 | 88 |
| 88 | NEURON | Reactome Neuronal Sys | ADCY1 | 107 |
| 89 | NEURON | Reactome Neuronal Sys | ADCY2 | 108 |
| 90 | NEURON | Reactome Neuronal Sys | ADCY5 | 111 |
| 91 | NEURON | Reactome Neuronal Sys | AKAP5 | 9495 |
| 92 | NEURON | Reactome Neuronal Sys | ALDH2 | 217 |
| 93 | NEURON | Reactome Neuronal Sys | ALDH5A1 | 7915 |
| 94 | NEURON | Reactome Neuronal Sys | AP2A1 | 160 |
| 95 | NEURON | Reactome Neuronal Sys | AP2A2 | 161 |
| 96 | NEURON | Reactome Neuronal Sys | AP2S1 | 1175 |
| 97 | NEURON | Reactome Neuronal Sys | APBA1 | 320 |
| 98 | NEURON | Reactome Neuronal Sys | APBA2 | 321 |
| 99 | NEURON | Reactome Neuronal Sys | ARHGEF9 | 23229 |
| 100 | NEURON | Reactome Neuronal Sys | CACNA1B | 774 |
| 101 | NEURON | Reactome Neuronal Sys | CACNA2D3 | 55799 |
| 102 | NEURON | Reactome Neuronal Sys | CACNB1 | 782 |
| 103 | NEURON | Reactome Neuronal Sys | CACNB4 | 785 |
| 104 | NEURON | Reactome Neuronal Sys | CACNG3 | 10368 |
| 105 | NEURON | Reactome Neuronal Sys | CACNG4 | 27092 |
| 106 | NEURON | Reactome Neuronal Sys | CACNG8 | 59283 |
| 107 | NEURON | Reactome Neuronal Sys | CAMK2A | 815 |
| 108 | NEURON | Reactome Neuronal Sys | CAMK2B | 816 |
| 109 | NEURON | Reactome Neuronal Sys | CAMK2D | 817 |
| 110 | NEURON | Reactome Neuronal Sys | CAMK2G | 818 |
| 111 | NEURON | Reactome Neuronal Sys | CAMKK1 | 84254 |
| 112 | NEURON | Reactome Neuronal Sys | CASK | 8573 |
| 113 | NEURON | Reactome Neuronal Sys | CHAT | 1103 |
| 114 | NEURON | Reactome Neuronal Sys | CHRNA1 | 1134 |
| 115 | NEURON | Reactome Neuronal Sys | CHRNA2 | 1135 |
| 116 | NEURON | Reactome Neuronal Sys | CHRNA3 | 1136 |
| 117 | NEURON | Reactome Neuronal Sys | CHRNA4 | 1137 |
| 118 | NEURON | Reactome Neuronal Sys | CHRNA5 | 1138 |
| 119 | NEURON | Reactome Neuronal Sys | CHRNA7 | 1139 |
| 120 | NEURON | Reactome Neuronal Sys | CHRNA9 | 55584 |
| 121 | NEURON | Reactome Neuronal Sys | CHRNB2 | 1141 |
| 122 | NEURON | Reactome Neuronal Sys | CHRNB3 | 1142 |
| 123 | NEURON | Reactome Neuronal Sys | CHRNB4 | 1143 |
| 124 | NEURON | Reactome Neuronal Sys | CHRND | 1144 |
| 125 | NEURON | Reactome Neuronal Sys | CHRNE | 1145 |
| 126 | NEURON | Reactome Neuronal Sys | CHRNG | 1146 |
| 127 | NEURON | Reactome Neuronal Sys | COMT | 1312 |
| 128 | NEURON | Reactome Neuronal Sys | CPLX1 | 10815 |
| 129 | NEURON | Reactome Neuronal Sys | DLG1 | 1739 |
| 130 | NEURON | Reactome Neuronal Sys | DLG2 | 1740 |
| 131 | NEURON | Reactome Neuronal Sys | DLG3 | 1741 |
| 132 | NEURON | Reactome Neuronal Sys | DLG4 | 1742 |
| 133 | NEURON | Reactome Neuronal Sys | DLGAP1 | 9229 |
| 134 | NEURON | Reactome Neuronal Sys | EPB41L1 | 2036 |
| 135 | NEURON | Reactome Neuronal Sys | EPB41L2 | 2037 |
| 136 | NEURON | Reactome Neuronal Sys | EPB41L3 | 23136 |
| 137 | NEURON | Reactome Neuronal Sys | GABBR1 | 2550 |
| 138 | NEURON | Reactome Neuronal Sys | GABBR2 | 9568 |
| 139 | NEURON | Reactome Neuronal Sys | GABRA1 | 2554 |
| 140 | NEURON | Reactome Neuronal Sys | GABRA2 | 2555 |
| 141 | NEURON | Reactome Neuronal Sys | GABRA3 | 2556 |
| 142 | NEURON | Reactome Neuronal Sys | GABRA4 | 2557 |
| 143 | NEURON | Reactome Neuronal Sys | GABRA5 | 2558 |
| 144 | NEURON | Reactome Neuronal Sys | GABRB2 | 2561 |
| 145 | NEURON | Reactome Neuronal Sys | GABRB3 | 2562 |
| 146 | NEURON | Reactome Neuronal Sys | GABRG2 | 2566 |
| 147 | NEURON | Reactome Neuronal Sys | GAD1 | 2571 |
| 148 | NEURON | Reactome Neuronal Sys | GAD2 | 2572 |
| 149 | NEURON | Reactome Neuronal Sys | GLS | 2744 |
| 150 | NEURON | Reactome Neuronal Sys | GNAI1 | 2770 |
| 151 | NEURON | Reactome Neuronal Sys | GNAI2 | 2771 |
| 152 | NEURON | Reactome Neuronal Sys | GNAT3 | 346562 |
| 153 | NEURON | Reactome Neuronal Sys | GNB4 | 59345 |
| 154 | NEURON | Reactome Neuronal Sys | GNG2 | 54331 |
| 155 | NEURON | Reactome Neuronal Sys | GNG3 | 2785 |
| 156 | NEURON | Reactome Neuronal Sys | GNG4 | 2786 |
| 157 | NEURON | Reactome Neuronal Sys | GNG7 | 2788 |
| 158 | NEURON | Reactome Neuronal Sys | GNGT1 | 2792 |
| 159 | NEURON | Reactome Neuronal Sys | GNGT2 | 2793 |
| 160 | NEURON | Reactome Neuronal Sys | GRIA1 | 2890 |
| 161 | NEURON | Reactome Neuronal Sys | GRIA2 | 2891 |
| 162 | NEURON | Reactome Neuronal Sys | GRIA3 | 2892 |
| 163 | NEURON | Reactome Neuronal Sys | GRIA4 | 2893 |
| 164 | NEURON | Reactome Neuronal Sys | GRIK3 | 2899 |
| 165 | NEURON | Reactome Neuronal Sys | GRIK5 | 2901 |
| 166 | NEURON | Reactome Neuronal Sys | GRIN1 | 2902 |
| 167 | NEURON | Reactome Neuronal Sys | GRIN2A | 2903 |
| 168 | NEURON | Reactome Neuronal Sys | HCN1 | 348980 |
| 169 | NEURON | Reactome Neuronal Sys | HCN2 | 610 |
| 170 | NEURON | Reactome Neuronal Sys | HOMER1 | 9456 |
| 171 | NEURON | Reactome Neuronal Sys | HRAS | 3265 |
| 172 | NEURON | Reactome Neuronal Sys | KCNA1 | 3736 |
| 173 | NEURON | Reactome Neuronal Sys | KCNA10 | 3744 |
| 174 | NEURON | Reactome Neuronal Sys | KCNA2 | 3737 |
| 175 | NEURON | Reactome Neuronal Sys | KCNA7 | 3743 |
| 176 | NEURON | Reactome Neuronal Sys | KCNAB2 | 8514 |
| 177 | NEURON | Reactome Neuronal Sys | KCNB1 | 3745 |
| 178 | NEURON | Reactome Neuronal Sys | KCNC2 | 3747 |
| 179 | NEURON | Reactome Neuronal Sys | KCND2 | 3751 |
| 180 | NEURON | Reactome Neuronal Sys | KCND3 | 3752 |
| 181 | NEURON | Reactome Neuronal Sys | KCNF1 | 3754 |
| 182 | NEURON | Reactome Neuronal Sys | KCNH3 | 23416 |
| 183 | NEURON | Reactome Neuronal Sys | KCNH6 | 81033 |
| 184 | NEURON | Reactome Neuronal Sys | KCNJ10 | 3766 |
| 185 | NEURON | Reactome Neuronal Sys | KCNJ15 | 3772 |
| 186 | NEURON | Reactome Neuronal Sys | KCNJ3 | 3760 |
| 187 | NEURON | Reactome Neuronal Sys | KCNJ4 | 3761 |
| 188 | NEURON | Reactome Neuronal Sys | KCNJ9 | 3765 |
| 189 | NEURON | Reactome Neuronal Sys | KCNK1 | 3775 |
| 190 | NEURON | Reactome Neuronal Sys | KCNK16 | 83795 |
| 191 | NEURON | Reactome Neuronal Sys | KCNK17 | 89822 |
| 192 | NEURON | Reactome Neuronal Sys | KCNK7 | 10089 |
| 193 | NEURON | Reactome Neuronal Sys | KCNMA1 | 3778 |
| 194 | NEURON | Reactome Neuronal Sys | KCNMB3 | 27094 |
| 195 | NEURON | Reactome Neuronal Sys | KCNMB4 | 27345 |
| 196 | NEURON | Reactome Neuronal Sys | KCNN3 | 3782 |
| 197 | NEURON | Reactome Neuronal Sys | KCNQ2 | 3785 |
| 198 | NEURON | Reactome Neuronal Sys | KCNQ3 | 3786 |
| 199 | NEURON | Reactome Neuronal Sys | KCNQ5 | 56479 |
| 200 | NEURON | Reactome Neuronal Sys | KCNV2 | 169522 |
| 201 | NEURON | Reactome Neuronal Sys | LIN7C | 55327 |
| 202 | NEURON | Reactome Neuronal Sys | LRRTM2 | 26045 |
| 203 | NEURON | Reactome Neuronal Sys | LRRTM3 | 347731 |
| 204 | NEURON | Reactome Neuronal Sys | MYO6 | 4646 |
| 205 | NEURON | Reactome Neuronal Sys | NCALD | 83988 |
| 206 | NEURON | Reactome Neuronal Sys | NEFL | 4747 |
| 207 | NEURON | Reactome Neuronal Sys | NLGN2 | 57555 |
| 208 | NEURON | Reactome Neuronal Sys | NLGN3 | 54413 |
| 209 | NEURON | Reactome Neuronal Sys | NLGN4X | 57502 |
| 210 | NEURON | Reactome Neuronal Sys | NRXN1 | 9378 |
| 211 | NEURON | Reactome Neuronal Sys | NRXN2 | 9379 |
| 212 | NEURON | Reactome Neuronal Sys | NRXN3 | 9369 |
| 213 | NEURON | Reactome Neuronal Sys | NSF | 4905 |
| 214 | NEURON | Reactome Neuronal Sys | PANX1 | 24145 |
| 215 | NEURON | Reactome Neuronal Sys | PDLIM5 | 10611 |
| 216 | NEURON | Reactome Neuronal Sys | PLCB1 | 23236 |
| 217 | NEURON | Reactome Neuronal Sys | PPFIA2 | 8499 |
| 218 | NEURON | Reactome Neuronal Sys | PPFIA3 | 8541 |
| 219 | NEURON | Reactome Neuronal Sys | PRKCA | 5578 |
| 220 | NEURON | Reactome Neuronal Sys | PRKCB | 5579 |
| 221 | NEURON | Reactome Neuronal Sys | PRKCG | 5582 |
| 222 | NEURON | Reactome Neuronal Sys | PTPRD | 5789 |
| 223 | NEURON | Reactome Neuronal Sys | PTPRS | 5802 |
| 224 | NEURON | Reactome Neuronal Sys | RAB3A | 5864 |
| 225 | NEURON | Reactome Neuronal Sys | RASGRF1 | 5923 |
| 226 | NEURON | Reactome Neuronal Sys | RASGRF2 | 5924 |
| 227 | NEURON | Reactome Neuronal Sys | RPS6KA2 | 6196 |
| 228 | NEURON | Reactome Neuronal Sys | RPS6KA3 | 6197 |
| 229 | NEURON | Reactome Neuronal Sys | SHANK1 | 50944 |
| 230 | NEURON | Reactome Neuronal Sys | SIPA1L1 | 26037 |
| 231 | NEURON | Reactome Neuronal Sys | SLC17A7 | 57030 |
| 232 | NEURON | Reactome Neuronal Sys | SLC18A2 | 6571 |
| 233 | NEURON | Reactome Neuronal Sys | SLC18A3 | 6572 |
| 234 | NEURON | Reactome Neuronal Sys | SLC1A1 | 6505 |
| 235 | NEURON | Reactome Neuronal Sys | SLC1A2 | 6506 |
| 236 | NEURON | Reactome Neuronal Sys | SLC1A3 | 6507 |
| 237 | NEURON | Reactome Neuronal Sys | SLC1A7 | 6512 |
| 238 | NEURON | Reactome Neuronal Sys | SLC22A1 | 6580 |
| 239 | NEURON | Reactome Neuronal Sys | SLC22A2 | 6582 |
| 240 | NEURON | Reactome Neuronal Sys | SLC32A1 | 140679 |
| 241 | NEURON | Reactome Neuronal Sys | SLC38A1 | 81539 |
| 242 | NEURON | Reactome Neuronal Sys | SLC5A7 | 60482 |
| 243 | NEURON | Reactome Neuronal Sys | SLC6A1 | 6529 |
| 244 | NEURON | Reactome Neuronal Sys | SLC6A11 | 6538 |
| 245 | NEURON | Reactome Neuronal Sys | SLC6A13 | 6540 |
| 246 | NEURON | Reactome Neuronal Sys | SLC6A4 | 6532 |
| 247 | NEURON | Reactome Neuronal Sys | SNAP25 | 6616 |
| 248 | NEURON | Reactome Neuronal Sys | STX1A | 6804 |
| 249 | NEURON | Reactome Neuronal Sys | STXBP1 | 6812 |
| 250 | NEURON | Reactome Neuronal Sys | SYN1 | 6853 |
| 251 | NEURON | Reactome Neuronal Sys | SYT1 | 6857 |
| 252 | NEURON | Reactome Neuronal Sys | SYT7 | 9066 |
| 253 | NEURON | Reactome Neuronal Sys | TSPAN7 | 7102 |
| 254 | NEURON | Reactome Neuronal Sys | UNC13B | 10497 |
| 255 | NEURON | Robertson Neuronal Diff | APLP1 | 333 |
| 256 | NEURON | Robertson Neuronal Diff | GNG4 | 2786 |
| 257 | NEURON | Robertson Neuronal Diff | MSI1 | 4440 |
| 258 | NEURON | Robertson Neuronal Diff | PEG10 | 23089 |
| 259 | NEURON | Robertson Neuronal Diff | PLEKHG4B | 153478 |
| 260 | NEURON | Robertson Neuronal Diff | RND2 | 8153 |
| 261 | NEURON | Robertson Neuronal Diff | SOX2 | 6657 |
| 262 | NEURON | Robertson Neuronal Diff | TUBB2B | 347733 |
| 263 | Stemness | Miranda et al PNAS 2019 | DNMT3B | 1789 |
| 264 | Stemness | Miranda et al PNAS 2019 | PFAS | 5198 |
| 265 | Stemness | Miranda et al PNAS 2019 | XRCC5 | 7520 |
| 266 | Stemness | Miranda et al PNAS 2019 | HAUS6 | 54801 |
| 267 | Stemness | Miranda et al PNAS 2019 | TET1 | 80312 |
| 268 | Stemness | Miranda et al PNAS 2019 | IGF2BP1 | 10642 |
| 269 | Stemness | Miranda et al PNAS 2019 | PLAA | 9373 |
| 270 | Stemness | Miranda et al PNAS 2019 | TEX10 | 54881 |
| 271 | Stemness | Miranda et al PNAS 2019 | MSH6 | 2956 |
| 272 | Stemness | Miranda et al PNAS 2019 | DLGAP5 | 9787 |
| 273 | Stemness | Miranda et al PNAS 2019 | SKIV2L2 | 23517 |
| 274 | Stemness | Miranda et al PNAS 2019 | SOHLH2 | 54937 |
| 275 | Stemness | Miranda et al PNAS 2019 | RRAS2 | 22800 |
| 276 | Stemness | Miranda et al PNAS 2019 | PAICS | 10606 |
| 277 | Stemness | Miranda et al PNAS 2019 | CPSF3 | 51692 |
| 278 | Stemness | Miranda et al PNAS 2019 | LIN28B | 389421 |
| 279 | Stemness | Miranda et al PNAS 2019 | IPO5 | 3843 |
| 280 | Stemness | Miranda et al PNAS 2019 | BMPR1A | 657 |
| 281 | Stemness | Miranda et al PNAS 2019 | ZNF788 | 388507 |
| 282 | Stemness | Miranda et al PNAS 2019 | ASCC3 | 10973 |
| 283 | Stemness | Miranda et al PNAS 2019 | FANCB | 2187 |
| 284 | Stemness | Miranda et al PNAS 2019 | HMGA2 | 8091 |
| 285 | Stemness | Miranda et al PNAS 2019 | TRIM24 | 8805 |
| 286 | Stemness | Miranda et al PNAS 2019 | ORC1 | 4998 |
| 287 | Stemness | Miranda et al PNAS 2019 | HDAC2 | 3066 |
| 288 | Stemness | Miranda et al PNAS 2019 | HESX1 | 8820 |
| 289 | Stemness | Miranda et al PNAS 2019 | INHBE | 83729 |
| 290 | Stemness | Miranda et al PNAS 2019 | MIS18A | 54069 |
| 291 | Stemness | Miranda et al PNAS 2019 | DCUN1D5 | 84259 |
| 292 | Stemness | Miranda et al PNAS 2019 | MRPL3 | 11222 |
| 293 | Stemness | Miranda et al PNAS 2019 | CENPH | 64946 |
| 294 | Stemness | Miranda et al PNAS 2019 | MYCN | 4613 |
| 295 | Stemness | Miranda et al PNAS 2019 | HAUS1 | 115106 |
| 296 | Stemness | Miranda et al PNAS 2019 | GDF3 | 9573 |
| 297 | Stemness | Miranda et al PNAS 2019 | TBCE | 6905 |
| 298 | Stemness | Miranda et al PNAS 2019 | RIOK2 | 55781 |
| 299 | Stemness | Miranda et al PNAS 2019 | BCKDHB | 594 |
| 300 | Stemness | Miranda et al PNAS 2019 | RAD1 | 5810 |
| 301 | Stemness | Miranda et al PNAS 2019 | NREP | 9315 |
| 302 | Stemness | Miranda et al PNAS 2019 | ADH5 | 128 |
| 303 | Stemness | Miranda et al PNAS 2019 | PLRG1 | 5356 |
| 304 | Stemness | Miranda et al PNAS 2019 | ROR1 | 4919 |
| 305 | Stemness | Miranda et al PNAS 2019 | RAB3B | 5865 |
| 306 | Stemness | Miranda et al PNAS 2019 | DIAPH3 | 81624 |
| 307 | Stemness | Miranda et al PNAS 2019 | GNL2 | 29889 |
| 308 | Stemness | Miranda et al PNAS 2019 | FGF2 | 2247 |
| 309 | Stemness | Miranda et al PNAS 2019 | NMNAT2 | 23057 |
| 310 | Stemness | Miranda et al PNAS 2019 | KIF20A | 10112 |
| 311 | Stemness | Miranda et al PNAS 2019 | CENPI | 2491 |
| 312 | Stemness | Miranda et al PNAS 2019 | DDX1 | 1653 |
| 313 | Stemness | Miranda et al PNAS 2019 | XXYLT1 | 152002 |
| 314 | Stemness | Miranda et al PNAS 2019 | GPR176 | 11245 |
| 315 | Stemness | Miranda et al PNAS 2019 | BBS9 | 27241 |
| 316 | Stemness | Miranda et al PNAS 2019 | C14orf166 | 51637 |
| 317 | Stemness | Miranda et al PNAS 2019 | BOD1 | 91272 |
| 318 | Stemness | Miranda et al PNAS 2019 | CDC123 | 8872 |
| 319 | Stemness | Miranda et al PNAS 2019 | SNRPD3 | 6634 |
| 320 | Stemness | Miranda et al PNAS 2019 | FAM118B | 79607 |
| 321 | Stemness | Miranda et al PNAS 2019 | DPH3 | 285381 |
| 322 | Stemness | Miranda et al PNAS 2019 | EIF2B3 | 8891 |
| 323 | Stemness | Miranda et al PNAS 2019 | RPF2 | 84154 |
| 324 | Stemness | Miranda et al PNAS 2019 | APLP1 | 333 |
| 325 | Stemness | Miranda et al PNAS 2019 | DACT1 | 51339 |
| 326 | Stemness | Miranda et al PNAS 2019 | PDHB | 5162 |
| 327 | Stemness | Miranda et al PNAS 2019 | C14orf119 | 55017 |
| 328 | Stemness | Miranda et al PNAS 2019 | DTD1 | 92675 |
| 329 | Stemness | Miranda et al PNAS 2019 | SAMM50 | 25813 |
| 330 | Stemness | Miranda et al PNAS 2019 | CCL26 | 10344 |
| 331 | Stemness | Miranda et al PNAS 2019 | MED20 | 9477 |
| 332 | Stemness | Miranda et al PNAS 2019 | UTP6 | 55813 |
| 333 | Stemness | Miranda et al PNAS 2019 | RARS2 | 57038 |
| 334 | Stemness | Miranda et al PNAS 2019 | ARMCX2 | 9823 |
| 335 | Stemness | Miranda et al PNAS 2019 | RARS | 5917 |
| 336 | Stemness | Miranda et al PNAS 2019 | MTHFD2 | 10797 |
| 337 | Stemness | Miranda et al PNAS 2019 | DHX15 | 1665 |
| 338 | Stemness | Miranda et al PNAS 2019 | HTR7 | 3363 |
| 339 | Stemness | Miranda et al PNAS 2019 | MTHFD1L | 25902 |
| 340 | Stemness | Miranda et al PNAS 2019 | ARMC9 | 80210 |
| 341 | Stemness | Miranda et al PNAS 2019 | XPOT | 11260 |
| 342 | Stemness | Miranda et al PNAS 2019 | IARS | 3376 |
| 343 | Stemness | Miranda et al PNAS 2019 | HDX | 139324 |
| 344 | Stemness | Miranda et al PNAS 2019 | ACTRT3 | 84517 |
| 345 | Stemness | Miranda et al PNAS 2019 | ERCC2 | 2068 |
| 346 | Stemness | Miranda et al PNAS 2019 | TBC1D16 | 125058 |
| 347 | Stemness | Miranda et al PNAS 2019 | GARS | 2617 |
| 348 | Stemness | Miranda et al PNAS 2019 | KIF7 | 374654 |
| 349 | Stemness | Miranda et al PNAS 2019 | UBE2K | 3093 |
| 350 | Stemness | Miranda et al PNAS 2019 | SLC25A3 | 5250 |
| 351 | Stemness | Miranda et al PNAS 2019 | ICMT | 23463 |
| 352 | Stemness | Miranda et al PNAS 2019 | UGGT2 | 55757 |
| 353 | Stemness | Miranda et al PNAS 2019 | ATP11C | 286410 |
| 354 | Stemness | Miranda et al PNAS 2019 | SLC24A1 | 9187 |
| 355 | Stemness | Miranda et al PNAS 2019 | EIF2AK4 | 440275 |
| 356 | Stemness | Miranda et al PNAS 2019 | GPX8 | 493869 |
| 357 | Stemness | Miranda et al PNAS 2019 | ALX1 | 8092 |
| 358 | Stemness | Miranda et al PNAS 2019 | OSTC | 58505 |
| 359 | Stemness | Miranda et al PNAS 2019 | TRPC4 | 7223 |
| 360 | Stemness | Miranda et al PNAS 2019 | HAS2 | 3037 |
| 361 | Stemness | Miranda et al PNAS 2019 | FZD2 | 2535 |
| 362 | Stemness | Miranda et al PNAS 2019 | TRNT1 | 51095 |
| 363 | Stemness | Miranda et al PNAS 2019 | MMADHC | 27249 |
| 364 | Stemness | Miranda et al PNAS 2019 | SNX8 | 29886 |
| 365 | Stemness | Miranda et al PNAS 2019 | CDH6 | 1004 |
| 366 | Stemness | Miranda et al PNAS 2019 | HAT1 | 8520 |
| 367 | Stemness | Miranda et al PNAS 2019 | SEC11A | 23478 |
| 368 | Stemness | Miranda et al PNAS 2019 | DIMT1 | 27292 |
| 369 | Stemness | Miranda et al PNAS 2019 | TM2D2 | 83877 |
| 370 | Stemness | Miranda et al PNAS 2019 | FST | 10468 |
| 371 | Stemness | Miranda et al PNAS 2019 | GBE1 | 2632 |
| 372 | HALLMARK | G2M_CHECKPOINT | ABL1 | 25 |
| 373 | HALLMARK | G2M_CHECKPOINT | AMD1 | 262 |
| 374 | HALLMARK | G2M_CHECKPOINT | ARID4A | 5926 |
| 375 | HALLMARK | G2M_CHECKPOINT | ATF5 | 22809 |
| 376 | HALLMARK | G2M_CHECKPOINT | ATRX | 546 |
| 377 | HALLMARK | G2M_CHECKPOINT | AURKA | 6790 |
| 378 | HALLMARK | G2M_CHECKPOINT | AURKB | 9212 |
| 379 | HALLMARK | G2M_CHECKPOINT | BARD1 | 580 |
| 380 | HALLMARK | G2M_CHECKPOINT | BCL3 | 602 |
| 381 | HALLMARK | G2M_CHECKPOINT | BIRC5 | 332 |
| 382 | HALLMARK | G2M_CHECKPOINT | BRCA2 | 675 |
| 383 | HALLMARK | G2M_CHECKPOINT | BUB1 | 699 |
| 384 | HALLMARK | G2M_CHECKPOINT | BUB3 | 9184 |
| 385 | HALLMARK | G2M_CHECKPOINT | CASC5 | 57082 |
| 386 | HALLMARK | G2M_CHECKPOINT | CASP8AP2 | 9994 |
| 387 | HALLMARK | G2M_CHECKPOINT | CBX1 | 10951 |
| 388 | HALLMARK | G2M_CHECKPOINT | CCNA2 | 890 |
| 389 | HALLMARK | G2M_CHECKPOINT | CCNB2 | 9133 |
| 390 | HALLMARK | G2M_CHECKPOINT | CCND1 | 595 |
| 391 | HALLMARK | G2M_CHECKPOINT | CCNF | 899 |
| 392 | HALLMARK | G2M_CHECKPOINT | CCNT1 | 904 |
| 393 | HALLMARK | G2M_CHECKPOINT | CDC20 | 991 |
| 394 | HALLMARK | G2M_CHECKPOINT | CDC25A | 993 |
| 395 | HALLMARK | G2M_CHECKPOINT | CDC25B | 994 |
| 396 | HALLMARK | G2M_CHECKPOINT | CDC27 | 996 |
| 397 | HALLMARK | G2M_CHECKPOINT | CDC45 | 8318 |
| 398 | HALLMARK | G2M_CHECKPOINT | CDC6 | 990 |
| 399 | HALLMARK | G2M_CHECKPOINT | CDC7 | 8317 |
| 400 | HALLMARK | G2M_CHECKPOINT | CDK1 | 983 |
| 401 | HALLMARK | G2M_CHECKPOINT | CDK4 | 1019 |
| 402 | HALLMARK | G2M_CHECKPOINT | CDKN1B | 1027 |
| 403 | HALLMARK | G2M_CHECKPOINT | CDKN2C | 1031 |
| 404 | HALLMARK | G2M_CHECKPOINT | CDKN3 | 1033 |
| 405 | HALLMARK | G2M_CHECKPOINT | CENPA | 1058 |
| 406 | HALLMARK | G2M_CHECKPOINT | CENPE | 1062 |
| 407 | HALLMARK | G2M_CHECKPOINT | CENPF | 1063 |
| 408 | HALLMARK | G2M_CHECKPOINT | CHAF1A | 10036 |
| 409 | HALLMARK | G2M_CHECKPOINT | CHEK1 | 1111 |
| 410 | HALLMARK | G2M_CHECKPOINT | CHMP1A | 5119 |
| 411 | HALLMARK | G2M_CHECKPOINT | CKS1B | 1163 |
| 412 | HALLMARK | G2M_CHECKPOINT | CKS2 | 1164 |
| 413 | HALLMARK | G2M_CHECKPOINT | CTCF | 10664 |
| 414 | HALLMARK | G2M_CHECKPOINT | CUL1 | 8454 |
| 415 | HALLMARK | G2M_CHECKPOINT | CUL3 | 8452 |
| 416 | HALLMARK | G2M_CHECKPOINT | CUL4A | 8451 |
| 417 | HALLMARK | G2M_CHECKPOINT | CUL5 | 8065 |
| 418 | HALLMARK | G2M_CHECKPOINT | DBF4 | 10926 |
| 419 | HALLMARK | G2M_CHECKPOINT | DDX39A | 10212 |
| 420 | HALLMARK | G2M_CHECKPOINT | DKC1 | 1736 |
| 421 | HALLMARK | G2M_CHECKPOINT | DMD | 1756 |
| 422 | HALLMARK | G2M_CHECKPOINT | DR1 | 1810 |
| 423 | HALLMARK | G2M_CHECKPOINT | DTYMK | 1841 |
| 424 | HALLMARK | G2M_CHECKPOINT | E2F1 | 1869 |
| 425 | HALLMARK | G2M_CHECKPOINT | E2F2 | 1870 |
| 426 | HALLMARK | G2M_CHECKPOINT | E2F3 | 1871 |
| 427 | HALLMARK | G2M_CHECKPOINT | E2F4 | 1874 |
| 428 | HALLMARK | G2M_CHECKPOINT | EFNA5 | 1946 |
| 429 | HALLMARK | G2M_CHECKPOINT | EGF | 1950 |
| 430 | HALLMARK | G2M_CHECKPOINT | ESPL1 | 9700 |
| 431 | HALLMARK | G2M_CHECKPOINT | EWSR1 | 2130 |
| 432 | HALLMARK | G2M_CHECKPOINT | EXO1 | 9156 |
| 433 | HALLMARK | G2M_CHECKPOINT | EZH2 | 2146 |
| 434 | HALLMARK | G2M_CHECKPOINT | FANCC | 2176 |
| 435 | HALLMARK | G2M_CHECKPOINT | FBXO5 | 26271 |
| 436 | HALLMARK | G2M_CHECKPOINT | FOXN3 | 1112 |
| 437 | HALLMARK | G2M_CHECKPOINT | G3BP1 | 10146 |
| 438 | HALLMARK | G2M_CHECKPOINT | GINS2 | 51659 |
| 439 | HALLMARK | G2M_CHECKPOINT | GSPT1 | 2935 |
| 440 | HALLMARK | G2M_CHECKPOINT | H2AFV | 94239 |
| 441 | HALLMARK | G2M_CHECKPOINT | H2AFX | 3014 |
| 442 | HALLMARK | G2M_CHECKPOINT | H2AFZ | 3015 |
| 443 | HALLMARK | G2M_CHECKPOINT | HIF1A | 3091 |
| 444 | HALLMARK | G2M_CHECKPOINT | HIRA | 7290 |
| 445 | HALLMARK | G2M_CHECKPOINT | HIST1H2BK | 85236 |
| 446 | HALLMARK | G2M_CHECKPOINT | HMGA1 | 3159 |
| 447 | HALLMARK | G2M_CHECKPOINT | HMGB3 | 3149 |
| 448 | HALLMARK | G2M_CHECKPOINT | HMGN2 | 3151 |
| 449 | HALLMARK | G2M_CHECKPOINT | HMMR | 3161 |
| 450 | HALLMARK | G2M_CHECKPOINT | HN1 | 51155 |
| 451 | HALLMARK | G2M_CHECKPOINT | HNRNPD | 3184 |
| 452 | HALLMARK | G2M_CHECKPOINT | HNRNPU | 3192 |
| 453 | HALLMARK | G2M_CHECKPOINT | HOXC10 | 3226 |
| 454 | HALLMARK | G2M_CHECKPOINT | HSPA8 | 3312 |
| 455 | HALLMARK | G2M_CHECKPOINT | HUS1 | 3364 |
| 456 | HALLMARK | G2M_CHECKPOINT | ILF3 | 3609 |
| 457 | HALLMARK | G2M_CHECKPOINT | INCENP | 3619 |
| 458 | HALLMARK | G2M_CHECKPOINT | KATNA1 | 11104 |
| 459 | HALLMARK | G2M_CHECKPOINT | KIF11 | 3832 |
| 460 | HALLMARK | G2M_CHECKPOINT | KIF15 | 56992 |
| 461 | HALLMARK | G2M_CHECKPOINT | KIF20B | 9585 |
| 462 | HALLMARK | G2M_CHECKPOINT | KIF22 | 3835 |
| 463 | HALLMARK | G2M_CHECKPOINT | KIF23 | 9493 |
| 464 | HALLMARK | G2M_CHECKPOINT | KIF2C | 11004 |
| 465 | HALLMARK | G2M_CHECKPOINT | KIF4A | 24137 |
| 466 | HALLMARK | G2M_CHECKPOINT | KIF5B | 3799 |
| 467 | HALLMARK | G2M_CHECKPOINT | KMT5A | 387893 |
| 468 | HALLMARK | G2M_CHECKPOINT | KPNA2 | 3838 |
| 469 | HALLMARK | G2M_CHECKPOINT | KPNB1 | 3837 |
| 470 | HALLMARK | G2M_CHECKPOINT | LBR | 3930 |
| 471 | HALLMARK | G2M_CHECKPOINT | LIG3 | 3980 |
| 472 | HALLMARK | G2M_CHECKPOINT | LMNB1 | 4001 |
| 473 | HALLMARK | G2M_CHECKPOINT | MAD2L1 | 4085 |
| 474 | HALLMARK | G2M_CHECKPOINT | MAPK14 | 1432 |
| 475 | HALLMARK | G2M_CHECKPOINT | MARCKS | 4082 |
| 476 | HALLMARK | G2M_CHECKPOINT | MCM2 | 4171 |
| 477 | HALLMARK | G2M_CHECKPOINT | MCM3 | 4172 |
| 478 | HALLMARK | G2M_CHECKPOINT | MCM5 | 4174 |
| 479 | HALLMARK | G2M_CHECKPOINT | MCM6 | 4175 |
| 480 | HALLMARK | G2M_CHECKPOINT | MEIS1 | 4211 |
| 481 | HALLMARK | G2M_CHECKPOINT | MEIS2 | 4212 |
| 482 | HALLMARK | G2M_CHECKPOINT | MKI67 | 4288 |
| 483 | HALLMARK | G2M_CHECKPOINT | MNAT1 | 4331 |
| 484 | HALLMARK | G2M_CHECKPOINT | MT2A | 4502 |
| 485 | HALLMARK | G2M_CHECKPOINT | MTF2 | 22823 |
| 486 | HALLMARK | G2M_CHECKPOINT | MYBL2 | 4605 |
| 487 | HALLMARK | G2M_CHECKPOINT | MYC | 4609 |
| 488 | HALLMARK | G2M_CHECKPOINT | NASP | 4678 |
| 489 | HALLMARK | G2M_CHECKPOINT | NCL | 4691 |
| 490 | HALLMARK | G2M_CHECKPOINT | NDC80 | 10403 |
| 491 | HALLMARK | G2M_CHECKPOINT | NEK2 | 4751 |
| 492 | HALLMARK | G2M_CHECKPOINT | NOLC1 | 9221 |
| 493 | HALLMARK | G2M_CHECKPOINT | NOTCH2 | 4853 |
| 494 | HALLMARK | G2M_CHECKPOINT | NUMA1 | 4926 |
| 495 | HALLMARK | G2M_CHECKPOINT | NUP50 | 10762 |
| 496 | HALLMARK | G2M_CHECKPOINT | NUP98 | 4928 |
| 497 | HALLMARK | G2M_CHECKPOINT | NUSAP1 | 51203 |
| 498 | HALLMARK | G2M_CHECKPOINT | ODC1 | 4953 |
| 499 | HALLMARK | G2M_CHECKPOINT | ODF2 | 4957 |
| 500 | HALLMARK | G2M_CHECKPOINT | ORC5 | 5001 |
| 501 | HALLMARK | G2M_CHECKPOINT | ORC6 | 23594 |
| 502 | HALLMARK | G2M_CHECKPOINT | PAFAH1B1 | 5048 |
| 503 | HALLMARK | G2M_CHECKPOINT | PAPD7 | 11044 |
| 504 | HALLMARK | G2M_CHECKPOINT | PBK | 55872 |
| 505 | HALLMARK | G2M_CHECKPOINT | PDS5B | 23047 |
| 506 | HALLMARK | G2M_CHECKPOINT | PLK1 | 5347 |
| 507 | HALLMARK | G2M_CHECKPOINT | PLK4 | 10733 |
| 508 | HALLMARK | G2M_CHECKPOINT | PML | 5371 |
| 509 | HALLMARK | G2M_CHECKPOINT | POLA2 | 23649 |
| 510 | HALLMARK | G2M_CHECKPOINT | POLE | 5426 |
| 511 | HALLMARK | G2M_CHECKPOINT | POLQ | 10721 |
| 512 | HALLMARK | G2M_CHECKPOINT | PRC1 | 9055 |
| 513 | HALLMARK | G2M_CHECKPOINT | PRIM2 | 5558 |
| 514 | HALLMARK | G2M_CHECKPOINT | PRMT5 | 10419 |
| 515 | HALLMARK | G2M_CHECKPOINT | PRPF4B | 8899 |
| 516 | HALLMARK | G2M_CHECKPOINT | PTTG1 | 9232 |
| 517 | HALLMARK | G2M_CHECKPOINT | PTTG3P | 26255 |
| 518 | HALLMARK | G2M_CHECKPOINT | PURA | 5813 |
| 519 | HALLMARK | G2M_CHECKPOINT | RACGAP1 | 29127 |
| 520 | HALLMARK | G2M_CHECKPOINT | RAD21 | 5885 |
| 521 | HALLMARK | G2M_CHECKPOINT | RAD23B | 5887 |
| 522 | HALLMARK | G2M_CHECKPOINT | RAD54L | 8438 |
| 523 | HALLMARK | G2M_CHECKPOINT | RASAL2 | 9462 |
| 524 | HALLMARK | G2M_CHECKPOINT | RBL1 | 5933 |
| 525 | HALLMARK | G2M_CHECKPOINT | RBM14 | 10432 |
| 526 | HALLMARK | G2M_CHECKPOINT | RPA2 | 6118 |
| 527 | HALLMARK | G2M_CHECKPOINT | RPS6KA5 | 9252 |
| 528 | HALLMARK | G2M_CHECKPOINT | SAP30 | 8819 |
| 529 | HALLMARK | G2M_CHECKPOINT | SFPQ | 6421 |
| 530 | HALLMARK | G2M_CHECKPOINT | SLC12A2 | 6558 |
| 531 | HALLMARK | G2M_CHECKPOINT | SLC38A1 | 81539 |
| 532 | HALLMARK | G2M_CHECKPOINT | SLC7A1 | 6541 |
| 533 | HALLMARK | G2M_CHECKPOINT | SLC7A5 | 8140 |
| 534 | HALLMARK | G2M_CHECKPOINT | SMAD3 | 4088 |
| 535 | HALLMARK | G2M_CHECKPOINT | SMARCC1 | 6599 |
| 536 | HALLMARK | G2M_CHECKPOINT | SMC1A | 8243 |
| 537 | HALLMARK | G2M_CHECKPOINT | SMC2 | 10592 |
| 538 | HALLMARK | G2M_CHECKPOINT | SMC4 | 10051 |
| 539 | HALLMARK | G2M_CHECKPOINT | SNRPD1 | 6632 |
| 540 | HALLMARK | G2M_CHECKPOINT | SQLE | 6713 |
| 541 | HALLMARK | G2M_CHECKPOINT | SRSF1 | 6426 |
| 542 | HALLMARK | G2M_CHECKPOINT | SRSF10 | 10772 |
| 543 | HALLMARK | G2M_CHECKPOINT | SRSF2 | 6427 |
| 544 | HALLMARK | G2M_CHECKPOINT | SS18 | 6760 |
| 545 | HALLMARK | G2M_CHECKPOINT | STAG1 | 10274 |
| 546 | HALLMARK | G2M_CHECKPOINT | STIL | 6491 |
| 547 | HALLMARK | G2M_CHECKPOINT | STMN1 | 3925 |
| 548 | HALLMARK | G2M_CHECKPOINT | SUV39H1 | 6839 |
| 549 | HALLMARK | G2M_CHECKPOINT | SYNCRIP | 10492 |
| 550 | HALLMARK | G2M_CHECKPOINT | TACC3 | 10460 |
| 551 | HALLMARK | G2M_CHECKPOINT | TFDP1 | 7027 |
| 552 | HALLMARK | G2M_CHECKPOINT | TGFB1 | 7040 |
| 553 | HALLMARK | G2M_CHECKPOINT | TLE3 | 7090 |
| 554 | HALLMARK | G2M_CHECKPOINT | TMPO | 7112 |
| 555 | HALLMARK | G2M_CHECKPOINT | TNPO2 | 30000 |
| 556 | HALLMARK | G2M_CHECKPOINT | TOP1 | 7150 |
| 557 | HALLMARK | G2M_CHECKPOINT | TOP2A | 7153 |
| 558 | HALLMARK | G2M_CHECKPOINT | TPX2 | 22974 |
| 559 | HALLMARK | G2M_CHECKPOINT | TRA2B | 6434 |
| 560 | HALLMARK | G2M_CHECKPOINT | TRAIP | 10293 |
| 561 | HALLMARK | G2M_CHECKPOINT | TROAP | 10024 |
| 562 | HALLMARK | G2M_CHECKPOINT | TTK | 7272 |
| 563 | HALLMARK | G2M_CHECKPOINT | UBE2C | 11065 |
| 564 | HALLMARK | G2M_CHECKPOINT | UBE2S | 27338 |
| 565 | HALLMARK | G2M_CHECKPOINT | UCK2 | 7371 |
| 566 | HALLMARK | G2M_CHECKPOINT | UPF1 | 5976 |
| 567 | HALLMARK | G2M_CHECKPOINT | WHSC1 | 7468 |
| 568 | HALLMARK | G2M_CHECKPOINT | WRN | 7486 |
| 569 | HALLMARK | G2M_CHECKPOINT | XPO1 | 7514 |
| 570 | HALLMARK | G2M_CHECKPOINT | YTHDC1 | 91746 |
| 571 | HALLMARK | G2M_CHECKPOINT | ZAK | 51776 |

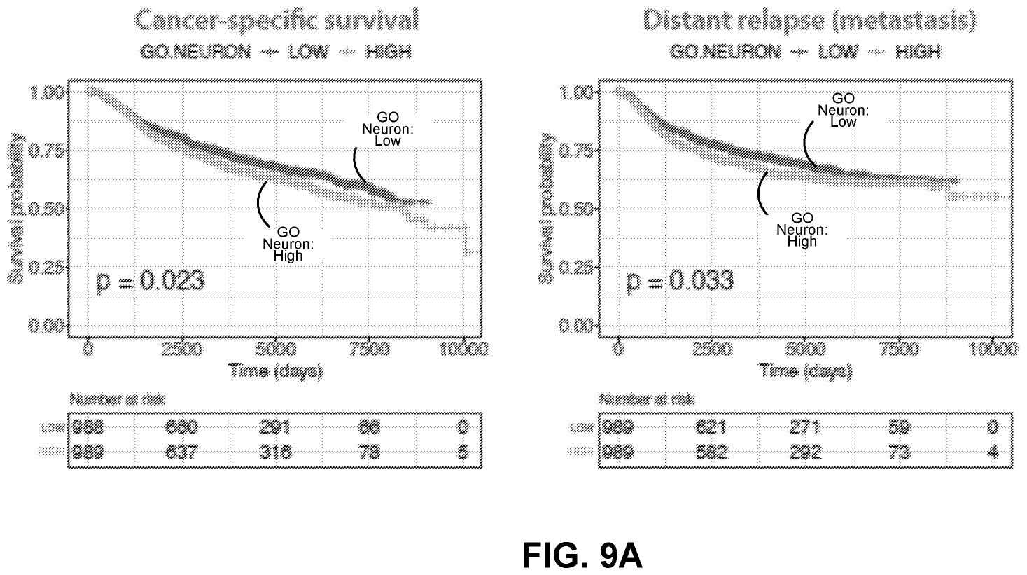

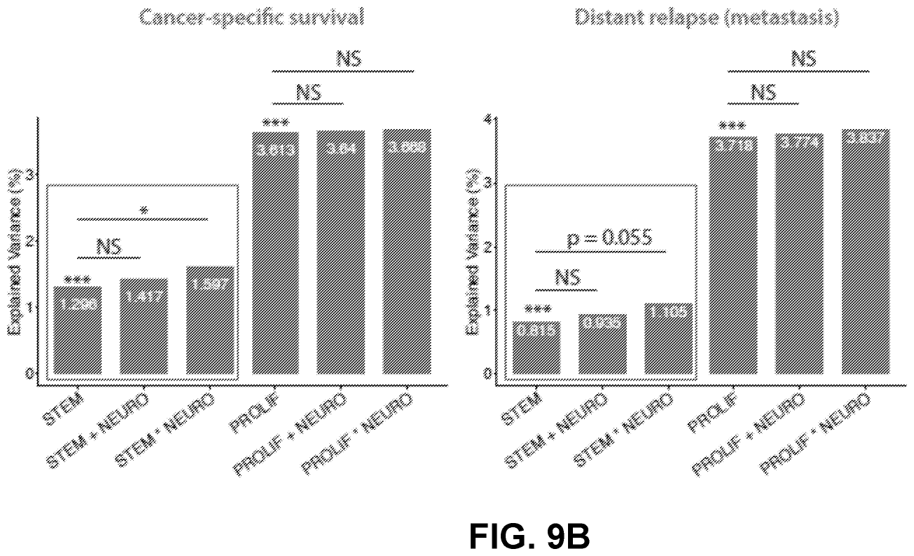

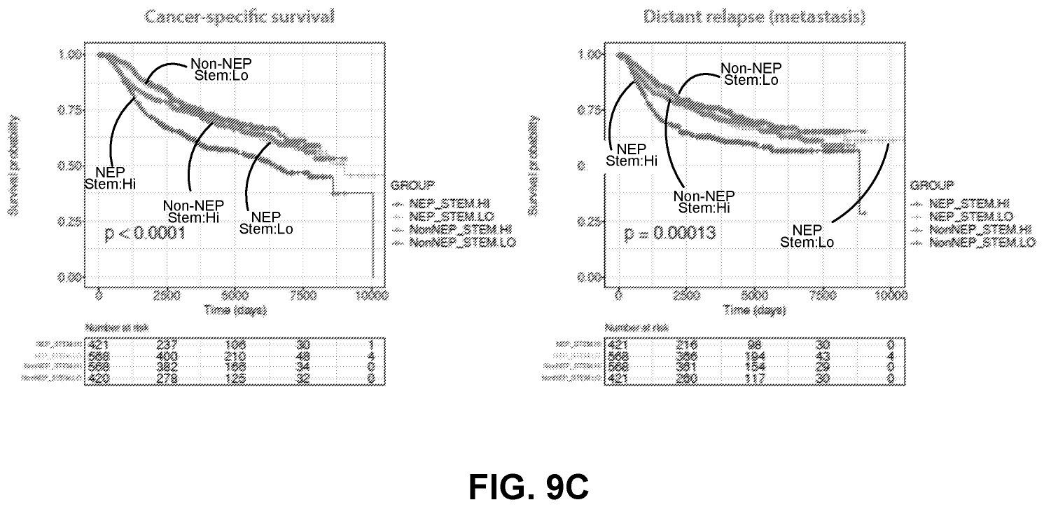

Survival modeling was then performed to determine an extent to which survival statistics differed across classifications. More specifically, data was retrieved from the largest publicly available breast cancer cohort METABRIC (N=1978) to investigate whether neural programming was associated with metastasis in humans. Here, the GO Neuron signature was used to score for neural programming, as RNA-Seq data were not available and classifying tumors as neurally related or not based on RNA-Seq data was thus not possible. Results indicated that neural programming was associated with decreased cancer-specific survival (CSS) and time to distant relapse (DR) (p=0.023 and 0.033 respectively, log-rank tests) (FIG. 9A). Next, to determine whether different types of neurally related tumors (well differentiated, low proliferating vs poorly differentiated high proliferating) were associated with different survival and metastasis associations, it was determined whether there was a significant statistical interaction between neural programming and either one of sternness or proliferation Among statistical models assessed, top performance was achieved when survival predictions were generated based on:

NEURO+STEMNESS+(NEURO*STEMNESS)

Even though both sternness and proliferation were significant prognostic factors for CSS and DR, only sternness had a significant interaction with neural programming (FIG. 9B). This suggested that poorly differentiated tumors may be more aggressive in the neurally programmed state. Visual assessment of Kaplan-Meier curves indicated that, indeed, poorly differentiated (stemness-high) NEP tumors were the most aggressive in terms of both CSS and DR (median cutoff for both sternness and GO Neuron scores). In contrast, well differentiated NEP tumors did not show a marked CSS or DR difference from non-NEP tumors (FIG. 9B). FIG. 9C shows Kaplan-Meier curves for the four cohorts (separated based on sternness and neurally relatedness). The cohort for the neurally related and high sternness classes were associated with the worst survival profile, but the other three groups were not statistically distinguishable. The results indicate that the neuralphenotype are associated with a risk factor to subjects beyond sternness alone.

### V.D. Example 4

Genetic expression data derived from human Small Cell Lung Cancer (SCLC) tumors (as described in George et al., “Comprehensive Genomic Profiles of Small Cell Lung Cancer” Nature. 2015 Aug. 6; 524(7563):47-53) were collected. Though SCLC tumors are generally known as a type of neuroendocrine indication (and thus being neurally related), a small subset of samples were, based on hierarchical clustering of samples (as implemented in accordance with the classification technique as described by George et al., “Comprehensive Genomic Profiles of Small Cell Lung Cancer” Nature. 2015 Aug. 6; 524(7563):47-53) as non-neuroendocrine. Thus, gene expression data for a first “NE” cohort (associated with the neuroendocrine characterization) was compared to gene expression data for a second “non-NE” cohort (associated with the non-neuroendocrine characterization). Immune cell signatures were adopted from CIBERSORT (Newman et al., “Robust enumeration of cell subsets from tissue expression profiles” Nat Methods. 2015 May; 12(5):453-7.) and included signatures for CD8 T cells, cytolytic activity and activated dendritic cells. The class I antigen presentation signature was adopted from Senbabaoglu et. al, “Tumor Immune Microenvironment Characterization in Clear Cell Renal Cell Carcinoma Identifies Prognostic and Immunotherapeutically Relevant Messenger RNA Signatures” Genome Biol. 2016 Nov. 17; 17(1):231. Signature scoring was performed by 1) computing z-scores for each gene across samples, and 2) computing the average of z-scores across genes in the signature. This process results in a score for each sample.

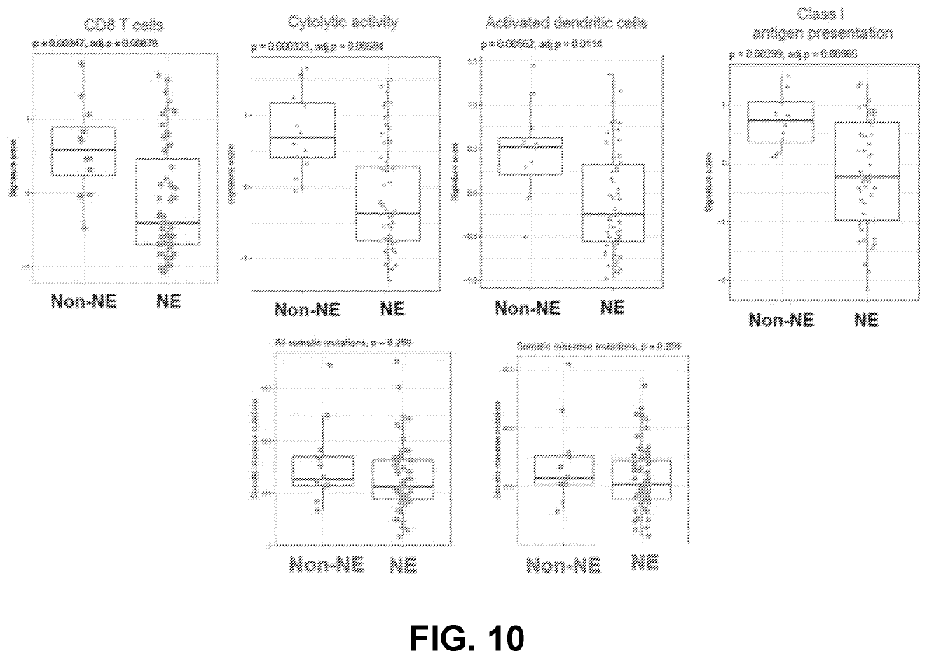

Scores for NE and non-NE groups are shown in the top-row plots of FIG. 10. Values for each of the four immune-cell signatures were higher for the non-neuroendocrine cohort as compared to the neuroendocrine cohort. These differences indicate that the neuroendocrine subtype of SCLC is low in immune infiltration compared to the non-neuroendocrine subtype in SCLC.

For each specimen, the number of somatic mutations and missense mutations was identified from data presented in from George et. al., 2015. Plots in the bottom row of FIG. 10 show the number of mutations for each cohort. The mutation counts of the neuroendocrine cohort are similar to those of the non-neuroendocrine cohort. This similarity indicates that the low immune infiltration in the neuroendocrine subtype cannot be explained by mutation load, because there is no significant difference between neuroendocrine and non-neuroendocrine cohorts in terms of mutation load.

### V.E. Example 5

V.E.1. Methods

V.E.1.a. Classifier Architecture

A gradient boosting machine (GrBM)-based classifier, termed NEPTUNE (Neurally Programmed Tumor PredictioN Engine) was trained using data sets downloaded from the Cancer Genome Atlas (TCGA) bulk RNA-Seq (available at https://gdc.cancer.gov/about-data/publications/pancanatlas) to predict whether a tumor was a neurally related tumor.

Selection of positive and negative cases: Known positive (i.e. neurally related) cases included samples from CNS indications such as glioblastoma (GBM, N=169) and low-grade glioma (LGG, N=534), as well as samples from the neuroendocrine indication pheochromocytoma and paraganglioma (PCPG, N=184). In addition, known positive cases also included samples from the TCGA pancreatic adenocarcinoma cohort that were subsequently removed from the study as they showed neuroendocrine histology (PAAD, N=8), samples from the TCGA lung adenocarcinoma cohort (LUAD) that were annotated as large cell neuroendocrine carcinoma (LCNEC, N=14), and samples from the TCGA muscle-invasive bladder cancer cohort that were discovered to form a gene expression-based “neuronal” subtype (as identified in accordance with the methodology of https://gdc.cancer.gov/about-data/publications/pancanatlas; BLCA, N=20). The total number of known positive samples added up to 929. FIG. 3 shows the distribution of tumor types included in each of the positive and negative sets.

Negative (i.e. non-neurally related) cases for all indications were included in the “positive” set that were not bona fide neuroendocrine or CNS indications. Thus, the “negative” set included samples from BLCA that were not annotated as neuroendocrine or not found to be in the gene expression-based “neuronal” subtype (N=387), samples from PAAD that were not annotated as neuroendocrine (N=171), samples from LUAD that were not annotated as LCNEC or not found to be in the gene expression-based “LCNEC-associated” subtype (N=427). The total number of negative cases was 985. (See FIG. 3.) The complement set was not used in the training set.

Preprocessing: Known positive and negative cases were altogether termed the “learning set” (N=1914). Preprocessing of the pan-cancer, batch effect-free TCGA RNA-Seq dataset included the following steps: 1) Subsetting to keep only the learning set tumor samples, 2) Log transformation with log 2(x+1) where x is RSEM values, and 3) Removing lowly expressed genes (high expression was defined as log-transformed RSEM-normalized expression levels being greater than 1 in at least 100 samples). These steps resulted in a data matrix of 18985 genes and 1914 samples.

Training and validation set split: The preprocessed data matrix was then randomly partitioned into training and validation sets with a 75%-25% split (FIG. 3). The distribution of positive and negative cases in each indication was maintained in the training and validation sets. Thus, the number of positive cases in the training and validation sets respectively were {127,42} for GBM, {401,133} for LGG, {138,46} for PCPG, {15,5} for BLCA, {11,3} for LUAD, and {6,2} for PAAD. The number of negative cases in the training and validation sets were {291,96} for BLCA, {321,106} for LUAD, and {129,42} for PAAD.

Feature selection with limma: Next, a differential expression test with limma was performed between positive and negative cases in the training set in order to identify the most discriminant and non-redundant genes for the classification task, as determined based on p-value ranks (FIG. 3). The validation set was not utilized for this step. In the limma linear model, each gene was regressed against a binary “neural phenotype” variable (positive or negative labels) as well as an indication factor to control for indication-specific expression patterns. The significance level for the differential expression of each gene was calculated using the treat method, which employs empirical Bayes moderated t-statistics with a minimum log-FC requirement. Of the 18,985, 1,969 genes (the discriminant set as discussed earlier with respect to FIG. 4) were associated with significant differences between positive and negative cases at adjusted p-value less than 0.1 and 1.5-fold difference (FIG. 3). The adjusted p-value and fold change thresholds were kept purposefully lenient as the goal of the analysis was to enrich for more discriminant genes for the training step. The NEPTUNE architecture contained 270 genes in total, those genes are listed in Table 1 above.

Training-set assessments: the NEPTUNE classifier was developed using the caret platform and gbm** package in R.

Performance of the NEPTUNE classifier was evaluated using the (‘centered and scaled’) training set. More specifically, the “centering and scaling” option in the caret function was used to subtract gene-specific average and divide by the standard deviation for the gene. Input was defined to be log transformed root-mean square error (RSEM) values. Hyperparameters were optimized using a grid search, and for each point in the grid, 5-fold cross-validation was performed with 10 repeats (50 total runs). The grid search was performed over two hyperparameters: 1) n.trees (number of trees in the ensemble) ranging from 50 to 500 with increments of 50, and 2) interaction.depth (complexity of the tree) selected from {1,3,5,7,9}. On the other hand, two other hyperparameters, namely shrinkage (the learning rate) and n.minobsinnode (the minimum number of training set samples in a node to commence splitting) were held constant at values of 0.1 and 10 respectively, as demonstrated in the caret package. An additional hyperparameter that was optimized for the classifier involved the choice of using the original ‘gene dimensions’ and also ‘principal components’ (PCA).

Because the problem being assessed was a two-class problem (NEP or non-NEP), the area under the ROC (AUROC) was selected as the performance metric. The AUROC for each point in the grid was an average of the AUROC values from the 50 resampling runs. For each resampling run, caret applied a series of cutoffs to the NEPTUNE score to predict the class. For each cutoff, sensitivity and specificity were computed for the predictions, and the ROC curve was generated across different cutoff values. The trapezoidal rule was used to compute AUROC.

NEPTUNE AUROC values in the training set were all higher than 0.995 across different values of hyperparameters (number of trees, depth of tree, ‘gene’ or ‘PCA’ dimensions). To evaluate performance on the validation set, hyperparameter values were selected to correspond to the highest AUROC (>0.995), and the number of miscalls in each indication was assessed.

Indication-specific performance was observed as being variable and relatively poorer in BLCA and LUAD (indications that are not bona fide neuroendocrine or nervous tissue tumors). The data thus suggested that a model optimized with cross-validation was robust to the choice of hyperparameters. In order to increase generalizability, it was decided to choose optimal hyperparameter values based on performance on the validation set. Interestingly, a random classifier, a gradient boosting architecture with 5 randomly selected genes (selected from the non-discriminant genes), also had high performance in the training set (AUROC values around 0.96). However, this performance partially broke down in the validation set with 44/475 false predictions (9.3%, gene dimensions), with potentially poorer performance in non-neuro/non-neuroendocrine indications (26/101 false predictions in BLCA, 25.7%, gene dimensions). The decrease in the performance of the random classifier in the validation set corroborated prior evidence that the cross-validated classifier may be prone to overfitting. However, this still relatively high performance of the random classifier suggested that the neurally related vs non-neurally related classification task can be performed relatively accurately even using comparatively small subsets of the discriminant set of genes. Biologically, this may stem from the fact that the brain tissue, together with blood, are the two major outgroups (thus easy to discriminate) in the human body from a gene expression perspective, and that many different sets of genes (even of relatively small size) may be informative in distinguishing them.

Validation-set assessments: To increase generalizability of the NEPTUNE classifier, hyperparameter values were optimized on the validation set. A grid search was applied for hyperparameter optimization with the same settings as those used in cross-validation (described above). However, F1-score was chosen as the performance metric in this step to be able to assess precision and recall simultaneously. F1-score was over 0.98 for the entire NEPTUNE grid, indicating that the general performance of the classifier was not sensitive to the choice of hyperparameters, again potentially pointing at the attainability of generating accurate classifications. A high value for tree depth was selected to allow for possible nonlinear interactions (interaction.depth=9) and a low value for number of trees was selected to reduce computational time (n.trees=50). The final classifier was then built by fitting a gradient boosted tree model to the learning set (training set+validation set) ‘gene dimensions’ using these hyperparameter values.

Computing platform: Training runs were parallelized into 5 copies of R using the doParallel** package, and executed in a high performance computing cluster.

Comparison of NEPTUNE to a logistic regression-based classifier: The NEPTUNE gradient boosting model was compared with a simpler architecture, L1-penalized logistic regression model, using the glmnet package, again within the R caret framework. Hyperparameter optimization in the logistic regression model was performed in a similar fashion to that for the gradient boosting model. A linear search was used to optimize the lambda hyperparameter. Possible values of lambda ranged from 0.001 to 0.1 by increments of 0.001, and the optimal value was determined to be 0.001 based on the F1-score from the validation set. Even though the logistic regression classifier had very similar performance as NEPTUNE, NEPTUNE had the advantage of being able to tolerate missing data. Tolerating missing data is advantageous for the extensibility of NEPTUNE to unseen datasets, because NEPTUNE was trained with Entrez Gene IDs from RefSeq, and datasets using other gene models are likely to have missing data due to the mismatch among gene models.

V.E.2. Results

V.E.2.a. A Machine Learning-Based Classifier Performs Better than alternative approaches in identifying NEP tumors.

High-throughput gene expression data can be used in multiple ways to call neurally related tumors in a pan-cancer cohort. These approaches include, in increasing level of sophistication, 1) individual neuronal/neuroendocrine marker genes, 2) neuronal/neuroendocrine signatures, 3) an unsupervised principal component analysis where new neurally related tumors would be called based on proximity to known neurally related tumors, and 4) a supervised machine learning approach where a classifier trained on known neurally related and non-neurally related tumors would predict new neurally related tumors.

Performance of these four approaches was tested in seven TCGA indications that had histopathology- or gene expression-based “neuronal” or “neuroendocrine” calls (both considered as neurally related in this instance). More specifically, performance of these four approaches was evaluated using a superset of data that included only high-confidence calls used in training. Histopathology-based neurally related tumors included central nervous system indications glioblastoma (GBM) and low-grade glioma (LGG), the neuroendocrine indication pheochromocytoma/paraganglioma (PCPG), 8 pancreatic neuroendocrine tumors (Pan-NET) found in the TCGA pancreatic adenocarcinoma (PAAD) study, 4 cases from the muscle-invasive bladder cancer (BLCA) study that were found by pathology re-review to have small cell/neuroendocrine histology (PMID 28988769), as well as 14 cases from the lung adenocarcinoma study that were found to share histology features with large cell neuroendocrine cancers (LCNEC) (PMC5344748). Gene expression-based neurally related tumors included cases from the “neuronal” subtype discovered in the BLCA study (PMID 28988769), and the LCNEC-associated AD.1 subtype discovered in a joint analysis of TCGA lung adenocarcinoma (LUAD) and lung squamous cell carcinoma (LUSC) cohorts (PMID 28988769). The majority of gene expression-based neurally related tumors lacked small cell and neuroendocrine histology.

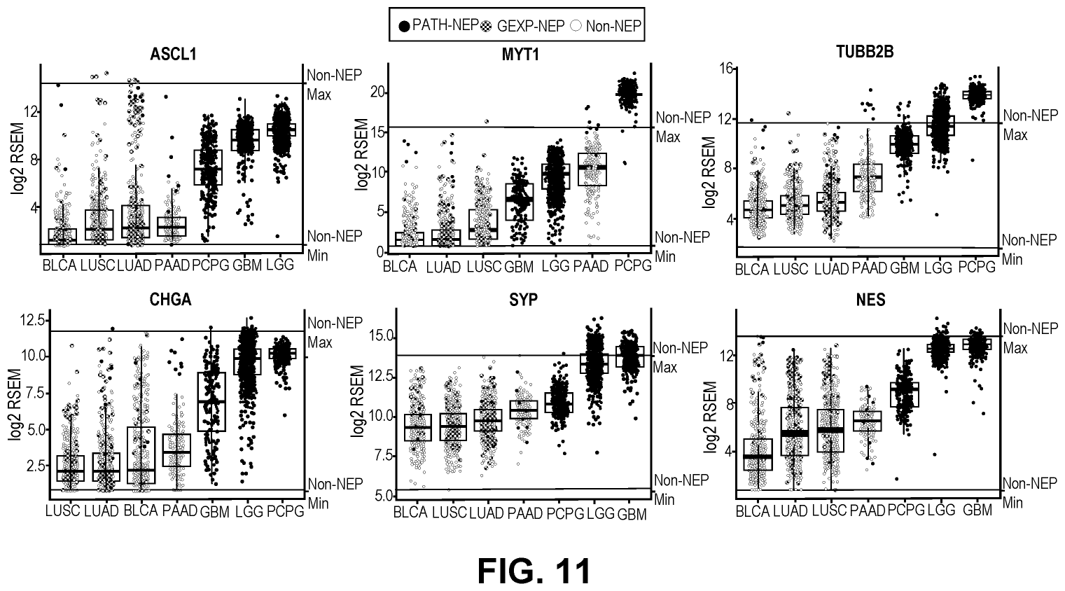

Six individual neuronal/neuroendocrine marker genes (ASCL 1, MYT1, CHGA, SYP, TUBB2B, NES) were selected and used to identify neurally related tumors in these seven indications. The maximum gene expression level in non-neurally related tumors did not successfully discriminate between neurally related (NEP) and non-neurally related (non-NEP) tumors. (FIG. 11.) Moreover, expression levels of an individual marker in neurally related and non-neurally related tumors overlap to a degree that prohibited the discovery of an effective cutoff in this approach. Further, gene expression-based neurally related calls, as opposed to the histopathology-based ones, were more difficult to distinguish from non-neurally related tumors using an individual marker approach, potentially owing to the fact that their initial discovery also depended on multi-dimensional clustering methods.

For the second approach, published neuroendocrine tumor (NET) (see: Tsai et al., “Gene Expression Signatures of Neuroendocrine Prostate Cancer and Primary Small Cell Prostatic Carcinoma” BMC Cancer. 2017 Nov. 13; 17(1):759, corresponding to rows 41-77 of Table 5; and Xu et al., “Pan-cancer transcriptome analysis reveals a gene expression signature for the identification of tumor tissue origin” Mod Pathol. 2016 June; 29(6):546-56, corresponding to rows 78-86 of Table 5), and neuronal (The Gene Ontology Consortium, 2019; Jassal et al., “The Reactome Pathway Knowledgebase” Nucleic Acids Res. 2020 Jan. 8; 48(D1):D498-D503; Robertson et. al., “Comprehensive Molecular Characterization of Muscle-Invasive Bladder Cancer” Cell. 2017 Oct. 19; 171(3):540-556, corresponding to rows 87-263 of Table 5) gene signatures, as well as a simpler 2-gene signature (SYP & NCAM1) that represented NET IHC markers were used to identify neurally related tumors.

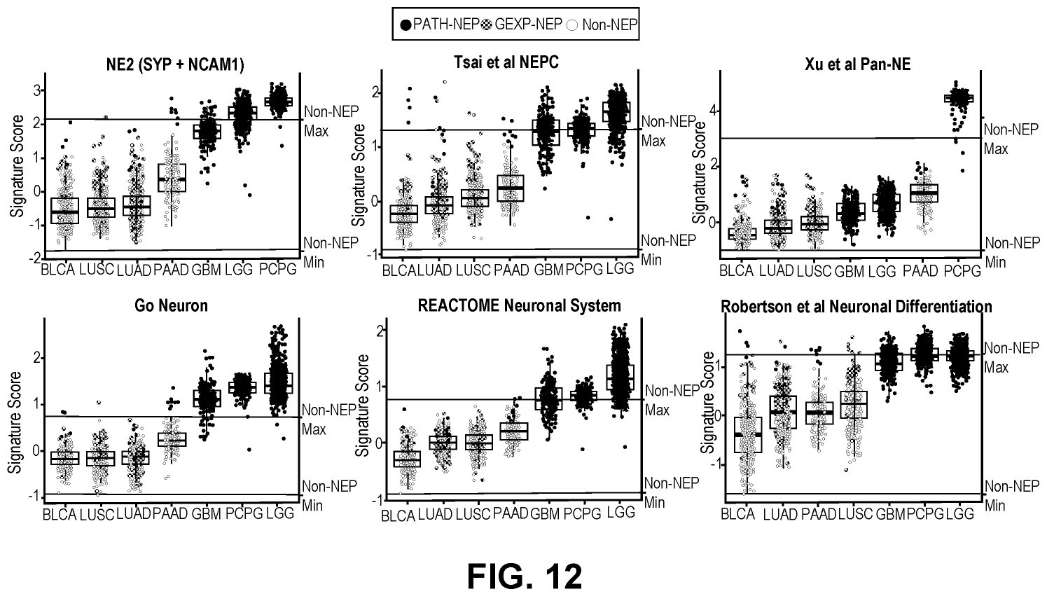

Performance metrics for the second approach exceeded those of the individual marker approach: The GO Neuron signature, in particular, was able to discriminate between neurally related and non-neurally related tumors to a better degree than other tested signatures and individual markers (FIG. 12). However, even this signature could not successfully capture LCNEC tumors in the LUAD cohort, or the large majority of gene expression-based neurally related tumors. Overall, none of the tested signatures or marker genes appeared specific enough for neurally related tumors. For a given signature and a given cancer indication, it could be possible to devise a cutoff that minimizes miscalls. However, FIGS. 11 and 12 indicate that the validity of any cutoff would be restricted to a small number of indications; it would not generalize to a pan-cancer setting.

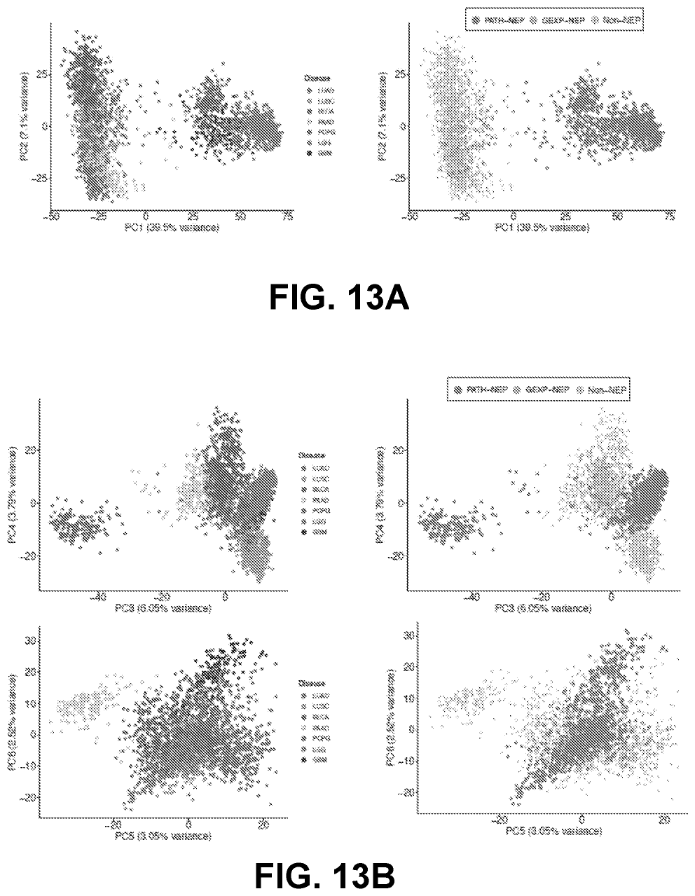

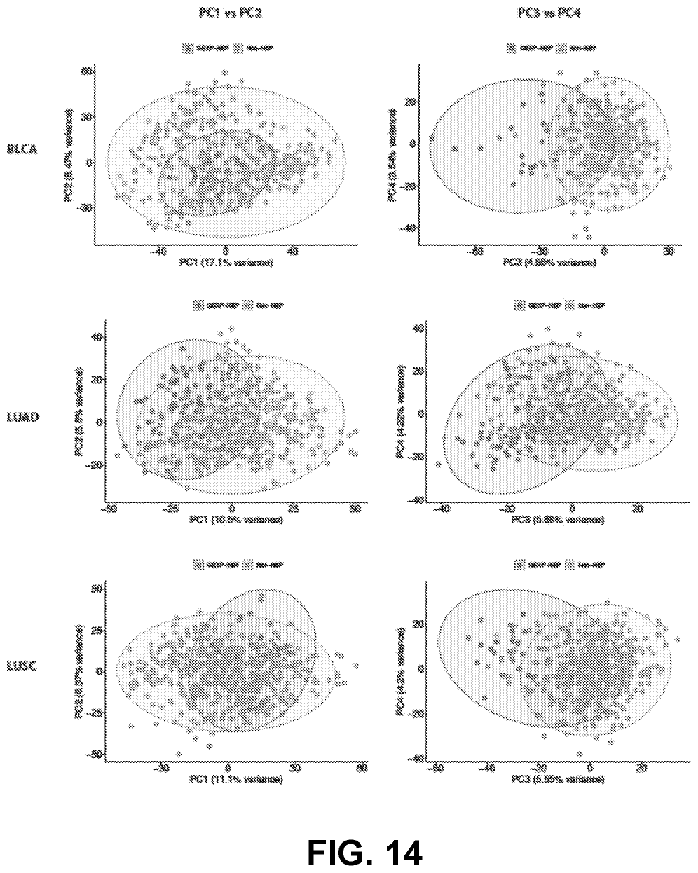

As a third approach, principal component analysis (PCA)—an unsupervised dimensionality reduction method—was used to identify clusters of neurally related tumors. A first principal component (PC1) was able to separate most histopathology-based neurally related tumors, with the exception of LCNEC tumors (FIG. 13A). Similar to the GO Neuron signature, PC1 (and also lower PCs) failed to identify LCNEC and gene expression-based neurally related tumors as separate neurally related clusters (FIGS. 13A-B). Thus, the data suggests that none of the individual-marker-gene; neuronal/neuroendocrine-signature; or PCA approach accurately predicted whether a tumor was neurally related based on gene-expression data.

Using the NEPTUNE supervised approach, the accuracy of positive (NEP) and negative (non-NEP) labels for training cases determines the performance of the resulting classifier in unseen datasets. Thus, only the high-confidence NEP calls from literature were included in the training set. As histopathology is an orthogonal piece of evidence with respect to gene expression, all histopathology-based NEP calls were considered as high-confidence. The gene expression-based NEP calls made in the BLCA, LUAD, and LUSC studies were scrutinized by assessing the separation between neurally related and non-neurally related tumors using principal component analysis. Gene expression-based NEP tumors were observed to form a distinct cluster—up to a low level of admixture—only in the BLCA study (FIG. 14). Therefore, gene expression-based NEP tumors from the BLCA study were included in the training set as high-confidence positive cases, but the ones from LUAD and LUSC were excluded. Consequently, the positive set included six indications (GBM, LGG, PCPG, LCNEC from LUAD, PanNET from PAAD, and BLCA-neuronal), and the negative set included non-neurally related samples from LUAD, PAAD and BLCA, with n-values as indicated in FIG. 3.

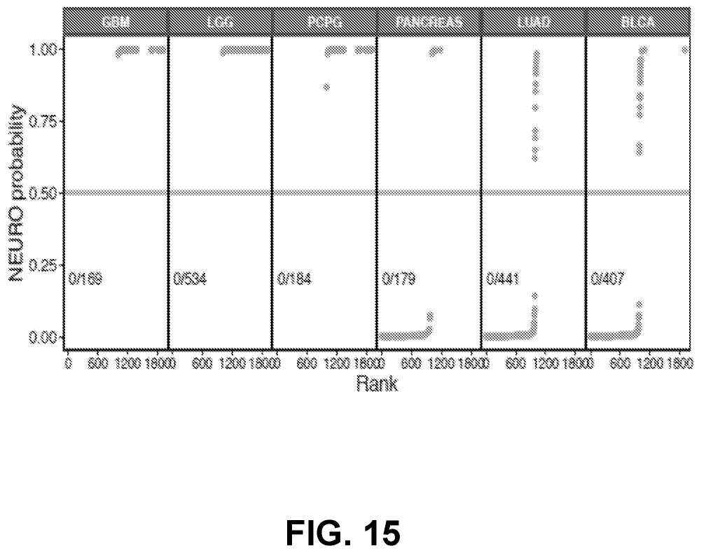

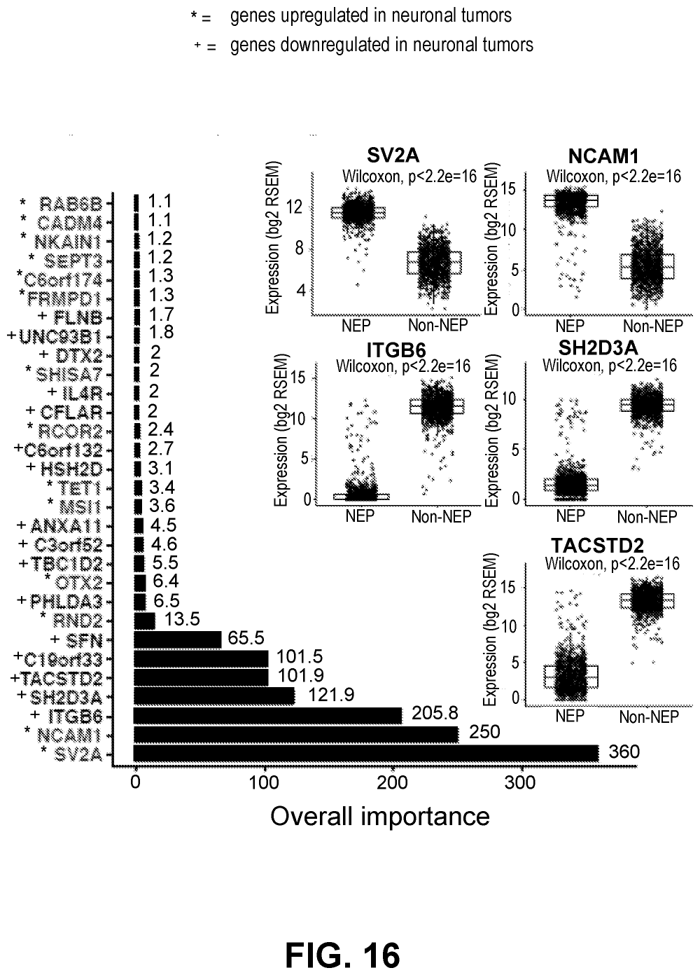

The NEPTUNE model was a highly accurate classifier with zero false positives and zero false negatives in the learning set (FIG. 15). As discussed above, NEPTUNE architecture contained 270 genes in total (Table 1), but only eight of these had importance score greater than 10 (FIG. 16). Genes upregulated or downregulated in NEP tumors were both found among top 8 classifier genes (FIG. 16 inset), with the upregulated genes indicating neuronal biology as expected (SV2A, NCAM1, RND2), and the downregulated genes suggesting loss of multiple functions including cell adhesion (ITGB6), cell cycle checkpoints and p53 activation (SFN[a]). Loss of cell cycle checkpoints may explain the proliferative phenotype, while proliferation alone previously was not predictive of efficacy of immune checkpoint blockade therapy.

V.E.2.b. NEPTUNE Finds More than Twice as Many Neurally Related Tumors as Those Already Known in TCGA

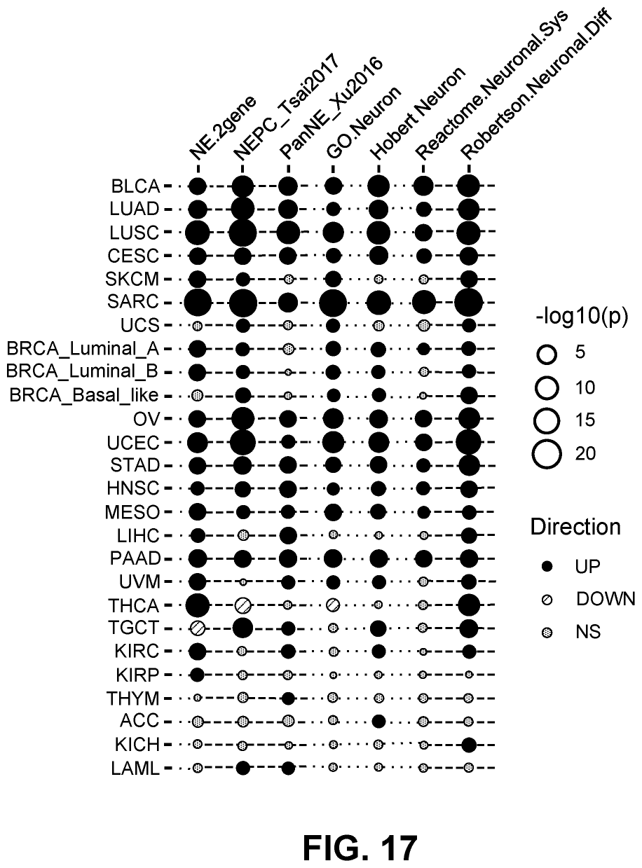

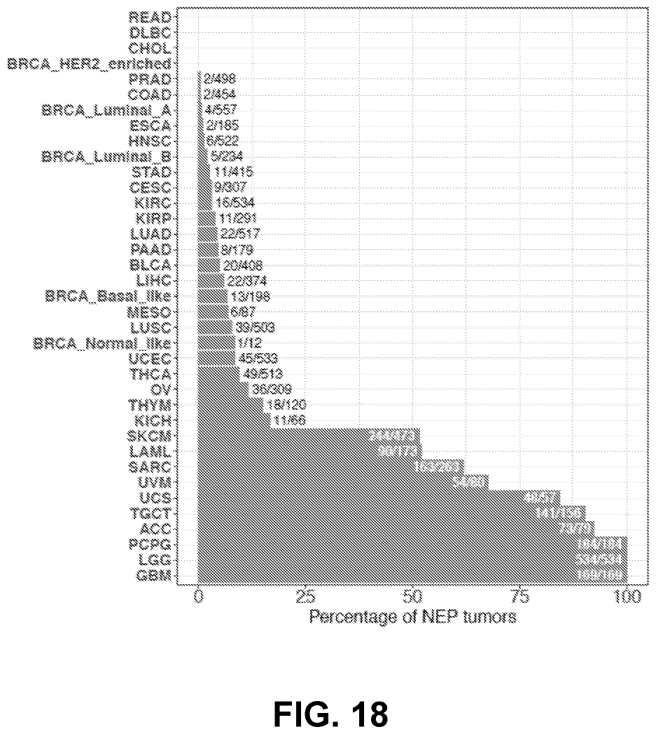

The NEPTUNE model was used to process gene-expression data from the TCGA holdout samples (not used for training or validation). Tumors predicted to be neurally related had elevated neuronal/neuroendocrine signature levels in all indications (FIG. 17). The NEPTUNE model predicted that 1129 that were not before known to be neurally related as having such classification. Along with the 929 positive cases in the learning set, the total number of tumor samples predicted to be neurally related was 2058 in TCGA (19.9% prevalence). The breakdown of 2058 NEP tumors by cancer indication showed that the prevalence of NEP tumors in untreated cohorts was greater than 50% in adrenocortical carcinoma (ACC), testicular germ cell tumors (TGCT), uterine carcinosarcoma (UCS), uveal melanoma (UVM), sarcoma (SARC), acute myeloid lymphoma (LAML), and skin cutaneous melanoma (SKCM) (FIG. 18).

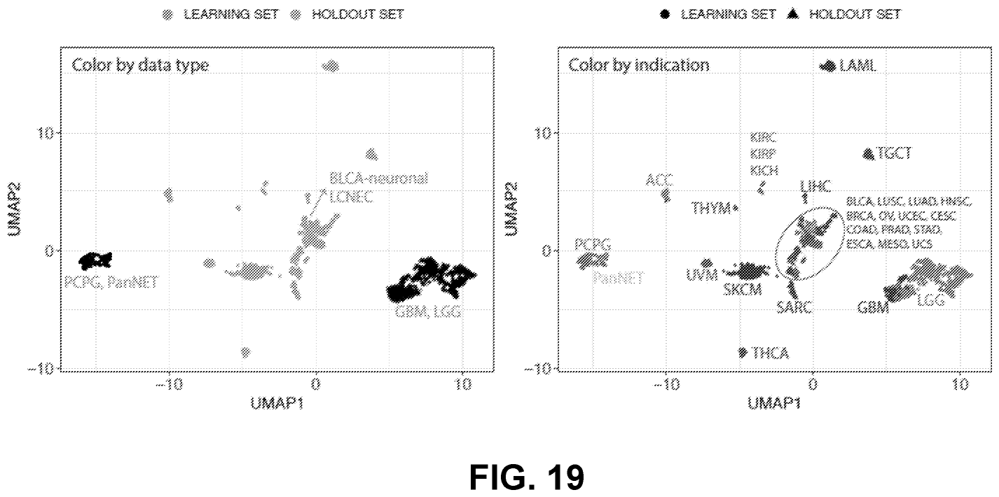

In training the NEPTUNE classifier, genes associated with individual indications were removed during the feature selection step in order to find genes that represented the pan-cancer neural programming biology. However, it was not certain whether the overrepresentation of GBM, LGG, and PCPG samples in the positive set still biased the classifier towards calling CNS-like or PCPG-like tumors. Thus, the instances from the holdout set predicted to be neurally related were compared with the instances from the learning set that were identified as being neurally related. In UMAP dimensions built from NEPTUNE's 270 genes, tumors from the holdout set that were predicted to be neurally related were more similar to positive training cases from BLCA and LCNEC (FIG. 19) than to CNS or neuroendocrine indications. Bona fide CNS and neuroendocrine indications (GBM, LGG, PCPG) formed separate clusters of their own. This data suggests that the NEPTUNE model was not biased towards individual CNS and neuroendocrine indications.

V.E.2.c. Neurally Related Tumors are Enriched in TCGA Subtypes in Multiple Indications

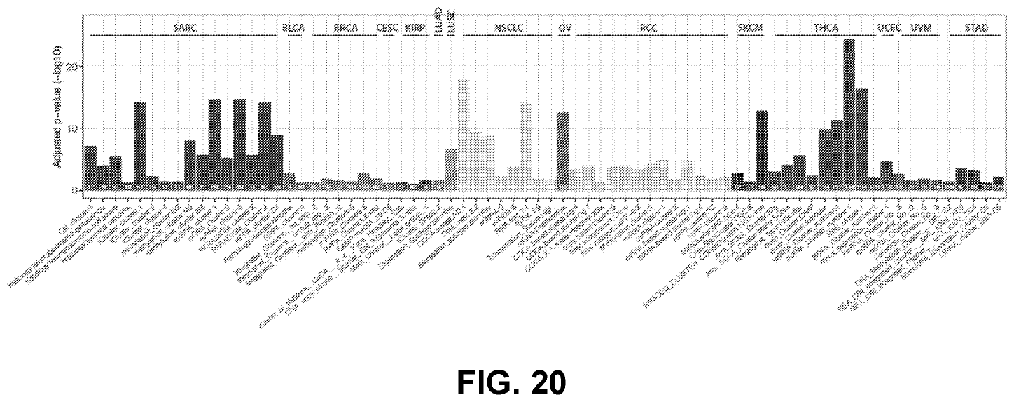

Potentially, enrichment of NEP tumors in TCGA subtypes provides information as to biological processes and pathways important for neural programming. Published TCGA subtype annotation from TCGAbiolinks, and an unbiased enrichment test (Fisher's exact test) was performed for tumors predicted to be neurally related. These tumors were significantly enriched in multiple subtypes including: 1) the “proliferative” subtype in ovarian cancer, 2) the smoking-associated “transversion high” subtype in NSCLC, 3) the “basal” subtype in breast cancer, 4) the “MITF-low” subtype in melanoma, 5) synovial sarcoma and leiomyosarcoma among all sarcoma, and 6) the “follicular”, “hypermethylator”, “CNV-rich”, and “22q loss” subtypes in papillary thyroid cancer (PTC) (FIG. 20).

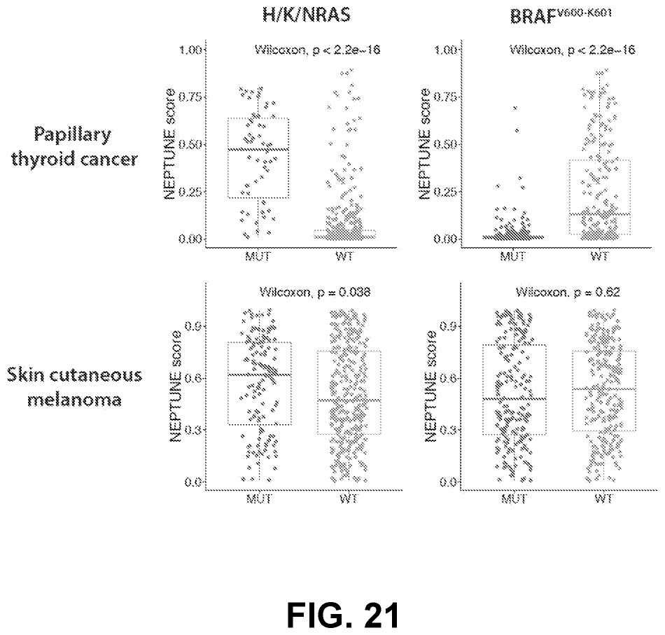

The mentioned PTC subtypes are largely from the more aggressive “RAS-like” subtype (and not the BRAFV600E-like subtype). Melanoma is another cancer indication with predominant RAS and BRAF mutant subtypes. (H/N/K)-RAS mutated samples had significantly higher NEPTUNE scores compared to RAS-wt samples in both PTC and melanoma (FIG. 21). The 22q loss subtype in PTC has no established driver, and in unbiased analysis, arm level 22q loss events were observed to be enriched in NEP tumors from not only PTC but also ovarian (OV), endometrial (UCEC) and lung squamous cell (LUSC) cancer. This finding suggests that 22q loss or neural programming may be driving the other in some tumors, or may have a common upstream driver.

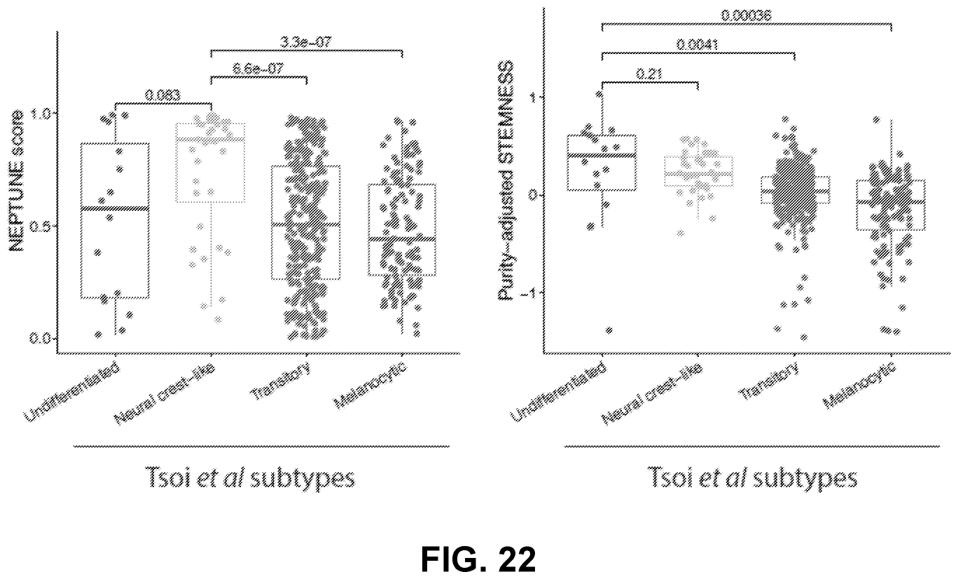

“MITF-low” is a poorly differentiated subtype in melanoma, as MITF is a differentiation factor in this indication. Given that NEP tumors were observed to be enriched in the MITF-low subtype, the “undifferentiated”, “neural crest-like”, “transitory”, and “melanocytic” subtype annotations were obtained from Tsoi et al. (“Multi-stage Differential Defines Melanoma Subtypes with Differential Vulnerability to Drug-Induced Iron-Dependent Oxidative Stress” Cancer Cell. 2018 May 14; 33(5):890-904). NEPTUNE scores were then compared across these subtypes. In melanoma, the NEPTUNE model called samples from the neural crest-like subtype with the highest scores, followed by those from the undifferentiated subtype (FIG. 22). These two subtypes also had the highest stemness scores, which suggested that NEPTUNE was successful in calling neurally related tumors, and neural related biology shared characteristics with stemness phenotype in some indications.

### VI. Example Use Cases

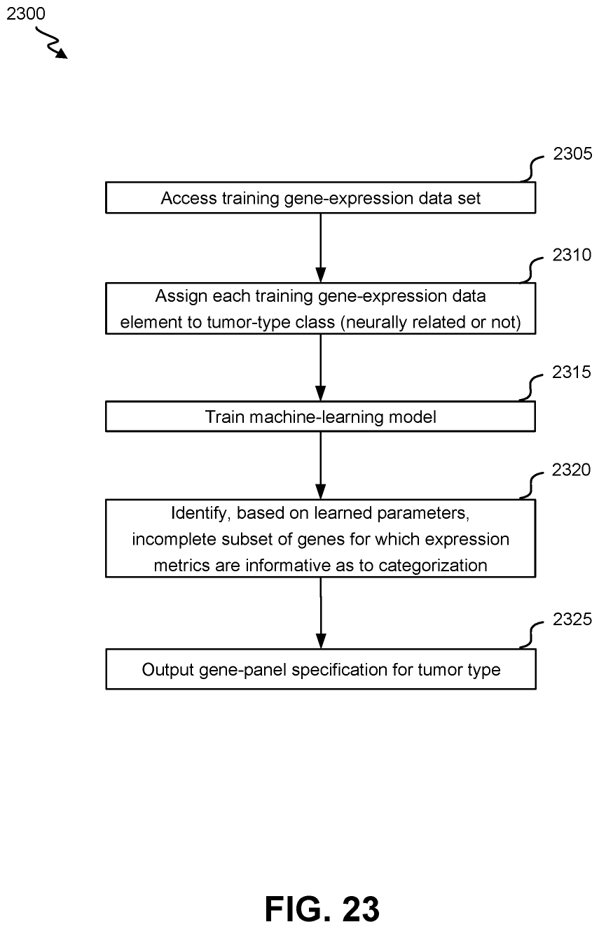

FIG. 23 illustrates a process 2300 of using a machine-learning model to identify a panel specification. At block 2305, a training gene-expression data set is accessed. The training gene-expression data set can include a set of data elements. Each data element can include, for each gene of a set of genes, expression data. Each data element can further include or be associated with a particular tumor type (e.g., associated with a body location or system) and/or a cell type).

At block 2310, each data element in the set of training gene-expression data set is assigned to a neurally related class or a non-neurally related class. The assignment may be based on rules. For example, a data element may be assigned to a neurally related class if associated tumor data indicates that a tumor is a brain tumor or neuroendocrine tumor (e.g., or any tumor that corresponds to a list item on a list of brain and/or neuroendocrine tumors) and to a non-neurally related class otherwise.

At block 2315, a machine-learning model is trained using the training data. The machine-learning model can be configured to receive gene-expression data and output a tumor class. Training the machine-learning model can include learning weights. In some instances, with respect to each gene, at least one weight represents a degree to which expression data for the gene is predictive of a tumor categorization. In some instances, there is no weight that solely corresponds to a single gene and/or any gene-specific weight is not representative of a degree to which expression data for the gene is predictive of a tumor categorization due to (for example) existence of other weights that pertain to the gene and other genes.

At block 2320, an incomplete subset of a set of genes is identified. Each gene of the subset may correspond to expression data for which it has been determined (based on learned parameter data and/or an output of the machine-learning model) is informative as to a tumor categorization assignment (e.g., neurally related or non-neurally related). In some instances, a weight is identified for each of a set of genes, and the incomplete subset can includes (and/or can be defined to be) those genes for which the weight exceeds an absolute or relative threshold (e.g., so as to identify 20 genes associated with the highest weights). The weight may include a learned parameter of the machine-learning model (e.g., associated with a connection between nodes in a neural network, a weight in an eigenvector, etc.). In some instances, a weight is determined based on implementing an interpretation technique so as to discover, based on learned parameters, an extent to which a gene's expression is predictive of a label assignment.

At block 2325, a gene-panel specification is output for the tumor type based on the identified incomplete subset, including an identity of some or all of the identified incomplete subset. The gene-panel specification may include an identity of each of the subset of genes to include in the panel. The gene-panel specification may be locally presented or transmitted to another computer system. Thus, the gene-panel specification can be used to design a gene panel useful for discriminating neurally related and non-neurally related tumors with respect to a given type of tumor (e.g., the type of tumor corresponding to a particular organ, anatomical location, cell type, etc.).

Thus, process 2300 can generated an output that can be used to facilitate a design of a gene panel that can be used to determine whether a tumor of a given subject is neurally related or non-neurally related. A gene panel may be designed accordingly, such that an expression level for each of the subset of genes is determined. The expression levels may then be assessed using the same machine-learning model, a different machine-learning model and/or a different technique to determine whether a tumor is neurally related.

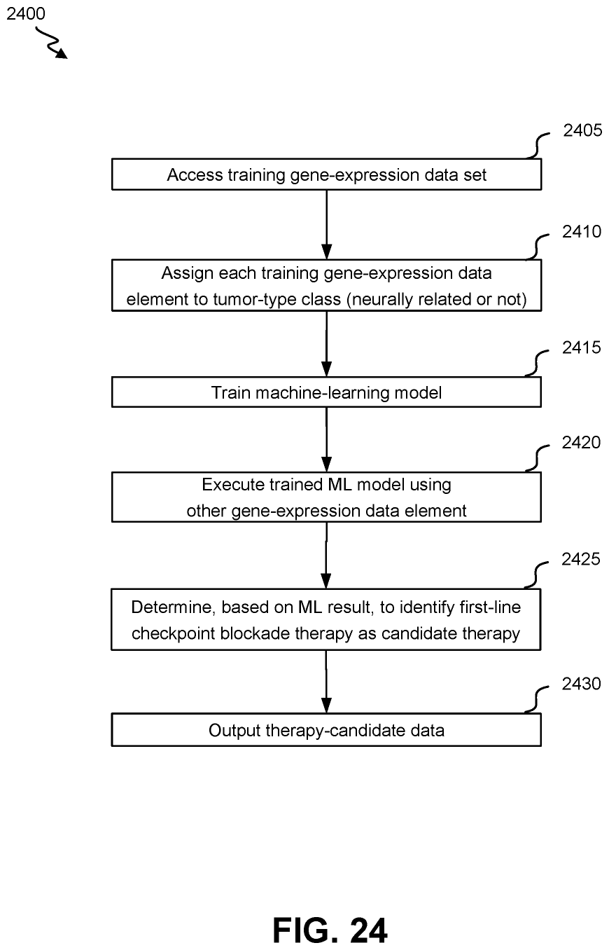

FIG. 24 illustrates a process 2400 of using a machine-learning model to identify therapy-candidate data. Blocks 2405-2415 of process 2400 parallel blocks 2305-2315 of process 2300. However, in some (but not all) instances, a configuration of the machine-learning model may be focused on a smaller set of genes as compared to the machine-learning model trained in block 2415. For example, the smaller set of genes may correspond to genes known to be in a given gene panel, genes identified as being within an incomplete subset (with the incomplete subset including genes that are informative as to a tumor's class), etc. For example, a machine-learning model may be initially trained based on expression data pertaining to a set of genes, a subset of the set of genes may be identified as being informative as to a tumor class, and the same machine-learning model or another machine-learning model can then be (re)trained based on the subset of the set of genes. For example, blocks 805-820 of process 800 may first be performed with training data that pertains to a set of genes, and blocks 2405-2415 or process 2400 may subsequently be performed with training data that pertains to a subset of the set of genes.

At block 2420, the trained machine-learning model is executed using another gene-expression data element. The other gene-expression data element can include expression data that corresponds to all or some of the genes represented in the training gene-expression data set accessed at block 2405. The other gene-expression data element may correspond to a particular subject who has a tumor. A result of the execution can include (for example) a probability that the tumor is of the neurally related class (or non-neurally related class), a confidence in the result and/or a categorical class assignment (e.g., identifying a neurally related class assignment or non-neurally related class assignment).

At block 2425, a determination is made based on the machine-learning result to identify a first-line checkpoint blockade therapy as a treatment candidate. The checkpoint blockade therapy can include one that amplifies T cell effector function by interfering with inhibitory pathways that would normally constrain T cell reactivity. The first-line checkpoint blockade therapy may be provided alongside or in place of chemotherapy and/or radiation therapy.

In some instances, block 2425 includes determining that a result of the machine-learning model includes or corresponds to an assignment to the neurally related class, as the checkpoint blockade therapy may be selectively identified as a first-line therapy in cases where a neurally related class assignment was generated. In some instances, a post-processing of the machine-learning result(s) may be performed to assess and/or transform the result(s) to a class assignment. For example, an assignment to the neurally related class may be made if a result indicates that a probability of such a class assignment exceeds 50% and an assignment to the non-neurally related class can be made otherwise.

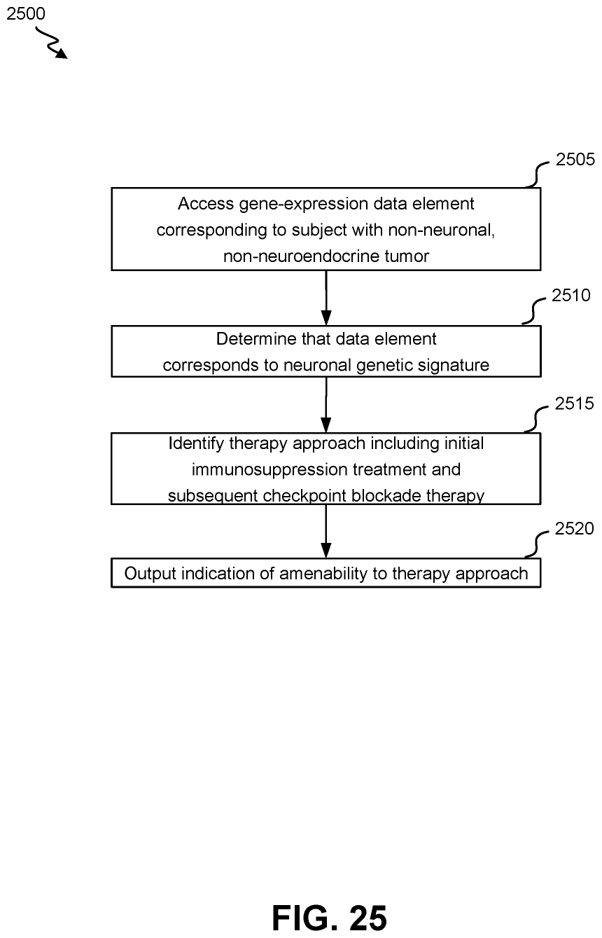

FIG. 25 illustrates a process 2500 of identifying a therapy amenability based on a neuronal-signature analysis. Process 2500 starts at block 2505 where a gene-expression data element is accessed. The gene-expression data element corresponds to a subject who has a tumor. The tumor can be a non-neuronal and non-neuroendocrine tumor. In some instances, the tumor is hot. The gene-expression data element can include expression data for each of a set of genes.

At block 2510, a determination is made that the data element corresponds to a neuronal genetic signature. The determination may include (for example) inputting part or all of the gene-expression data element (or a processed version thereof) to a machine-learning model. The determination may include detecting that an output from a machine-learning model corresponds to a neurally related class. The determination may be based upon comparing each of one, more or all of the expression levels in the gene-expression data element to a threshold (e.g., which may, but need not, be differentially set for different genes). Learned parameters may indicate whether, with respect to a particular gene's expression level, exceeding the threshold is indicative of a tumor being neurally related or non-neurally related.

At block 2515, a therapy approach is identified that differs from a first-line checkpoint blockade therapy (e.g., that includes an initial immunosuppression treatment and subsequent checkpoint blockade therapy). At block 2520, an indication of amenability to the therapy approach is output (e.g., locally presented or transmitted to another device). In some instances, another therapy approach is also output. For example, another therapy approach could include chemotherapy or radiation without the subsequent checkpoint blockade therapy. In some instances, an output may indicate that a first-line checkpoint blockade therapy has not been identified as a candidate treatment.

Notably, the determination that the data elements corresponds to a neuronal genetic signature (at block 2510) can be performed based on assessment of previous data associated with neurally related or non-neurally related classes. Thus, it may depend upon a new type of tumor classification. However, the classification need not be made at a tumor type level. As explained above, tumors showing a neurally related phenotypes have been identified in tumor types that are not commonly identified as neuronal or neuroendocrine tumors. In other words, the classification between neurally related or non-neurally related classes does not match known classifications such as those based on tumor types. For example, for a given tumor type, a tumor of the tumor type may be associated with a neurally related class and/or neuronal genetic signature for some subjects but, for other subjects, a tumor of the tumor type may be associated with a non-neurally related class and/or may not be associated with the neuronal genetic signature. Further, tumors assigned to a neurally related class (versus a non-neurally related class) and/or determined to correspond to a neuronal genetic signature can include cold tumors and hot tumors, and/or tumors assigned to a non-neurally related class and/or determined not to correspond to a neuronal genetic signature can include cold tumors and hot tumors.

Additionally, process 2500 indicates that, with respect to a tumor that is neither a brain tumor nor a neuroendocrine tumor, the tumor is identified as corresponding to a neuronal genetic signature and that a therapy is then selected based on this signature. Thus, a therapy that may typically not be used for a given tumor type (e.g., the type corresponding to a location or system associated with the tumor) may be identified as an option due to the signature.

### VII. Exemplary Embodiments

A first exemplary embodiment includes a computer-implemented method for identifying a gene panel for assessing checkpoint-blockade-therapy amenability, including: accessing a set of training gene-expression data including one or more training gene-expression data elements each corresponding to a respective subject, where each training gene-expression data element includes an expression metric for each of a set of genes measured in a sample collected from the respective subject; assigning each of the set of training gene-expression data elements to a tumor-type class, where the assignment includes: assigning each of a first subset of the set of training gene-expression data elements to a first tumor class, where the first subset includes a training gene-expression data element for which the tumor was a neuronal tumor; and assigning each of a second subset of the set of training gene-expression data elements to a second tumor class, where, for each training gene-expression data element of the second subset, the tumor was a non-neuronal and non-neuroendocrine tumor; training a machine-learning model using the set of training gene-expression data elements and the tumor class assignments, where training the machine-learning model includes learning a set of parameters; identifying, based on the learned set of parameters, an incomplete subset of the set of genes, where expression metrics for genes in the incomplete subset are informative as to tumor class assignments; and outputting a specification for a gene panel for assessing checkpoint-blockade-therapy amenability, the specification identifying each gene represented in the incomplete subset.

A second exemplary embodiment includes the first exemplary embodiment, where each of at least one neuronal tumor represented in the first subset is a brain tumor.

A third exemplary embodiment includes the first or second exemplary embodiment, where the first subset does not include training gene-expression data elements for which the tumor was a non-neuronal and non-neuroendocrine tumor.

A fourth exemplary embodiment includes any of the previous exemplary embodiments, where the specification for the gene panel corresponds to a recommendation that each gene in the incomplete subset be included in the gene panel and that each gene in the set of genes but not in the incomplete subset not be included in the gene panel.

A fifth exemplary embodiment includes any of the previous exemplary embodiments, where the first subset includes an additional training gene-expression data element for which the tumor was a neuroendocrine tumor, the neuroendocrine tumor being a tumor that has developed from cells of the neuroendocrine or nervous system and/or that has been assigned a neuroendocrine subtype using histopathology or expression-based tests.

A sixth exemplary embodiment includes any of the previous exemplary embodiments, where for each training gene-expression data element of the second subset, the tumor was a non-neuronal and non-neuroendocrine tumor derived from a respective type of organ or tissue, and at least one training gene-expression data element in the first subset is a gene-expression data element for which the tumor was a neuroendocrine tumor derived from the same of respective type organ or tissue.

A seventh exemplary embodiment includes any of the previous exemplary embodiments, where training the machine-learning model includes, for each gene of the set of genes, identifying a first expression-metric statistic indicating a degree to which the gene is expressed in cells corresponding to the first tumor class and identifying a second expression-metric statistic indicating a degree to which the gene is expressed in cells corresponding to the second tumor class, and where, for each gene of the incomplete subset, a difference between the first expression-metric statistic and the second expression-metric statistic exceeds a predefined threshold.

In some embodiments, the difference between the first expression-metric statistic and the second expression-metric statistic is a fold change estimate between the expression of the gene in gene-expression data elements in the first tumor class and the expression of the gene in gene expression data elements in the second tumor class, or a value derived from said fold change estimate (such as e.g. by log transformation).

In some embodiments, the first expression-metric statistic and/or the second expression-metric statistic is an estimate of the abundance of one or more transcripts of the gene in a sample or collection of samples.

An eighth exemplary embodiment includes any of the previous exemplary embodiments, where training the machine-learning model includes learning a set of conditions for one or more splits in one or more decision trees, and where the incomplete subset is identified based on evaluation of the set of conditions.

A ninth exemplary embodiment includes any of the first through seventh exemplary embodiments, where training the machine-learning model includes learning a set of weights, and where the incomplete subset is identified based on the set of weights.

A tenth exemplary embodiment includes any of the first through seventh exemplary where the machine-learning model uses a classification technique, and where the learned parameters correspond to a definition of a hyperplane.

A eleventh exemplary embodiment includes any of the first through eighth exemplary where the machine-learning model includes a gradient boosting machine.

A twelfth exemplary embodiment includes any of the first through eleventh exemplary further including: receiving a first gene-expression data element identifying expression metrics for genes represented in results of the gene panel as determined for a first subject; determining, based on the first gene-expression data element, that a first tumor corresponds to the first tumor class; outputting a first output identifying a combination therapy as a therapy candidate for the first subject, the combination therapy including an initial chemotherapy and subsequent checkpoint blockade therapy; receiving second gene-expression data element identifying expression metrics for genes represented in results of the gene panel as determined for a second subject; determining, based on the second gene-expression data element, that a second tumor corresponds to the second tumor class, where each of the first tumor and the second tumor were identified as a non-neuronal and non-neuroendocrine tumor and as corresponding to a same type of organ; and outputting a second output identifying a first-line checkpoint blockade therapy as a therapy candidate for the second subject.

In some embodiments, the method includes identifying a set of candidate genes as genes of the set of genes for which a difference between the first expression-metric statistic and the second expression-metric statistic exceeds a predefined threshold and training the machine-learning model includes training the machine-learning model using the identified set of candidate genes.

In some embodiments, the set of candidate genes includes genes of the set of genes for which a difference between the first expression-metric statistic and the second expression-metric statistic exceeds a predefined threshold, and an estimate of the statistical significance of the difference satisfies a further criterion. For example, the estimate of the statistical significance may be a p-value or adjusted p-value, and the further criterion may be that the (adjusted) p-value is below a predefined threshold.

In some embodiments, training the machine-learning model includes learning a set of conditions for one or more splits in one or more decision trees, and where the incomplete subset is identified based on evaluation of the set of conditions.

In some embodiments, the machine-leaning model is a neural network, support vector machine, a decision tree or a decision tree ensemble, such as a gradient boosted machine.

A thirteenth exemplary embodiments includes a computer-implemented method for assessing checkpoint-blockade-therapy amenability of one or more subjects having a tumor, the method including: identifying a gene panel for assessment of checkpoint-blockade-therapy amenability using the method of any of the first through eleventh exemplary embodiments; receiving a gene expression data element including an expression metric for each of a set of genes measured in a sample collected from a subject having a tumor, where the set of genes includes the gene panel; determining, based on the gene expression data, whether the tumor belongs to the first tumor class or the second tumor class, where determining includes determining whether the expression metrics for the genes in the gene panel are closer to those of tumors in the first tumor class or tumors in the second tumor class; and identifying a combination therapy as a therapy candidate if the tumor was determined to belong to the first tumor class, and/or identifying a first-line checkpoint blockade therapy as a therapy candidate if the tumor was determined to belong to the second tumor class, the combination therapy including an initial chemotherapy and subsequent checkpoint blockade therapy.

A fourteenth exemplary embodiment includes the thirteenth exemplary embodiment and further includes outputting the identified candidate therapy.

A fifteenth exemplary embodiment includes the thirteenth or fourteenth exemplary embodiment and further includes repeating the receiving, determining and identifying with a second gene expression data element, where each of the first tumor and the second tumor were identified as a non-neuronal and non-neuroendocrine tumor, and where each of the first and the second tumor were identified as tumors in a same type of organ.

In embodiments, the type of organ is the lung, bladder or pancreas.

A sixteenth exemplary embodiment includes a computer-implemented method for identifying a therapy candidate for a subject having a tumor, the method including: accessing a machine-learning model that has been trained by performing a set of operations including: accessing a set of training gene-expression data including one or more training gene-expression data elements each corresponding to a respective subject, where each training gene-expression data element includes an expression metric for each of a set of genes measured in a sample collected from the respective subject; assigning each of the set of training gene-expression data elements to a tumor-type class, where the assignment includes: assigning each of a first subset of the set of training gene-expression data elements to a first tumor class, where the first subset includes a training gene-expression data element for which the tumor was a neuronal tumor; and assigning each of a second subset of the set of training gene-expression data elements to a second tumor class, where, for each training gene-expression data element of the second subset, the tumor was a non-neuronal and non-neuroendocrine tumor; and training a machine-learning model using the set of training gene-expression data elements and the tumor class assignments, where training the machine-learning model includes learning a set of parameters; accessing another gene-expression data element having been generated based on an a biopsy of a tumor associated with another subject, the other gene-expression data element including another expression metric for each gene of at least some of the set of genes measured in the other sample; using the trained machine-learning model and the other gene-expression data element to generate a result indicating that the other tumor is of the second tumor-class type; and in response to the result, outputting an output identifying a first-line checkpoint blockade therapy as a therapy candidate.

In some embodiments, training the machine-learning model includes learning a set of conditions for one or more splits in one or more decision trees, and where the incomplete subset is identified based on evaluation of the set of conditions.

In some embodiments, the machine-leaning model is a neural network, support vector machine, a decision tree or a decision tree ensemble, such as a gradient boosted machine.

A seventeenth exemplary embodiment includes the sixteenth exemplary embodiment, where each neuronal tumor represented in the first subset is a brain tumor.

An eighteenth exemplary embodiment includes the sixteenth or seventeenth exemplary embodiment, where the first subset does not include training gene-expression data elements for which the tumor was a non-neuronal and non-neuroendocrine tumor.

A nineteenth exemplary embodiment includes any of the sixteenth through eighteenth exemplary embodiment, where an incomplete subset of the set of genes are identified as being informative as to tumor class assignments based on the learned set of parameters, and where the at least some of the set of genes includes the incomplete subset of the set of genes and not other genes in the set of genes that are not in the incomplete subset.

A twentieth exemplary embodiment includes any of the sixteenth through nineteenth exemplary embodiments, where the first subset includes an additional training gene-expression data element for which the tumor was a neuroendocrine tumor, the neuroendocrine tumor being a tumor that has developed from cells of the neuroendocrine or nervous system and/or that has been assigned a neuroendocrine subtype using histopathology or expression-based tests.

A twenty-first exemplary embodiment includes any of the sixteenth through twentieth exemplary embodiments, where for each training gene-expression data element of the second subset, the tumor was a non-neuronal and non-neuroendocrine tumor derived from a respective type of organ or tissue, and at least one training gene-expression data element in the first subset is a gene-expression data element for which the tumor was a neuroendocrine tumor derived from the same of respective type organ or tissue.

A twenty-second exemplary embodiment includes any of the sixteenth through twenty first exemplary embodiments, where the machine-learning model includes a gradient boosting machine.

A twenty-third exemplary embodiment includes any of the sixteenth through twenty second exemplary embodiments, where the machine-learning model includes one or more decision trees.

A twenty-fourth exemplary embodiment includes any of the sixteenth through twenty-third exemplary embodiments, where the other tumor is a melanoma tumor.

A twenty-fifth exemplary embodiment includes any of the sixteenth through twenty-fourth exemplary embodiments, further including: accessing an additional gene-expression data element having been generated based on an additional biopsy of an additional tumor, the additional tumor being of associated with a same anatomical location as the other tumor, the additional tumor being associated with an additional subject who distinct from the other subject; using the trained machine-learning model and the additional gene-expression data element to generate an additional result indicating that the additional tumor is of the first tumor-class type; and identifying a therapy other than a first line checkpoint blockade therapy as a therapy candidate for the additional subject if the trained machine learning model classifies the tumor of the further subject in the first tumor class.

A twenty-sixth exemplary embodiment includes the twenty-fifth exemplary embodiment, where the other therapy includes a combination therapy that includes a first-line chemotherapy and a subsequent checkpoint blockade therapy.

A twenty-seventh exemplary embodiment includes the twenty-fourth or twenty-sixth exemplary embodiment, where the additional tumor is a non-neuronal and non-neuroendocrine tumor.

A twenty-eighth exemplary embodiment includes a computer-implemented method for identifying a candidate therapy for a subject having a tumor including: accessing a gene-expression data element including an expression metric for each of a set of genes measured in a sample collected from the subject; determining that the gene-expression data element corresponds to a neuronal genetic signature; identifying a therapy approach that includes an initial chemotherapy treatment and a subsequent checkpoint blockade therapy; and outputting an indication that the subject is amenable to the therapy approach.

A twenty-ninth exemplary embodiment includes any of the twenty-sixth through twenty eighth exemplary embodiments, where determining that the gene-expression data element corresponds to a neuronal genetic signature includes classifying the gene-expression data element between a first class including tumors having the neuronal signature and a second class including tumors not having the neuronal signature, where tumors in the first and second class have different expression of the at least one gene.

A thirtieth exemplary embodiment includes a computer-implemented method for identifying a candidate therapy for a subject having a tumor including: accessing a gene-expression data element including an expression metric for each of a set of genes measured in a sample collected from the subject; determining that the gene-expression data element does not correspond to a neuronal genetic signature; identifying a therapy approach that includes initial use of checkpoint blockade therapy; and outputting an indication that the subject is amenable to the therapy approach.

A thirty-first exemplary embodiment includes the thirtieth exemplary embodiment, where the therapy approach does not include use of chemotherapy.

A thirty-second exemplary embodiment includes the thirtieth or thirty-first exemplary embodiment, where determining that the gene-expression data element does correspond to a neuronal genetic signature includes classifying the gene-expression data element between a first class including tumors having the neuronal signature and a second class including tumors not having the neuronal signature, where tumors in the first and second class have different expression of the at least one gene.

A thirty-third exemplary embodiment includes any of the twenty-eighth through thirty-second exemplary embodiments, further including: determining the neuronal genetic signature by training a classification algorithm using a training data set that includes: a set of training gene-expression data elements, each training gene-expression data element of the set of training gene-expression data elements indicating, for each gene of at least the multiple genes, an expression metric corresponding to the gene; and labeling data that associates: a first subset of the set of training gene-expression data elements with a first label, the first label being indicative of a tumor having a neuronal property; and a second subset of the set of training gene-expression data elements with a second label, the second label being indicative of a tumor not having the neuronal property.

A thirty-fourth exemplary embodiment includes any of the twenty-eighth through thirty-third exemplary embodiments, where the set of genes includes at least one gene selected from: SV2A, NCAM1, ITGB6, SH2D3A, TACSTD2, C29orf33, SFN, RND2, PHLDA3, OTX2, TBC1D2, C3orf52, ANXA11, MSI1, TET1, HSH2D, C6orf132, RCOR2, CFLAR, IL4R, SHISA7, DTX2, UNC93B1, and FLNB.

A thirty-fifth exemplary embodiment includes any of the twenty-eighth through thirty-third exemplary embodiments, where the set of genes includes at least five genes selected from: SV2A, NCAM1, ITGB6, SH2D3A, TACSTD2, C29orf33, SFN, RND2, PHLDA3, OTX2, TBC1D2, C3orf52, ANXA11, MSI1, TET1, HSH2D, C6orf132, RCOR2, CFLAR, IL4R, SHISA7, DTX2, UNC93B1, and FLNB.

A thirty-sixth exemplary embodiment includes a kit for detecting gene expressions indicative of whether tumors are neurally related including a set of primers, where each primer of the set of primers binds specifically to a gene listed in Table 1, and where the set of primers includes at least 5 primers.

A thirty-seventh exemplary embodiment includes the thirty-sixth exemplary embodiment, where the set of primers are used to indicate whether tumors are neurally related based on outputs from a machine-learning model generated based on input data sets that include expression data corresponding to one or more genes.

A thirty-eighth exemplary embodiment includes the thirty-sixth exemplary embodiment, where the set of primers are used to indicated whether tumors are neurally related based on outputs from a machine-learning model trained to differentiate expression levels of multiple genes in cells of neurally related tumor types as compared to expression levels of the multiple genes in cells of non-neurally related tumor types.

A thirty-ninth exemplary embodiment includes any of the thirty-sixth through thirty-eighth exemplary embodiments, where the set of primers includes an upstream primer targeting a sequence that is upstream of a gene of the set of genes and one or more downstream primers that target other sequences that are downstream of the gene of the set of genes. An amplification may include the whole gene.

A fortieth exemplary embodiment includes any of the thirty-sixth through thirty-ninth exemplary embodiments, where the set of primers includes primers targeting at least 10 genes.

A forty-first exemplary embodiment includes any of the thirty-sixth through fortieth exemplary embodiments, where the set of primers includes primers targeting at least 20 genes.

A forty-second exemplary embodiment includes any of the thirty-sixth through forty first exemplary embodiments, where, for each of the set of primers, the gene to which the primer binds is associated, in Table 1, with a weight above 5.0.

A forty-third exemplary embodiment includes any of the thirty-sixth through forty first exemplary embodiments, where, for each of the set of primers, the gene to which the primer binds is associated, in Table 1, with a weight above 1.0.

A forty-fourth exemplary embodiment includes any of the thirty-sixth through forty first exemplary embodiments, where, for each of the set of primers, the gene to which the primer binds is associated, in Table 1, with a weight above 0.5.

A forty-fifth exemplary embodiment includes a system including a kit as defined in any of the thirty-sixth through forty-fourth exemplary embodiments, and a computer-readable medium including instructions that, when executed by at least one processor, cause the processor to implement the method of any of the first through twenty-fifth exemplary embodiments.

A forty-sixth exemplary embodiment includes a method for predicting whether an individual having one or more tumors is likely to benefit from a treatment including an agent that enhances activity of immune cells, the method including measuring an expression level of each of one or more genes listed in Table 2 in a tumor sample that has been previously obtained from the individual, and using the expression levels of the one or more genes to predict whether the individual is likely to benefit from the treatment including the agent that enhances activity of immune cells.

A forty-seventh exemplary embodiment includes the forty-sixth exemplary embodiment, where using the expression levels of the one or more genes to identify whether the individual is one who may benefit from the treatment including the agent that enhances activity of immune cells includes: classifying the tumor between a first class including tumors that are not expected to benefit from the treatment including the agent that enhances activity of immune cells and a second class including tumors that are expected to benefit from the treatment including the agent that enhances activity of immune cells, where tumors in the first class and second classes differ with regard to expression of the one or more genes.

A forty-eighth exemplary embodiment includes the forty-sixth or forty-seventh exemplary embodiment, where the one or more genes listed in Table 2 include 5 or more genes listed in Table 2.

A forty-ninth exemplary embodiment includes the forty-sixth or forty-seventh exemplary embodiment, where the one or more genes listed in Table 2 include 10 or more genes listed in Table 2.

A fiftieth exemplary embodiment includes the forty-sixth or forty-seventh exemplary embodiment, where the one or more genes listed in Table 2 include 1 or more genes listed in Table 3.

A fifty-first exemplary embodiment includes the forty-sixth or forty-seventh exemplary embodiment, where the one or more genes listed in Table 2 include 5 or more genes listed in Table 3.

A fifty-second exemplary embodiment includes the forty-sixth or forty-seventh exemplary embodiment where the one or more genes listed in Table 2 include 10 or more genes listed in Table 3.

A fifty-third exemplary embodiment includes the forty-sixth or forty-seventh exemplary embodiment where the one or more genes listed in Table 2 include 1 or more genes listed in Table 4.

A fifty-fourth exemplary embodiment includes the forty-sixth or forty-seventh exemplary embodiment where the one or more genes listed in Table 2 include 5 or more genes listed in Table 4.

A fifty-fifth exemplary embodiment includes the forty-sixth or forty-seventh exemplary embodiment where the one or more genes listed in Table 2 include 10 or more genes listed in Table 4.

A fifty-sixth exemplary embodiment includes any of the forty-sixth through fifty-fifth exemplary embodiments, where the treatment including the agent that enhances activity of immune cells includes an immune blockade therapy.

A fifty-seventh exemplary embodiment includes any of the forty-sixth through fifty-sixth exemplary embodiments, where a trained machine-learning model having processed the expression levels of the one or more genes provided a classification result characterizing the one or more tumors as being non-neurally related, and where the individual is predicted to be one likely to benefit from the treatment based on the classification result.

A fifty-eighth exemplary embodiment includes any of the forty-sixth through fifty-seventh exemplary embodiments, where identifying whether the individual is one who may benefit from the treatment including the agent that enhances activity of immune cells includes using a machine-learning model that has been trained to classify tumors between a first class including tumors that are neurally related and a second class including tumors that are non-neurally related, where tumors in the first class are not expected to be more effectively treated with the treatment including the agent that enhances activity of immune cells as compared to other tumors in the second class.

A fifty-ninth exemplary embodiment includes the fifty-eighth exemplary embodiment, where the machine learning model that has been trained using a method as described in any of the first through eleventh exemplary embodiments.

A sixtieth exemplary embodiment includes a method for selecting immune blockade therapy as a treatment for an individual having one or more tumors, the method including measuring an expression level of each of one or more genes listed in Table 2 in a tumor sample from the individual, and using the expression levels of the one or more genes to predict that the individual is likely to benefit from the treatment including the immune blockade therapy.

A sixty-first exemplary embodiment includes the sixtieth exemplary embodiment, where the one or more genes listed in Table 2 include 5 or more genes listed in Table 2.

A sixty-second exemplary embodiment includes the sixtieth exemplary embodiment, where the one or more genes listed in Table 2 include 10 or more genes listed in Table 2.

A sixty-third exemplary embodiment includes the sixtieth exemplary embodiment, where the one or more genes listed in Table 2 include 1 or more genes listed in Table 3.

A sixty-fourth exemplary embodiment includes the sixtieth exemplary embodiment, where the one or more genes listed in Table 2 include 5 or more genes listed in Table 3.

A sixty-fifth exemplary embodiment includes the sixtieth exemplary embodiment, where the one or more genes listed in Table 2 include 10 or more genes listed in Table 3.

A sixty-sixth exemplary embodiment includes the sixtieth exemplary embodiment, where the one or more genes listed in Table 2 include 1 or more genes listed in Table 4.

A sixty-seventh exemplary embodiment includes the sixtieth exemplary embodiment, where the one or more genes listed in Table 2 include 5 or more genes listed in Table 4.

A sixty-eighth exemplary embodiment includes the sixtieth exemplary embodiment, where the one or more genes listed in Table 2 include 10 or more genes listed in Table 4.

A sixty-ninth exemplary embodiment includes any of the sixtieth through sixty-eighth exemplary embodiments, where a trained machine-learning model having processed the expression levels of the one or more genes provided a classification result characterizing the one or more tumors as being non-neurally related, and where the individual is identified as one who may benefit from the treatment based on the classification result.

A seventieth exemplary embodiment includes a method of treating an individual having cancer, the method including: (a) measuring an expression level of each of one or more genes listed in Table 2 in a tumor sample that has been previously obtained from an individual; (b) using the expression levels of the one or more genes to classify the tumor as being non-neurally related; and (c) administering an effective amount of a checkpoint blockade therapy to the individual.

A seventy-first exemplary embodiment includes the seventieth exemplary embodiment, where the one or more genes listed in Table 2 include 5 or more genes listed in Table 2.

A seventy-second exemplary embodiment includes the seventieth exemplary embodiment, where the one or more genes listed in Table 2 include 10 or more genes listed in Table 2.

A seventy-third exemplary embodiment includes the seventieth exemplary embodiment, where the one or more genes listed in Table 2 include 1 or more genes listed in Table 3.

A seventy-fourth exemplary embodiment includes the seventieth exemplary embodiment, where the one or more genes listed in Table 2 include 5 or more genes listed in Table 3.

A seventy-fifth exemplary embodiment includes the seventieth exemplary embodiment, where the one or more genes listed in Table 2 include 10 or more genes listed in Table 3.

A seventy-sixth exemplary embodiment includes the seventieth exemplary embodiment, where the one or more genes listed in Table 2 include 1 or more genes listed in Table 4.

A seventy-seventh exemplary embodiment includes the seventieth exemplary embodiment, where the one or more genes listed in Table 2 include 5 or more genes listed in Table 4.

A seventy-eighth exemplary embodiment includes the seventieth exemplary embodiment, where the one or more genes listed in Table 2 include 10 or more genes listed in Table 4.

A seventy-ninth exemplary embodiment includes any of the seventieth through seventy-eighth exemplary embodiments, where the expression level of the one or more genes were determined to indicate that the one or more tumors of the individual are non-neurally related based on a result generated by a trained machine-learning model having processed the expression levels of the one or more genes.

An eightieth exemplary embodiment includes a checkpoint blockade therapy for use in a method of treatment of an individual having cancer, the method including: (a) measuring an expression level of each of one or more genes listed in Table 2 in a tumor sample that has been previously obtained from an individual; (b) using the expression levels of the one or more genes to classify the tumor as being non-neurally related; and (c) administering an effective amount of a checkpoint blockade therapy to the individual.

An eighty-first exemplary embodiment includes the eightieth exemplary embodiment, where the one or more genes listed in Table 2 include 5 or more genes listed in Table 2.

An eighty-second exemplary embodiment includes the eightieth exemplary embodiment, where the one or more genes listed in Table 2 include 10 or more genes listed in Table 2.

An eighty-third exemplary embodiment includes the eightieth exemplary embodiment, where the one or more genes listed in Table 2 include 1 or more genes listed in Table 3.

An eighty-fourth exemplary embodiment includes the eightieth exemplary embodiment, where the one or more genes listed in Table 2 include 5 or more genes listed in Table 3.

An eighty-fifth exemplary embodiment includes the eightieth exemplary embodiment, where the one or more genes listed in Table 2 include 10 or more genes listed in Table 3.

An eighty-sixth exemplary embodiment includes the eightieth exemplary embodiment, where the one or more genes listed in Table 2 include 1 or more genes listed in Table 4.

An eighty-seventh exemplary embodiment includes the eightieth exemplary embodiment, where the one or more genes listed in Table 2 include 5 or more genes listed in Table 4.

An eighty-eighth exemplary embodiment includes the eightieth exemplary embodiment, where the one or more genes listed in Table 2 include 10 or more genes listed in Table 4.

An eighty-ninth exemplary embodiment includes any of the eightieth through eighty eighth exemplary embodiments, where the expression level of the one or more genes were determined to indicate that the one or more tumors of the individual are non-neurally related based on a result generated by a trained machine-learning model having processed the expression levels of the one or more genes.

A ninetieth exemplary embodiment includes a method of treating an individual having cancer, the method including administering to the individual an effective amount of an agent that enhances activity of immune cells, where the level of one or more genes listed in Table 2 in a sample from the individual has been determined to correspond to a non-neurally related classification.

A ninety-first exemplary embodiment includes the ninetieth exemplary embodiment, where the one or more genes listed in Table 2 include 5 or more genes listed in Table 2.

A ninety-second exemplary embodiment includes the ninetieth exemplary embodiment, where the one or more genes listed in Table 2 include 10 or more genes listed in Table 2.

A ninety-third exemplary embodiment includes the ninetieth exemplary embodiment, where the one or more genes listed in Table 2 include 1 or more genes listed in Table 3.

A ninety-fourth exemplary embodiment includes the ninetieth exemplary embodiment, where the one or more genes listed in Table 2 include 5 or more genes listed in Table 3.

A ninety-fifth exemplary embodiment includes the ninetieth exemplary embodiment, where the one or more genes listed in Table 2 include 10 or more genes listed in Table 3.

A ninety-sixth exemplary embodiment includes the ninetieth exemplary embodiment, where the one or more genes listed in Table 2 include 1 or more genes listed in Table 4.

A ninety-seventh exemplary embodiment includes the ninetieth exemplary embodiment, where the one or more genes listed in Table 2 include 5 or more genes listed in Table 4.

A ninety-eighth exemplary embodiment includes the ninetieth exemplary embodiment, where the one or more genes listed in Table 2 include 10 or more genes listed in Table 4.

A ninety-ninth exemplary embodiment includes any of the ninetieth through ninety eighth embodiments, where the expression level of the one or more genes were determined to indicate that the one or more tumors of the individual are non-neurally related based on a result generated by a trained machine-learning model having processed the expression levels of the one or more genes.

A one-hundredth exemplary embodiment includes a system including one or more data processors; and a non-transitory computer readable storage medium containing instructions which, when executed on the one or more data processors, cause the one or more data processors to perform part or all of one or more methods disclosed herein.

A one-hundred and first exemplary embodiment includes a system including one or more data processors; and a non-transitory computer readable storage medium containing instructions which, when executed on the one or more data processors, cause the one or more data processors to perform part or all of any of the first through thirty-fifth, forty-sixth through seventy-ninth and ninetieth through ninety-ninth exemplary embodiments.

A one-hundred and second exemplary embodiment includes a computer-program product tangibly embodied in a non-transitory machine-readable storage medium, including instructions configured to cause one or more data processors to perform part or all of one or more methods disclosed herein.

A one-hundred and third exemplary embodiment includes a computer-program product tangibly embodied in a non-transitory machine-readable storage medium, including instructions configured to cause one or more data processors to perform part or all of any of the first through thirty-fifth, forty-sixth through seventy-ninth and ninetieth through ninety-ninth exemplary embodiments.

### VIII. Additional Considerations

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

The description herein provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the description herein to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

1.-29. (canceled)

30. A computer-implemented method for identifying a candidate therapy for a subject having a tumor comprising:
accessing a gene-expression data element comprising an expression metric for each of a set of genes measured in a sample collected from the subject;
determining that the gene-expression data element does not correspond to a neuronal genetic signature;
identifying a therapy approach that includes initial use of checkpoint blockade therapy; and
outputting an indication that the subject is amenable to the therapy approach.

31. The computer-implemented method of claim 30, wherein the therapy approach does not include use of chemotherapy.

32. The computer-implemented method of claim 30, wherein determining that the gene-expression data element does not correspond to the neuronal genetic signature comprises classifying the gene-expression data element between a first class comprising tumors having the neuronal genetic signature and a second class comprising tumors not having the neuronal genetic signature, wherein tumors in the first and the second class have different expression of at least one gene.

33. The computer-implemented method of claim 30, further comprising:
determining the neuronal genetic signature by training a classification algorithm using a training data set that includes:
a set of training gene-expression data elements, each training gene-expression data element of the set of training gene-expression data elements indicating, for each gene of the set of genes, an expression metric corresponding to the gene; and
labeling data that associates:
a first subset of the set of training gene-expression data elements with a first label, the first label being indicative of a tumor having a neuronal property; and
a second subset of the set of training gene-expression data elements with a second label, the second label being indicative of a tumor not having the neuronal property.

34. The computer-implemented method of claim 30, wherein the set of genes comprises at least one gene selected from: SV2A, NCAM1, ITGB6, SH2D3A, TACSTD2, C29orf33, SFN, RND2, PHLDA3, OTX2, TBC1D2, C3orf52, ANXA11, MSI1, TET1, HSH2D, C6orf132, RCOR2, CFLAR, IL4R, SHISA7, DTX2, UNC93B1, and FLNB.

35. The computer-implemented method of claim 30, wherein the set of genes comprises at least five genes selected from: SV2A, NCAM1, ITGB6, SH2D3A, TACSTD2, C29orf33, SFN, RND2, PHLDA3, OTX2, TBC1D2, C3orf52, ANXA11, MSI1, TET1, HSH2D, C6orf132, RCOR2, CFLAR, IL4R, SHISA7, DTX2, UNC93B1, and FLNB.

36.-103. (canceled)

104. The computer-implemented method of claim 30, wherein the neuronal genetic signature comprises a set of genes that have been identified as informative of assignment to one of a first class of tumors comprising one or more neuronal tumors and a second class of tumors comprising one or more tumors that are each non-neural and non-neuroendocrine.

105. The computer-implemented method of claim 104, wherein the first class of tumors comprises at least one neuroendocrine tumor, the neuroendocrine tumor being a tumor that has developed from cells of the neuroendocrine or nervous system and/or that has been assigned a neuroendocrine subtype using histopathology or expression-based tests.

106. The computer-implemented method of claim 33, wherein for each training gene-expression data element of the second subset, the tumor was a non-neuronal and non-neuroendocrine tumor derived from a respective type of organ or tissue, and at least one training gene-expression data element in the first subset is a gene-expression data element for which the tumor was a neuroendocrine tumor derived from the same of respective type organ or tissue.

107. The computer-implemented method of claim 33, wherein training the machine-learning model includes, for each gene of the set of genes, identifying a first expression-metric statistic indicating a degree to which the gene is expressed in cells corresponding to the first tumor class and identifying a second expression-metric statistic indicating a degree to which the gene is expressed in cells corresponding to the second tumor class, and wherein, for each gene of the incomplete subset, a difference between the first expression-metric statistic and the second expression-metric statistic exceeds a predefined threshold.

108. The method of claim 30, wherein determining that the gene-expression data element does not correspond to a neuronal genetic signature comprises:
measuring an expression level of each of one or more genes listed in Table 2 in a tumor sample that has been previously obtained from the individual; and
using the expression levels of the one or more genes to predict whether the individual is likely to benefit from the treatment comprising the agent that enhances activity of immune cells.

109. The method of claim 108, wherein the one or more genes listed in Table 2 include 5 or more genes listed in Table 2.

110. The method of claim 108, wherein the one or more genes listed in Table 2 include 10 or more genes listed in Table 2.

111. The method of claim 108, wherein the one or more genes listed in Table 2 include 1 or more genes listed in Table 3.

112. The method of claim 108, wherein the one or more genes listed in Table 2 include 5 or more genes listed in Table 3.

113. The method of claim 108, wherein the one or more genes listed in Table 2 include 10 or more genes listed in Table 3.

114. The method of claim 108, wherein the one or more genes listed in Table 2 include 1 or more genes listed in Table 4.

115. The method of claim 108, wherein the one or more genes listed in Table 2 include 5 or more genes listed in Table 4.

116. The method of claim 108, wherein the one or more genes listed in Table 2 include 10 or more genes listed in Table 4.

117. One or more computer-readable non-transitory storage media embodying software comprising instructions operable when executed to:
access a gene-expression data element comprising an expression metric for each of a set of genes measured in a sample collected from the subject;
determine that the gene-expression data element does not correspond to a neuronal genetic signature;
identify a therapy approach that includes initial use of checkpoint blockade therapy; and
output an indication that the subject is amenable to the therapy approach.

118. A system comprising one or more processors and a memory coupled to the processors comprising instructions executable by the processors, the processors being operable when executing the instructions to:
access a gene-expression data element comprising an expression metric for each of a set of genes measured in a sample collected from the subject;
determine that the gene-expression data element does not correspond to a neuronal genetic signature;
identify a therapy approach that includes initial use of checkpoint blockade therapy; and
output an indication that the subject is amenable to the therapy approach.
